(19) United States
(12) Patent Application Publication
(10) Pub. No.: US 2024/0117005 A1
(43) Pub. Date: Apr. 11, 2024

(54) NOVEL BICYCLIC PEPTIDES

(71) Applicant: THE COUNCIL OF THE QUEENSLAND INSTITUTE OF MEDICAL RESEARCH, Queensland (AU)

(72) Inventor: Sudha RAO, Queensland (AU)

(21) Appl. No.: 18/273,200

(22) PCT Filed: Jan. 19, 2022
(86) PCT No.: PCT/AU2022/050023

§ 371 Date: Jul. 19, 2023

(30) Foreign Application Priority Data
Jan. 19, 2021 (AU) 2021900114

(51) Int. Cl. C07K 14/705; A61K 9/127; A61K 9/51; A61K 45/06; A61K 47/12; A61P 35/00; C07K 17/02

(52) U.S. Cl. CPC C07K 14/70532; A61K 9/127; A61K 9/5123; A61K 45/06; A61K 47/12; A61P 35/00; C07K 17/02; A61K 38/00

(57) ABSTRACT

Disclosed are polypeptides which are covalently bound to molecular scaffolds such that two or more peptide loops are subtended between attachment points to the scaffold. More particularly, the invention provides peptide mimetics of PD-L1. Also disclosed are multimeric binding complexes of polypeptides which are covalently bound to molecular scaffolds such that two or more peptide loops are subtended between attachment points to the scaffold that are mimetics of PD-L1. Also disclosed are methods of using said peptides in treating a disease or disorder mediated by PD-L1 nuclear localisation.

### RELATED APPLICATIONS

This application claims priority to Australian Provisional Application No. 2021900114 entitled “Novel Bicyclic Peptides” filed on 19 Jan. 2021, the contents of which are incorporated herein by reference in their entirety.

### FIELD OF THE INVENTION

This invention relates generally to polypeptides which are covalently bound to molecular scaffolds such that two or more peptide loops are subtended between attachment points to the scaffold. In particular, the invention describes peptide mimetics of PD-L1. The invention also relates to multimeric binding complexes of polypeptides which are covalently bound to molecular scaffolds such that two or more peptide loops are subtended between attachment points to the scaffold that are mimetics of PD-L1. The invention also includes drug conjugates comprising said peptides and complexes, conjugated to one or more effector and/or functional groups, to pharmaceutical compositions comprising said peptide ligands, complexes and drug conjugates and to the use of said peptide ligands and drug conjugates in preventing, suppressing or treating a disease or disorder mediated by PD-L1 nuclear localisation.

### BACKGROUND OF THE INVENTION

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavor to which this specification relates.

Programmed cell death protein-1 (PD-1) plays an important role in regulation of the immune system through its ability to regulate T cell activation and reduce the immune response. PD-1 is expressed on activated T cells (including immunosuppressive CD4+ T cells (Treg) and exhausted CD8+ T cells), B cells, myeloid dendritic cells (MDCs), monocytes, thymocytes and natural killer (NK) cells (Gianchecchi et al. (2013) Autoimmun. Rev., 12: 1091-1100).

The PD-1 signaling pathway contributes to the maintenance of central and peripheral tolerance in normal individuals, thereby avoiding destruction of normal host tissue. In the thymus, the interaction of PD-1 and its ligands suppresses positive selection, thereby inhibiting the transformation of CD4− CD8− double negative cells to CD4+ CD8+ double positive T cells (Keir et al. (2005) J. Immunol., 175: 7329-7379). Inhibition of self-reactive and inflammatory effector T cells that escape negative selection to avoid collateral immune-mediated tissue damage is dependent on the PD-1 signaling pathway (Keir et al. (2006) J. Exp. Med., 203: 883-895).

PD-1 is bound by two ligands: programmed cell death ligand-1 (PD-L1; B7-H1; CD274) and programmed cell death ligand-2 (PD-L2; B7-DC; CD273). PD-L1 is expressed on various cell types, including T cells, B cells, dendritic cells, macrophages, epithelial cells and endothelial cells (Chen et al. (2012) Clin Cancer Res, 18(24): 6580-6587; Herzberg et al. (2016) The Oncologist, 21:1-8). PD-L1 expression is also upregulated in many types of tumour cells and other cells in the local tumour environment (Herzberg et al., supra). PD-L2 is predominantly expressed on antigen-presenting cells such as monocytes, macrophages and dendritic cells, but expression may also be induced on a wide variety of other immune cells and non-immune cells depending on microenvironmental stimuli (Herzberg et al., supra; Kinter et al. (2008) J. Immunol., 181: 6738-6746; Zhong et al. (2007) Eur. J. Immunol., 37: 2405-2410; Messal et al. (2011) Mol. Immunol., 48: 2214-2219; Lesterhuis et al. (2011) Mol. Immunol., 49: 1-3).

PD-1, PD-L1 and PD-L2 are overexpressed by malignant cells and other cells in the local tumour environment. PD-1 is highly expressed on a large proportion of tumour-infiltrating lymphocytes (TILS) from many different tumour types and suppresses local effector immune responses. TIL expression of PD-1 is associated with impaired effector function (cytokine production and cytotoxic efficacy against tumour cells) and/or poor outcome in numerous tumour types (Thompson et al. (2007) Clin Cancer Res, 13(6): 1757-1761; Shi et al. (2011) Int. J. Cancer, 128: 887-896). PD-L1 expression has been found to strongly correlate with poor outcome in many tumour types, including kidney, ovarian, bladder, breast, urothelial, gastric and pancreatic cancer (Keir et al. (2008) Annu. Rev. Immunol., 26: 677-704; Shi et al. (2011) Int. J. Cancer, 128: 887-896). PD-L2 has been shown to be upregulated in a subset of tumours and has also been linked to poor outcome.

Interestingly, nuclear PD-L1 expression has been shown to be associated with short survival duration and chemoresistance in several tumour types, including prostate, colorectal and breast cancer (Satelli, et al. (2016) Scientific Reports, 6: 28910; Ghebeh, et al. (2010) Breast Cancer Res., 12: R48).

Accordingly, members of the PD-1 signaling pathway are important therapeutic targets for the treatment of cancer and new therapeutic agents targeting this pathway, particularly the nuclear localization of the members of the PD-1 signaling pathway, are desired.

Cyclic peptides are able to bind with high affinity and specificity to protein targets and hence are an attractive molecule class for the development of therapeutics. In fact, several cyclic peptides are already successfully used in the clinic, as for example the antibacterial peptide vancomycin, the immunosuppressant drug cyclosporine or the anti-cancer drug octreotide (Driggers et al., (2008), Nat Rev Drug Discov 7 (7), 608-24). Good binding properties result from a relatively large interaction surface formed between the peptide and the target as well as the reduced conformational flexibility of the cyclic structures. Typically, macrocycles bind to surfaces of several hundred square angstrom, as for example the cyclic peptide CXCR4 antagonist CVX15 (400 Å²; Wu et al., (2007), Science 330, 1066-71), a cyclic peptide with the Arg-Gly-Asp motif binding to integrin α-Vβ3 (355 Å²) (Xiong et al., (2002), Science 296 (5565), 151-5) or the cyclic peptide inhibitor upain-1 binding to urokinase-type plasminogen activator (603 Å²; Zhao et al., (2007), J Struct Biol 160 (1), 1-10).

Due to their cyclic configuration, peptide macrocycles are less flexible than linear peptides, leading to a smaller loss of entropy upon binding to targets and resulting in a higher binding affinity. The reduced flexibility also leads to locking target-specific conformations, increasing binding specificity compared to linear peptides. This effect has been exemplified by a potent and selective inhibitor of matrix metalloproteinase 8 (MMP-8) which lost its selectivity over other MMPs when its ring was opened (Cherney et al., (1998), J Med Chem 41 (11), 1749-51). The favourable binding properties achieved through macrocyclization are even more pronounced in multicyclic peptides having more than one peptide ring as for example in vancomycin, nisin and actinomycin.

Different research teams have previously tethered polypeptides with cysteine residues to a synthetic molecular structure (Kemp and McNamara (1985), J. Org. Chem; Timmerman et al., (2005), ChemBioChem). Meloen and co-workers had used tris(bromomethyl)benzene and related molecules for rapid and quantitative cyclisation of multiple peptide loops onto synthetic scaffolds for structural mimicry of protein surfaces (Timmerman et al., supra). Methods for the generation of candidate drug compounds wherein said compounds are generated by linking cysteine containing polypeptides to a molecular scaffold as for example 1,1′,1″-(1,3,5-triazinane-1,3,5-triyl)triprop-2-en-1-one (TATA) (Heinis et al., (2014) Angewandte Chemie, International Edition 53(6) 1602-1606).

Phage display-based combinatorial approaches have been developed to generate and screen large libraries of bicyclic peptides to targets of interest (Heinis et al., (2009), Nat Chem Biol 5 (7), 502-7 and International Patent Publication No. WO09/098450). Briefly, combinatorial libraries of linear peptides containing three cysteine residues and two regions of six random amino acids (Cys-(Xaa)₆-Cys-(Xaa)₆-Cys) were displayed on phage and cyclised by covalently linking the cysteine side chains to a small molecule scaffold.

### SUMMARY OF THE INVENTION

The present invention is predicated in part on the discovery that bicyclic PD-L1 peptide mimetics comprising an amino acid sequence according to Formula I are particularly efficacious in inhibiting or reducing the nuclear localization of PD-L1. Accordingly, the inventors have conceived that PD-L1 bicyclic peptide mimetics comprising an amino acid sequence according to Formula I can be used to inhibit nuclear localization of PD-L1. It is understood that the PD-L1 bicyclic peptide mimetics inhibit nuclear localisation by preventing or inhibiting the complex of PD-L1 and importin α (IMPα).

In one aspect, the present invention provides a PD-L1 bicyclic peptide mimetic comprising a polypeptide that comprises at least three cysteine residues, separated by at least two loop sequences, and a molecular scaffold which forms covalent bonds with the cysteine residues of the polypeptide such that at least two polypeptide loops are formed on the molecular scaffold, wherein the PD-L1 bicyclic peptide mimetic comprises an amino acid sequence of:

(Formula I)
X₁C₁LX₂X₃IFC₂X₄LRKGX₅C₃X₆X₇X₈X₉KX₁₀

or a modified derivative, or pharmaceutically acceptable salt, thereof;
wherein:
C₁, C₂, and C₃ represent first, second and third cysteine residues, respectively;
X₁ is absent or alanine;
X₂ is selected from any small amino acid (optionally, threonine, glycine, serine, or alanine)
X₃ is selected from any amino acid;
X₄ is selected from any amino acid;
X₅ is selected from any amino acid;
X₆ is selected from any nonpolar/neutral amino acid (e.g., methionine, alanine, leucine, proline, glycine, isoleucine, phenylalanine, tryptophan, valine, and norleucine);
X₇ is selected from any nonpolar/neutral amino acid (e.g., methionine, alanine, proline, leucine, glycine, isoleucine, phenylalanine, tryptophan, valine, and norleucine);
X₈ is selected from any amino acid;
X₉ is selected from any nonpolar/neutral amino acid (e.g., valine, alanine, glycine, methionine, leucine, proline, isoleucine, phenylalanine, tryptophan, and norleucine); and
X₁₀ is selected from any amino acid.

Typically, X₂ is selected from threonine and alanine. In some preferred embodiments, X₂ is threonine.

Typically, X₃ is selected from phenylalanine and alanine. In some preferred embodiments, X₃ is phenylalanine.

Typically, X₄ is selected from arginine and alanine. In some preferred embodiments, X₄ is arginine.

Typically, X₅ is selected from arginine and alanine. In some preferred embodiments, X₅ is arginine.

Typically, X₆ is selected from methionine, alanine, leucine and proline. In some preferred embodiments, X₆ is methionine.

Typically, X₇ is selected from methionine, alanine, and proline. In some preferred embodiments, X₇ is methionine. In some alternative embodiments, X₇ is alanine.

Typically, X₈ is selected from aspartic acid, alanine, glycine and valine. In some preferred embodiments, X₈ is aspartic acid.

Typically, X₉ is selected from valine, alanine, glycine, methionine. In some preferred embodiments, X₉ is valine.

Typically, X₁₀ is selected from lysine, alanine, asparagine, and methionine. In some preferred embodiments, X₁₀ is lysine.

In some embodiments, the amino acid sequence of the PD-L1 bicyclic peptide mimetic comprises, consists, or consists essentially of: ACLTFIFCRLRKGRCMMDVKK [SEQ ID NO: 1].

In some embodiments, the PD-L1 bicyclic peptide mimetic binds to IMPα and prevents the complexation of IMPα and PD-L1. Typically, the PD-L1 bicyclic peptide mimetic does not inhibit or prevent the nuclear transport of other cellular proteins (e.g., PD-L2) by IMPα. Accordingly the PD-L1 bicyclic peptide mimetics specifically prevent the nuclear transport of PD-L1, but does not inhibit the nuclear transport of other proteins.

Suitably, the molecular scaffold comprises a (hetero)aromatic or (hetero)alicyclic moiety. Suitably, the scaffold comprises a tris-substituted (hetero)aromatic or (hetero)alicyclic moiety, for example a tris-methylene substituted (hetero)aromatic or (hetero)alicyclic moiety. The (hetero)aromatic or (hetero)alicyclic moiety is suitably a six-membered ring structure, preferably tris-substituted such that the scaffold has a 3-fold symmetry axis. In some preferred embodiments, the molecular scaffold is 1,3,5-(tribromoMethyl)benzene (TBMB). In other preferred embodiments, the molecular scaffold is a 1,3,5-tris-(bromoacetamido)benzene (TBAB).

In preferred embodiments, the bicyclic peptide further comprises an N-terminal cell-penetrating peptide. Preferably, the cell-penetrating peptide is Myr.

In some embodiments, the modified derivative includes one or more modifications selected from: N-terminal and/or C-terminal modifications; replacement of one or more amino acid residues with one or more non-natural amino acid residues (such a replacement of one or more polar amino acids with one or more isosteric or isoelectronic amino acids; replacement or one or more hydrophobic amino acid residues with other non-natural isosteric or isoelectronic amino acids); addition of a spacer group; replacement of one or more oxidation resistant amino acid residues; replacement of one or more amino acid residues with an alanine, replacement of one or more L amino acid residues with one or more D-amino acid residues; N-alkylation of one or more amide bonds within the bicyclic peptide ligand; replacement of one or more peptide bonds with a surrogate bond; peptide backbone length modification; substitution of the hydrogen on the α-carbon of one or more amino acid residues with another chemical group, and post-synthetic biorthogonal modification of amino acids such as cysteine, lysine, glutamate and tyrosine with suitable amine, thiol, carboxylic acid and phenol-reactive reagents.

In some embodiments, the modified derivative comprises an N-terminal modification, such as an N-terminal acetyl group.

In some embodiments, the modified derivative comprises a C-terminal modification, such as a C-terminal amide group.

In some embodiments, the modified derivative comprises replacement of one or more amino acid residues with one or more non-natural amino acid residues.

In some embodiments, the pharmaceutically acceptable salt is selected from a hydrochloride or acetate salt.

In some embodiments, the composition is encapsulated in or complexed to a nanoparticle. Typically, the nanoparticle is a lipid nanoparticle (e.g., a liposome or lipoplex).

Suitably, the lipid nanoparticle has a diameter of between about 50 nm and 500nm. Typically, the nanoparticle is sized smaller than about 250 nm in diameter (e.g., between about 100 nm to 250 nm).

Typically, the nanoparticle comprises a cationic lipid, a PEG-modified lipid, a sterol, and/or a non-cationic lipid. In some embodiments of this type, the cationic lipid is an ionizable cationic lipid.

In some embodiments, the ionizable cationic lipid is selected from the group comprising or consisting of 1,2-di-O-octadecenyl-3-trimethylammonium propane (DOTMA), N-[1-(2,3-dioleyloxy)propyl]-N,N,N-trimethylammonium chloride (DOTMA), 1,2-Dioleoyl-3-Trimethylammonium-Propane (DOTAP), 5-carboxyspermylglycinedioctadecylamide (DOGS), 2,3-dioleyloxy-N-[2-(spermine-carboxamido)ethyl]-N,N-dimethyl-1-propanaminium (DOSPA), 1,2-Dioleoyl-3-Dimethylammonium-Propane (DODAP), 1,2-distearyloxy-N,N-dimethyl-3-aminopropane (DSDMA), 1,2-dioleyloxy-N,N-dimethyl-3-aminopropane (DODMA), 1,2-dilinoleyloxy-N,N-dimethyl-3-aminopropane (DLinDMA), heptatriaconta-6,9,28,31-tetraen19-yl 4-(dimethylamino)butanoate (DLin-MC3-DMA), 2,2-dilinoleyl-4-(2-dimethylaminoethyl)-[1,3]-dioxolane (DLin-KC2-DMA), 1,2-dilinolenyloxy-N,N-dimethyl-3-aminopropane (DLenDMA), N-dioleyl-N,N-dimethyl ammonium chloride (DODAC), N,N-distearyl-N,N-dimethylammonium bromide (DDAB), N-(1,2-dimyristyloxyprop-3-yl)-N,N-dimethyl-N-hydroxyethyl ammonium bromide (DMRIE), 3-dimethylamino-2-(cholest-5-en-3-beta-oxybutan-4-oxy)-1-(cis, cis-9,12-octadecadienoxy)propane (CLinDMA), 2-[5′-(cholest-5-en-3-beta-oxy)-3′-oxapentoxy)-3-dimethyl-1-(cis,cis-9′,1-2′-octadecadienoxy)propane (CpLinDMA), N,N-dimethyl-3,4-dioleyloxybenzylamine (DMOBA), 1,2-N,N′-dioleylcarbamyl-3-dimethylaminopropane (DOcarbDAP), 2,3-Dilinoleoyloxy-N,N-dimethylpropylamine (DLinDAP), 1,2-N,N′-Dilinoleylcarbamyl-3-dimethylaminopropane (DLincarbDAP), 1,2-Dilinoleoylcarbamyl-3-dimethylaminopropane (DLinCDAP), 2,2-dilinoleyl-4-dimethylaminoethyl-[1,3]-dioxolane (DLin-K-XTC2-DMA), and C12-200. In some embodiments, the nanoparticle comprises a dioleoylphosphatidylethanolamine (DOPE) lipid.

A pharmaceutical composition comprising the PD-L1 bicyclic peptide mimetic as described above and elsewhere herein, in combination with one or more excipients.

A method of reducing nuclear localization of PD-L1 in a PD-L1 overexpressing cell, comprising contacting the cell with a PD-L1 bicyclic peptide mimetic as described above and elsewhere herein.

In another aspect, the present invention provides, methods of treating or preventing cancer in a subject, wherein the cancer comprises at least one PD-L1 overexpressing cell, comprising administering to the subject a PD-L1 bicyclic peptide mimetic as described above and elsewhere herein.

Preferably, the PD-L1 overexpressing cell is a cancer stem cell, a non-cancer stem cell tumour cell.

In some preferred embodiments, the PD-L1 overexpressing cell is a cancer stem cell tumour cell.

In some embodiments, the cancer is selected from breast, prostate, lung, bladder, pancreatic, colon, liver, or brain cancer, or melanoma, or retinoblastoma.

In some embodiments, the methods further comprise administering at least one further cancer therapy. In some embodiments, the further cancer therapy is a chemotherapeutic agent.

### BRIEF DESCRIPTION OF THE FIGURES

FIG. 1 is a representation of the PD-L1 bicycle peptide mimetic structure and sequence. (A) Sequences depict the sequence optimization steps performed to arrive at two candidate peptides. (B) Cartoon representation of a bicyclic peptide candidate, “DL-2”.

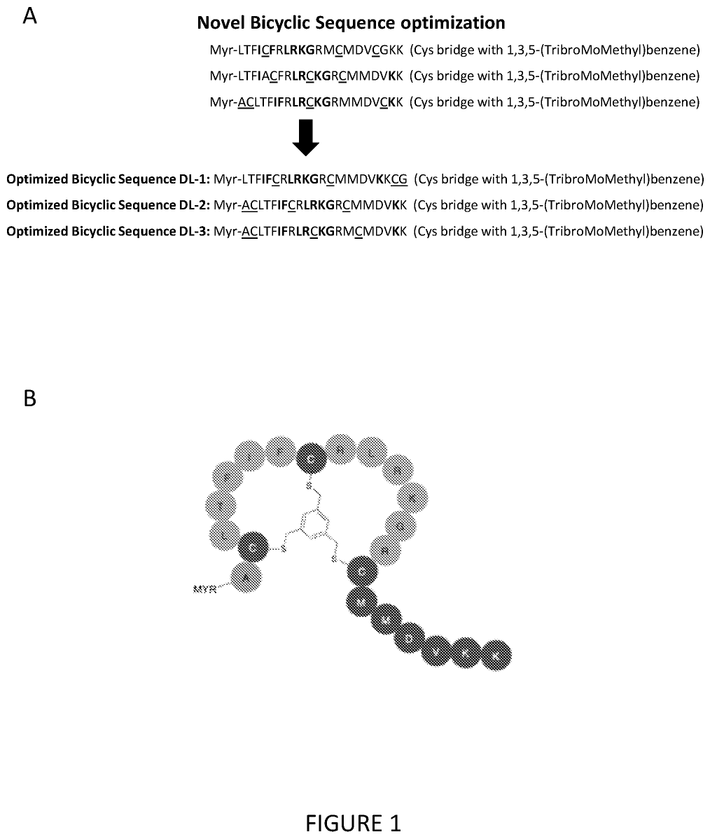

FIG. 2 is a graphical and photographic representation showing the observed PD-L1 PTM in immunotherapy-resistant and responsive cancers. (A) Representative high-resolution immunofluorescence images of PD-L1-PTM expression in immunotherapy-resistant and responsive melanomas and TNBC. (B) Percentage CTC population positive for PD-L1-PTM1 or PTM2 and nuclear fluorescence intensity (NFI) for PTM1 or cytoplasmic fluorescence intensity (CFI) for PTM2 for immunotherapy-resistant and responsive melanomas and TNBC. (C) PD-L1-PTM1 expression is associated with survival in immunotherapy-treated stage IV melanoma patients. Dash lines: PDL1-PTM1 negative; Solid lines: PDL1-PTM1 positive.

FIG. 3 is a photographic representation showing acetylated form of PD-L1 is the nuclear-based variant. (A) An example immunoblot of nuclear or cytoplasmic extracts from MDA-MB-231 or MCF7 breast cancer cell lines with a ponceau red loading control. Immunoblots were probed with our custom antibody to PD-L1-PTM1. (B) Depicts example high through-put image screen demonstrating the nuclear localisation of PD-L1-PTM1 (Ac) or the cytoplasmic/whole cell staining of PD-L1-PTM2 (Me3). Scale bars in bottom right-hand corner indicates 10 μm.

FIG. 4 is a photographic representation confirming the nuclear localization of PD-L1-PTM1 in cancer cell lines. Exemplary super resolution image screen demonstrating the nuclear localisation of PD-L1-PTM1 (Ac) and cytoplasmic localisation of Cytokeratin. Scale bars in bottom right-hand corner indicates 10 μm.

FIG. 5 is a graphical representation showing that dual epigenetic-immunotherapy re-educates and re-programs the T-cell repertoire. (A) Example images of metastatic brain lesions from metastatic TNBC patients stained with antibodies for CSV, PD-L1-PTM1 (PDL1-Ac), PDL1-PTM2 (PDL1-Me3), or a commercial PD-L1 antibody (PDL1-28-8). (B) Bar graphs show fluorescent intensity of PD-L1-28-8, PD-L1-PTM1 or PTM2 along with the percentage of the population positive for CSV and either of these markers with SEM, showing the high throughput image screen data (n≥1000 cells per group).

FIG. 6 is a graphical and photographic representation of infiltrating CD8+ T cells within tumours, and level of PD-L1. Example images of metastatic brain lesions from metastatic TNBC patients stained with antibodies for CD8 and PD-L1-PTM1 (PD-L1-Ac). Bar graphs show the percentage of the population that is positive for CD8 and PD-L1-PTM1 (PD-L1-Ac), showing the high throughput image screen data (n≥1000 cells per group).

FIG. 7 is a graphical representation showing the effect of the PD-L1 bicycle peptide mimetics on cancer cell proliferation. Proliferation assay of MDA-MB-231-Br (MDA-BRM) or MDA-MB-231 TNBC cell lines and the impact of inhibition with DL-1 or DL-2. IC50 values are shown for each assay.

FIG. 8 is a graphical representation showing that PD-L2 bicycle peptide mimetics induce significantly higher cytoplasmic PD-L1-PTM2. Bar graphs show fluorescent intensity of nuclear fluorescent intensity of PD-L1-Ac (PTM1) or cytoplasmic fluorescent intensity of PD-L1-Me3 (PTM2) with SEM, showing the high throughput image screen data (n≥500 cells per group). MDA-MB-231 or MDA-MB-231-Br were treated with either vehicle control or bicyclic inhibitor DL-1 or DL-2 and the expression of PD-L1-Ac (PTM1) within the nuclear or the PD-L1-Me3 (PTM2) within the cytoplasm scored with high resolution digital pathology. Significant differences are calculated as a pair-wise comparison to vehicle control.

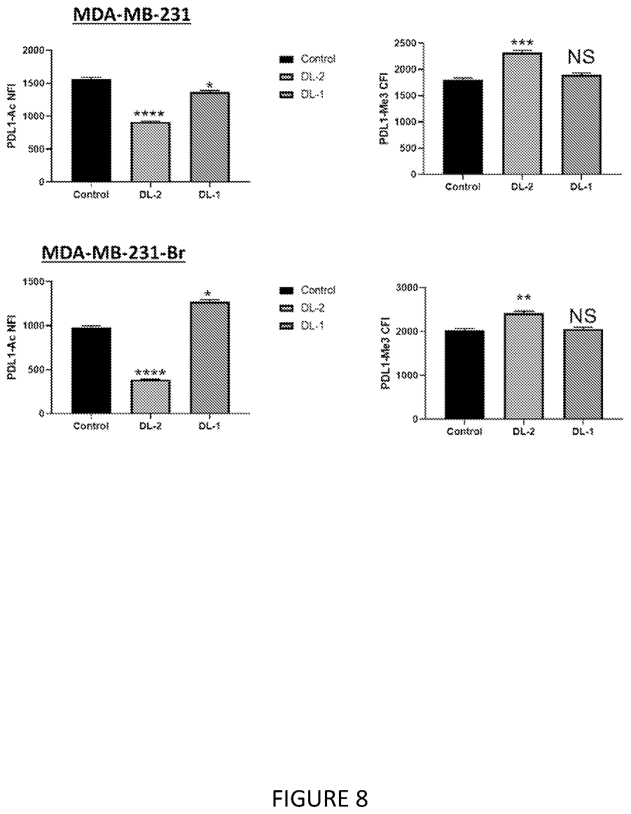

FIG. 9 is a graphical representation showing mesenchymal-regulators of metastatic burden after treatment with the PD-L1 bicycle peptide mimetics. Bar graphs show cytoplasmic or nuclear fluorescent intensity of CSV, ABCB5, EGFR or FOXQ1 with SEM, showing the high throughput image screen data (n≥500 cells per group). MDA-MB-231 or MDA-MB-231-Br were treated with either vehicle control or bicyclic inhibitor DL-1 or DL-2 and the change in expression characterized with high resolution digital pathology. Significant differences are calculated as a pair-wise comparison to vehicle control.

FIG. 10 provides a graphical representation showing that treatment with PD-L1 bicyclic peptide mimetics induce expression of epithelial markers. Bar graphs show cytoplasmic fluorescent intensity of E-Cadherin with SEM, showing the high through-put image screen data (n≥500 cells a group). (A) MDA-MB-231 or (B) MDA-MB-231-Br were treated with either vehicle control or bicyclic peptide inhibitor DL-1 or DL-2 and the change in expression characterized with high resolution digital pathology. Significant differences are calculated as a pair-wise comparison to vehicle control.

FIG. 11 is a photographic and graphical representations showing PD-L1 bicycle peptide mimetics induce upregulation of cell surface PD-L1-Me3. (A) High throughput image screen of non-permeabilized MDA-MB-231 cells treated with vehicle control, HDAC2i or bicyclic peptide inhibitor DL-2. Cells were stained with PD-L1-PTM2 (Me3). Scale bars in bottom right-hand corner of images indicate 10 μm. (B) Graphs plot the mean FI for PD-L1-Me3, n≥100 cells per group. Significant differences are indicated calculated by Kruskal-Wallis non-parametric test.

FIG. 12 provides photographic and graphical representations showing expression levels of nuclear and cytoplasmic PD-L1-Me3 in permeabilized cells. (A) Example high throughput image screen of permeabilized MDA-MB-231 cells treated with vehicle control, HDAC2i or bicyclic peptide inhibitor DL-2. Cells were stained with PDL1-PTM2 (Me3). Scale bars in bottom right-hand corner of images indicate 10 μm. (B) Graphs plot the mean NFI (nuclear FI) or CFI (cytoplasmic FI) for PD-L1-Me3, n≥100 cells per group. Significant differences are indicated calculated by Kruskal-Wallis non-parametric test.

FIG. 13 provides a flowchart outlining the synthesis of an exemplary PD-L1 bicyclic peptide mimetic of the invention.

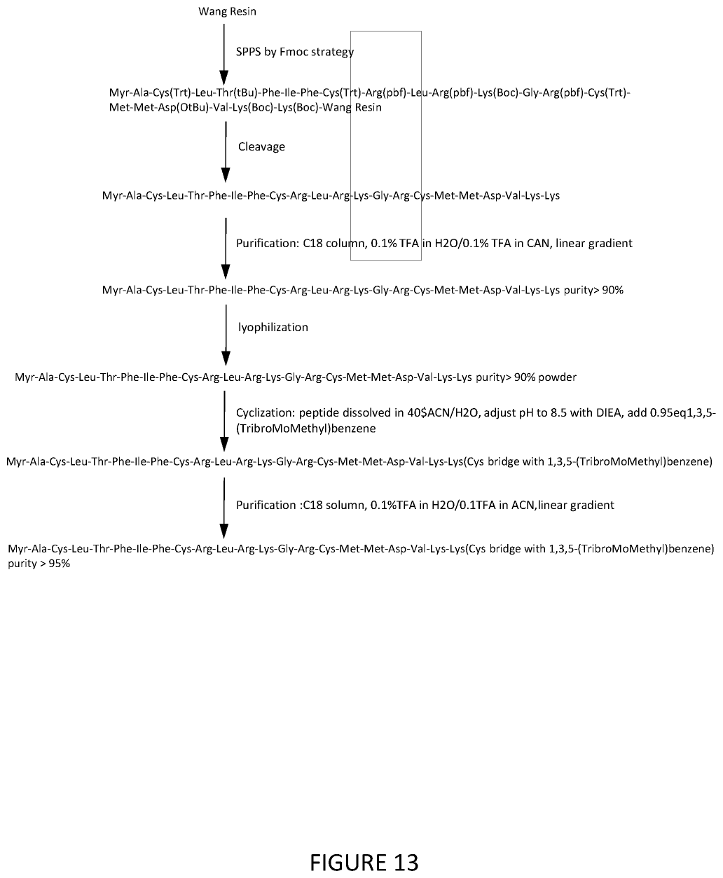

FIG. 14 provides photographic representation showing that bicyclic peptide inhibitor DL-2 inhibits co-localisation of PDL1-PTM1 and IMPα1 but not of IMPα1, or PDL2 and IMPα1. (A) MDA-MB-231 cells were treated with DL-2 for 4 to 96 hours or control peptide scramble and were permeabilised probed a mouse anti-IMPa1, rabbit anti-PDL1 and visualized with a donkey anti-rabbit secondary antibody conjugated to Alexa Fluor 647 or a donkey anti-mouse secondary antibody conjugated to Alexa Fluor 568. Cover slips were mounted on glass microscope slides with ProLong nucblue glass Antifade reagent (Life Technologies). PDL1 and IMPa1 Digital images were analysed using ImageJ software (ImageJ, NIH, Bethesda, MD, USA) to determine the mean Fluorescent Intensity (mean FI). Graphs represent the mean FI of IMPa1. Graphs also depict the PCC of PDL1/IMPa1 using ImageJ software with automatic thresholding and manual selection of regions of interest (ROIs) specific for cell nuclei to calculate the Pearson's co-efficient correlation (PCC) for each pair of antibodies. PCC values range from: −1=inverse of co-localization. (B) High resolution imaging of DUOLINK cells stained with PDL1-Ac & IMPα1.

FIG. 15 provides photographical representation showing that bicyclic peptide DL-2 inhibits the complex of PDL1-PTM1 and IMPα1. High resolution imaging using 100× objective with the ASI digital pathology system of DUOLINK cells stained with PDL1-Ac & IMPα1.

FIG. 16 provides photographic representations showing that the bicyclic peptide DL-2, but not the linear PDL1-L1 peptide, inhibits the complex of PDL1-PTM1 and IMPα1. High resolution imaging using 100× objective with the ASI digital pathology system of DUOLINK cells stained with PDL1-Ac and IMPα1.

FIG. 17 provides photographical representations showing that DL-2 inhibits the complex of PDL1 (unmodified) and IMPα1, but not linear PDL1-L1 peptide inhibitor. High resolution imaging using 100× objective with the ASI digital pathology system of DUOLINK cells stained with PDL1 (unmodified) and IMPα1.

FIG. 18 provides photographical representations showing that DL-2 inhibits the complex of PDL1 (unmodified) and IMPα1 in immunotherapy responsive cancer line CT26.

FIG. 19 provides graphical representations showing that DL-2 bicyclic peptide is specific for the NLS motif, induces PDL1-PTM2 (PDL1-Me3) whereas linear is unable to do so. Bar graphs show cytoplasmic levels of CSV, PDL1-PTM2 with SEM, showing the high throughput image screen data (N≥20 cells a group).

FIG. 20 provide graphical representations showing that DL-2 does not inhibit the complex of PDL2 and IMPα1. High resolution imaging of DUOLINK cells stained with PDL2 and IMPα1. MDA-MB-231 cells were treated with DL-2 or control and were permeabilized and were probed with the DUOLINK ligation assay.

FIG. 21 provides graphical representation illustrating that DL-2 has no effect on nuclear expression of PDL2. High throughput analysis of the specificity of the DL-2 peptide.

FIG. 22 provides graphical representation illustrating that DL-2 has no effect on other nuclear protein expression and is specific for PDL1. Graphs of nuclear intensity of markers PKCθ, LSD1, p65, C-Rel, SET, pSTAT3.

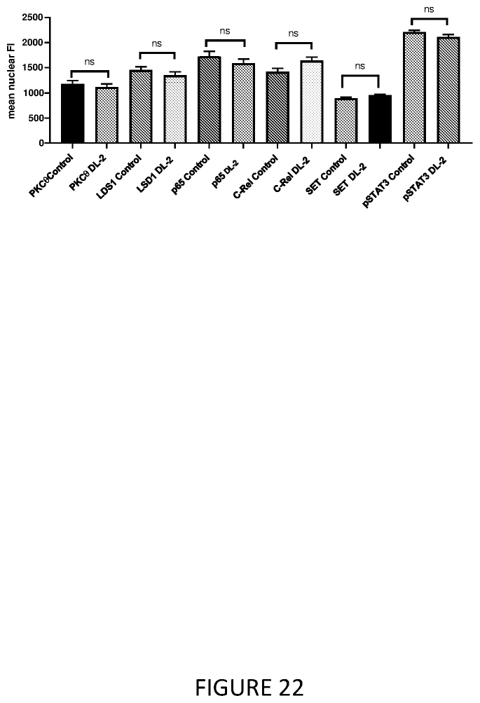

FIG. 23 provides graphical representation showing that the potency of DL-2 is maintained on two different bicyclic scaffolds. (A) CT26, 4T1 or MDA-MB-231 Cells were treated with DL-2 or DL-2-TBAB (two different scaffolds) bicyclic inhibitors for 72 hours. Following inhibition, media was removed and replaced with WST-1 cell proliferation reagent. Absorbance was recorded at 450 nm after 1 hour. Data represent a single independent experiment performed in triplicate, results are graphed as mean +/− standard error (SE). (B) High resolution imaging using 100× objective with the ASI digital pathology system of DUOLINK cells stained with PDL1-Ac (PTM1) and IMPα1. (C) High resolution imaging using 100× objective with the ASI digital pathology system of DUOLINK cells stained with PDL1-unmodified and IMPα1

FIG. 24 provides graphical representation demonstrating the biological stability or DL-2 and DL-2-D. (A) DL-2 and DL-2-D stability was carried out in a rat plasma model. The graph depicts the percentage of the test compound remaining up to 24 hours (1440 minutes). (B) DL-2, DL-2-D, and a linear peptide control was tested in a rat plasma stability model. Graph depicts the percentage of the test compound up to 2 hours.

FIG. 25 shows a graphical representation demonstrating the effect of DL-2-D treatment on cancer cell proliferation. Three cancer cell lines were analysed (A) CT26, (B) 4T1, and (C) MDA-MB-231.

FIG. 26 provides graphical representations showing that DL-2 and DL-2-D effectively inhibit the importin:PDL1 complex.

FIG. 27 shows a graphical representation showing that DL-2 bicyclic peptide variants including DL-2-D induce expression of cytoplasmic PDL1-PTM2 and inhibit CSV. Bar graphs shows level of cytoplasmic of CSV, PDL1-Me3 with SEM, from the high throughput image screen data (N≥20 cells a group).

FIG. 28 provides graphical representations showing that cyclic PDL1 peptide inhibitor failed to inhibit mesenchymal cancer markers. The comparison via high resolution imaging using 100× objective with the Leica Widefield system of cells stained with PDL1-PTM1, CSV and ALDH1A1.

FIG. 29 provides graphical representations showing that DL-2 monotherapy reduces primary tumour volume in the 4T1 TNBC model of metastatic breast cancer. (A) Mice body weight (g) over the 22-day treatment period. (B) Representative images of lungs, livers and spleens harvested from vehicle and DL-2 (30 mg/kg) treated mice at day 22 post inoculation. (C) Final lung, liver and spleen weights at day 22 post inoculation. No statistical differences, one-way ANOVA, Tukey's post test. (D) Tumour volumes of individual mice at day 22 post inoculation. *** p<0.001, versus vehicle at day 22, one-way ANOVA, Tukey's post test. (E) Representative images of tumours from vehicle and DL-2 (30 mg/kg) treated mice harvested at day 22 post inoculation. (F) Tumour weights at day 22 post inoculation. * p<0.05, ** p<0.01 versus vehicle at day 22, one-way ANOVA, Tukey's post-test.

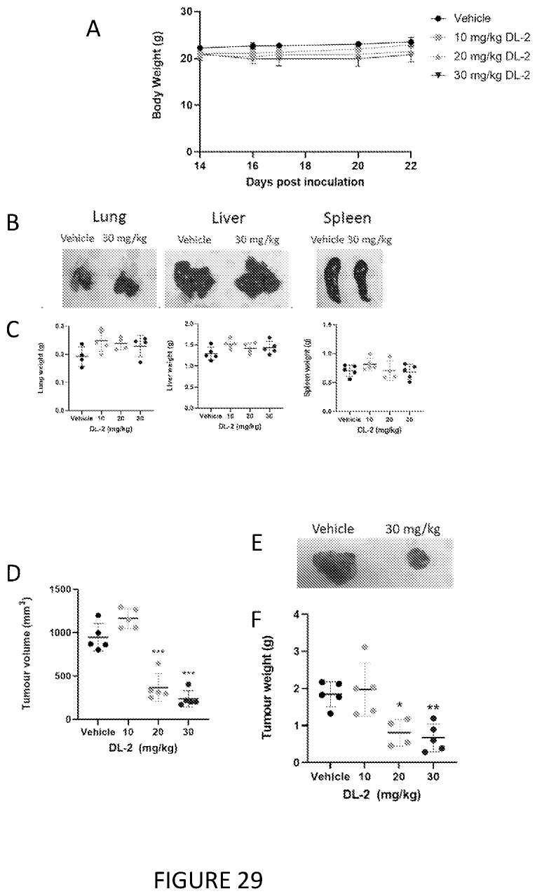

FIG. 30 provides graphical representations showing that DL-2 and αPD1 combination therapy reduces primary tumour volume in the 4T1 model of metastatic breast cancer. (A) Mouse body weight (g) over the 10 day treatment period. (B) Final lung, liver and spleen weights at day 19 post inoculation. No statistical differences, one-way ANOVA, Tukey's post test. (C) Tumour volumes of mice treated with vehicle (saline) or DL-2 (10 mg/kg equivalent) in conjunction with αPD1 or isotype control (10 mg/kg) (n=5 per group). (D) Tumour volumes of individual mice at day 19 post inoculation (data also represented in C). * p<0.05, ** p<0.01, versus vehicle+isotype at day 19, one-way ANOVA, Tukey's post test. (E) Representative images of tumours harvested at day 19 post inoculation.

FIG. 31 provides graphical representations showing that a DL-2-D and αPD1 combination therapy reduces primary tumour burden and lung metastasis in the 4T1 model of metastatic breast cancer. (A) Mouse body weight (g) over 20-day treatment period. (B) Final lung, liver and spleen weights at day 20 post inoculation. (C) Tumour volumes of mice treated with vehicle (saline) or DL-2-D (20 mg/kg) in conjunction with αPD1 or isotype control (10 mg/kg) (n=5 per group). (D) Tumour volumes of individual mice at day 20 post inoculation (data also represented in C. * p<0.05, DL-2-D+αPD1 versus vehicle+αPD1 and ** p<0.01, versus vehicle+isotype at day 20, Mann-Whitney post test. (E) Representative images of tumours harvested at day 20 post inoculation. (F) Lung nodule counts in mice treated with vehicle (saline) or DL-2-D (20 mg/kg) in conjunction with αPD1 or isotype control (10 mg/kg) (n=5 per group) at day 20 post inoculation. Lungs were fixed in Bouin's solution and examined for the presence of surface metastases. * p<0.05, DL-2-D+αPD1 versus vehicle+isotype and ** p<0.01, versus DL-2-D+isotype at day 20, Mann-Whitney post test.

FIG. 32 provides graphical representations showing a comparison between DL-2 and DL-2-D. (A) As per FIGS. 24, and 25, above. (B) Fixed cells were washed with PBS and resuspended in PBS containing 1% BSA prior to antibody staining. Primary antibodies targeting CD8, TIM3 and PD1 antibodies were used. Appropriate antibody controls were used for all flow cytometry stain and acquisition. All samples were acquired on an LSR Fortessa cytometer (BD Biosciences, Franklin Lakes, NJ), and data were analyzed using FlowJo v10 software

FIG. 33 provides a graphical representation of DL-2 inhibits resistance signature in pre-clinical treatment of MICs from metastatic immunotherapy resistant cancers. Graph represents the CFI values for CSV, NFI for PDL1-PTM1, ALDH1A1 and FI for ABCB5 measured using the ASI Digital pathology automated system to select the nucleus minus background (n≥40 cells/sample/5 patients a group). The Mann-Whitney non-parametric t-test was used for pairwise comparisons and the Kruskal-Wallis to compare groups where ****p<0.0001, ***p<0.001, **p<0.01, and *p<0.05.

FIG. 34 provides graphical representation of DL-2-N purification. (A) Size distribution graph of control formulation (i.e., hollow nanoparticle). (B) Size distribution graphs of DL-2 nanoparticle (DL-2-NP) formulation. Nanoparticles were formed by combination of lipid nanoparticles with DL-2 at a ratio of 3:1 at a flow rate of 12 mL/min to 18 mL/min, or with just lipid nanoparticle for hollow control. Intensity of nanoparticle size was determined using a DLS which is a photon correlation spectroscopy—using light scattering to measure the Brownian motion of the particles. (C) DL-2, DL-2-NP, and DL-2-D stability was carried out in a rat Plasma model. The graph shows the percentage of the test compound remaining after 24 hours (1440 minutes). Blue plot: DL-2; orange plot: DL2-D; and grey plot: DL-2-NP.

FIG. 35 provides graphical and photographical representations of the effect of DL-2-NP treatment on cell proliferation using MDA-MB-231 cancer cell lines. (A) Data represent a single independent experiment performed in triplicate; results are graphed as mean +/− standard error (SE). Concentration represents ten concentration readings from 1=0.001 nM to 10=10 nM. (B) MD A-MB-231 cells were treated with DL-2 or vehicle control for 24 hours, with concentrations ranging from 10 nM to 0.3 nM. High resolution imaging of MDA-MB-231 cells stained with PDL1-PTM1 (PDL1-Ac), CSV, or DLL4. Scale bar indicates 10 μm. (B) graphs represent the mean fluorescent intensity in the nucleus (NFI), the cytoplasm (CFI) compartments or the ratio of nuclear to fluorescent staining (Fn/c), wherein a ratio of greater than 1 means nuclear bias, less than 1 means cytoplasmic bias and 0 means equal distribution. Significant differences were calculated as per Kruskal-Wallis one-way ANOVA.

FIG. 36 provides a graphical representation demonstrating that DL-2-NP effectively inhibits target complex of PDL1 and Imp□. (A, B) Comparison via high resolution imaging using 100× objective with the ASI digital pathology system of DUOLINK cells stained with PDL1-unmodified & IMPα1. (A) PDL1 (unmodified) and IMPα1 DUOLINK digital images were analysed using ImageJ software (ImageJ, NIH, Bethesda, MD, USA). (B) Graphs represent the mean DOT Fluorescent Intensity in the nucleus or the cytoplasm compartments with significant differences calculated as per one-way ANOVA comparison. The red cut-off represents the IC50 for DL-2-NP for abrogating expression for the PDL1-(unmodified) and IMPα1 complex. (C, D) MDA-MB-231 cells were treated with vehicle control (hollow nanoparticle) or DL-2-NP at a concentration of 10 nM to 0.3 nM. (C) Comparison via high resolution imaging using 100× objective with the ASI digital pathology system of DUOLINK cells stained with PDL1-PTM1 and IMPα1. Cells were permeabilised and were probed with the DUOLINK ligation assay and PDL1-PTM1 and IMPα1 DUOLINK Digital images were analysed using ImageJ software (ImageJ, NIH, Bethesda, MD, USA). (B) graphs represent the mean DOT Fluorescent Intensity in the nucleus or the cytoplasm compartments with significant differences calculated as per one-way ANOVA comparison. The red cut-off represents the IC50 for DL-2-NP for abrogating expression for the PDL1-PTM1 and IMPα1.

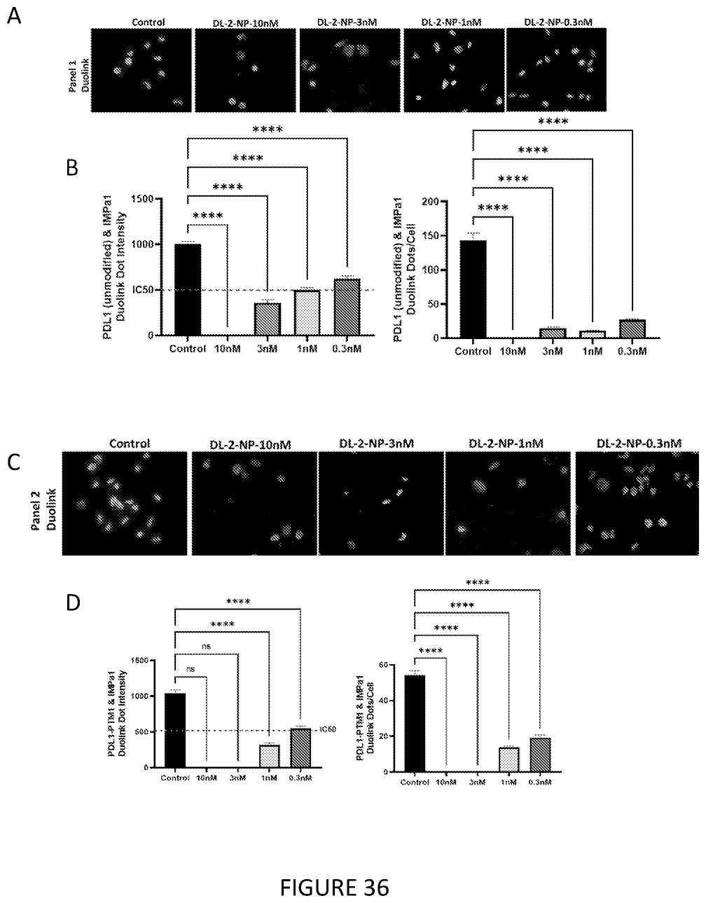

### DETAILED DESCRIPTION OF THE INVENTION

### 1. DEFINITIONS

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e., to at least one) of the grammatical object of the article. By way of example, “an element” means one element or more than one element.

The term “about” as used herein refers to the usual error range for the respective value readily known to the skilled person in this technical field. Reference to “about” a value or parameter herein includes (and describes) embodiments that are directed to that value or parameter per se.

The term “acetylation site” is used herein to refer to any amino acid sequence that may be acetylated, for example, by an acetyltransferase; especially a histone acetyltransferase, non-limiting examples of which include GCN5, Hatl, ATF-2, Tip60, MOZ, MORF, HBO1, p300, CBP, SRC-1, ACTR, TIF-2, SRC-3, TAF1, TFIIIC and/or CLOCK, most especially p300. The term “acetylation site” refers to a sequence comprising an acetylation substrate, such as a lysine residue, and surrounding and/or proximal amino acid residues which may be involved in substrate recognition by an enzyme, such as an acetyltransferase. The acetylation site may be an amino acid sequence of any suitable length, such as, 5, 6, 7, 8, 9, 10, 11, 12, 13, 14, 15, 16, 17, 18, 19, 20, 21, 22 or greater than 22 residues in length, preferably 14, 15, 16, 17, 18, 19, 20 or 21 residues in length.

The term “agent” includes a compound that induces a desired pharmacological and/or physiological effect. The term also encompasses pharmaceutically acceptable and pharmacologically active ingredients of those compounds specifically mentioned herein including but not limited to salts, esters, amides, prodrugs, active metabolites, analogs and the like. When the above term is used, then it is to be understood that this includes the active agent per se as well as pharmaceutically acceptable, pharmacologically active salts, esters, amides, prodrugs, metabolites, analogs, etc. The term “agent” is not to be construed narrowly but extends to small molecules, PD-L1 bicyclic peptide mimetics such as peptides, polypeptides, and proteins as well as compositions comprising them and genetic molecules such as RNA, DNA and mimetics and chemical analogs thereof as well as cellular agents.

As used herein, “and/or” refers to and encompasses any and all possible combinations of one or more of the associated listed items, as well as the lack of combinations when interpreted in the alternative (or).

The term “cancer stem cell” (CSC) refers to a cell that has tumour-initiating and tumour-sustaining capacity, including the ability to extensively proliferate, form new tumours and maintain cancer development, i.e., cells with indefinite proliferative potential that drive the formation and growth of tumours. CSCs are biologically distinct from the bulk tumour cells and possess characteristics associated with stem cells, specifically the ability to self renew and to propagate and give rise to all cell types found in a particular cancer sample. The term “cancer stem cell” includes both gene alteration in stem cells (SCs) and gene alteration in a cell which becomes a CSC. In specific embodiments, the CSCs are breast CSCs, which are suitably CD24+CD44+, illustrative examples of which include CD44^{high} CD24^{low}.

The terms “cancer” and “cancerous” refer to or describe the physiological condition in subjects that is typically characterized by unregulated cell growth. Examples of cancer include but are not limited to, carcinoma, lymphoma, blastoma, sarcoma, and leukemia or lymphoid malignancies. More particular examples of such cancers include, but not limited to, squamous cell cancer (e.g., epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer and gastrointestinal stromal cancer, pancreatic cancer, glioblastoma, cervical cancer, ovarian cancer, liver cancer, bladder cancer, cancer of the urinary tract, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, melanoma, superficial spreading melanoma, lentigo maligna melanoma, acral lentiginous melanomas, nodular melanomas, multiple myeloma and B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); hairy cell leukemia; chronic myeloblastic leukemia; and post-transplant lymphoproliferative disorder (PTLD), as well as abnormal vascular proliferation associated with phakomatoses, edema (such as that associated with brain tumours), Meigs' syndrome, brain, as well as head and neck cancer, and associated metastases. In certain embodiments, cancers that are amenable to treatment by the antibodies of the invention include breast cancer, colorectal cancer, rectal cancer, non-small cell lung cancer, glioblastoma, non-Hodgkin's lymphoma (NHL), renal cell cancer, prostate cancer, liver cancer, pancreatic cancer, soft-tissue sarcoma, Kaposi's sarcoma, carcinoid carcinoma, head and neck cancer, ovarian cancer, mesothelioma, and multiple myeloma. In some embodiments, the cancer is selected from: small cell lung cancer, glioblastoma, neuroblastomas, melanoma, breast carcinoma, gastric cancer, colorectal cancer (CRC), and hepatocellular carcinoma. Yet, in some embodiments, the cancer is selected from: non-small cell lung cancer, colorectal cancer, glioblastoma and breast carcinoma, including metastatic forms of those cancers. In specific embodiments, the cancer is melanoma or lung cancer, suitably metastatic melanoma or metastatic lung cancer.

“Chemotherapeutic agent” includes compounds useful in the treatment of cancer. Examples of chemotherapeutic agents include erlotinib (TARCEVA®, Genentech/OSI Pharm.), bortezomib (VELCADE®, Millennium Pharm.), disulfiram, epigallocatechin gallate, salinosporamide A, carfilzomib, 17-AAG (geldanamycin), radicicol, lactate dehydrogenase A (LDH-A), fulvestrant (FASLODEX®, AstraZeneca), sunitib (SUTENT®, Pfizer/Sugen), letrozole (FEMARA®, Novartis), imatinib mesylate (GLEEVEC®, Novartis), finasunate (VATALANIB®, Novartis), oxaliplatin (ELOXATIN®, Sanofi), 5-FU (5-fluorouracil), leucovorin, Rapamycin (Sirolimus, RAPAMUNE®, Wyeth), Lapatinib (TYKERB®, GSK572016, Glaxo Smith Kline), Lonafamib (SCH 66336), sorafenib (NEXAVAR®, Bayer Labs), gefitinib (IRESSA®, AstraZeneca), AG1478, alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including topotecan and irinotecan); bryostatin; callystatin; CC-1065 (including its adozelesin, carzelesin and bizelesin synthetic analogs); cryptophycins (particularly cryptophycin 1 and cryptophycin 8); adrenocorticosteroids (including prednisone and prednisolone); cyproterone acetate; 5α-reductases including finasteride and dutasteride); vorinostat, romidepsin, panobinostat, valproic acid, mocetinostat dolastatin; aldesleukin, talc duocarmycin (including the synthetic analogs, KW-2189 and CB1-TM1); eleutherobin; pancratistatin; a sarcodictyin; spongistatin; nitrogen mustards such as chlorambucil, chlomaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g., calicheamicin, especially calicheamicin γ1I and calicheamicin ω1I (Angew Chem. Intl. Ed. Engl. 1994 33:183-186); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® (doxorubicin), morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone; etoglucid; gallium nitrate; hydroxyurea; lentinan; lonidainine; maytansinoids such as maytansine and ansamitocins; mitoguazone; mitoxantrone; mopidamnol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids (e.g., TAXOL (paclitaxel; Bristol-Myers Squibb Oncology, Princeton, N.J.), ABRAXANE® (Cremophor-free), albumin-engineered nanoparticle formulations of paclitaxel (American Pharmaceutical Partners, Schaumberg, Ill.), and TAXOTERE® (docetaxel, doxetaxel; Sanofi-Aventis)); chloranmbucil; GEMZAR® (gemcitabine); 6-thioguanine; mercaptopurine; methotrexate; platinum analogs such as cisplatin and carboplatin; vinblastine; etoposide (VP-16); ifosfamide; mitoxantrone; vincristine; NAVELBINE® (vinorelbine); novantrone; teniposide; edatrexate; daunomycin; aminopterin; capecitabine (XELODA®); ibandronate; CPT-11; topoisomerase inhibitor RFS 2000; difluoromethylornithine (DMFO); retinoids such as retinoic acid; and pharmaceutically acceptable salts, acids and derivatives of any of the above.

Chemotherapeutic agent also includes (i) anti-hormonal agents that act to regulate or inhibit hormone action on tumours such as anti-estrogens and selective estrogen receptor modulators (SERMs), including, for example, tamoxifen (including NOLVADEX®; tamoxifen citrate), raloxifene, droloxifene, iodoxyfene, 4-hydroxytamoxifen, trioxifene, keoxifene, LY117018, onapristone, and FARESTON® (toremifine citrate); (ii) aromatase inhibitors that inhibit the enzyme aromatase, which regulates estrogen production in the adrenal glands, such as, for example, 4(5)-imidazoles, aminoglutethimide, MEGASE® (megestrol acetate), AROMASIN® (exemestane; Pfizer), formestanie, fadrozole, RIVISOR® (vorozole), FEMARA® (letrozole; Novartis), and ARIMIDEX® (anastrozole; AstraZeneca); (iii) anti-androgens such as flutamide, nilutamide, bicalutamide, leuprolide and goserelin; buserelin, tripterelin, medroxyprogesterone acetate, diethylstilbestrol, premarin, fluoxymesterone, all transretionic acid, fenretinide, as well as troxacitabine (a 1,3-dioxolane nucleoside cytosine analog); (iv) protein kinase inhibitors; (v) lipid kinase inhibitors; (vi) antisense oligonucleotides, particularly those which inhibit expression of genes in signaling pathways implicated in aberrant cell proliferation, such as, for example, PKC-α, Ralf and H-Ras; (vii) ribozymes such as VEGF expression inhibitors (e.g., ANGIOZYME®) and HER2 expression inhibitors; (viii) vaccines such as gene therapy vaccines, for example, ALLOVECTIN®, LEUVECTIN®, and VAXID®; PROLEUKIN®, rIL-2; a topoisomerase 1 inhibitor such as LURTOTECAN®; ABARELIX® rmRH; and (ix) pharmaceutically acceptable salts, acids and derivatives of any of the above.

Chemotherapeutic agent also includes antibodies such as alemtuzumab (Campath), bevacizumab (AVASTIN®, Genentech); cetuximab (ERBITUX®, Imclone); panitumumab (VECTIBIX®, Amgen), rituximab (RITUXAN®, Genentech/Biogen Idec), pertuzumab (OMNITARG®, 2C4, Genentech), trastuzumab (HERCEPTIN®, Genentech), tositumomab (Bexxar, Corixia), and the antibody drug conjugate, gemtuzumab ozogamicin (MYLOTARG®, Wyeth). Additional humanized monoclonal antibodies with therapeutic potential as agents in combination with the compounds of the invention include: apolizumab, aselizumab, atlizumab, bapineuzumab, bivatuzumab mertansine, cantuzumab mertansine, cedelizumab, certolizumab pegol, cidfusituzumab, cidtuzumab, daclizumab, eculizumab, efalizumab, epratuzumab, erlizumab, felvizumab, fontolizumab, gemtuzumab ozogamicin, inotuzumab ozogamicin, ipilimumab, labetuzumab, lintuzumab, matuzumab, mepolizumab, motavizumab, motovizumab, natalizumab, nimotuzumab, nolovizumab, numavizumab, ocrelizumab, omalizumab, palivizumab, pascolizumab, pecfusituzumab, pectuzumab, pexelizumab, ralivizumab, ranibizumab, reslivizumab, reslizumab, resyvizumab, rovelizumab, ruplizumab, sibrotuzumab, siplizumab, sontuzumab, tacatuzumab tetraxetan, tadocizumab, talizumab, tefibazumab, tocilizumab, toralizumab, tucotuzumab celmoleukin, tucusituzumab, umavizumab, urtoxazumab, ustekinumab, visilizumab, and the anti-interleukin-12 (ABT-874/J695, Wyeth Research and Abbott Laboratories) which is a recombinant exclusively human-sequence, full-length IgG₁λ antibody genetically modified to recognize interleukin-12 p40 protein.

Chemotherapeutic agent also includes “EGFR inhibitors,” which refers to compounds that bind to or otherwise interact directly with EGFR and prevent or reduce its signaling activity, and is alternatively referred to as an “EGFR antagonist.” Examples of such agents include antibodies and small molecules that bind to EGFR. Examples of antibodies which bind to EGFR include MAb 579 (ATCC CRL HB 8506), MAb 455 (ATCC CRL HB8507), MAb 225 (ATCC CRL 8508), MAb 528 (ATCC CRL 8509) (see, U.S. Pat. No. 4,943,533, Mendelsohn et al.) and variants thereof, such as chimerized 225 (C225 or Cetuximab; ERBUTIX®) and reshaped human 225 (H225) (see, WO 96/40210, Imclone Systems Inc.); IMC-11F8, a fully human, EGFR-targeted antibody (Imclone); antibodies that bind type II mutant EGFR (U.S. Pat. No. 5,212,290); humanized and chimeric antibodies that bind EGFR as described in U.S. Pat. No. 5,891,996; and human antibodies that bind EGFR, such as ABX-EGF or Panitumumab (see WO98/50433, Abgenix/Amgen); EMD 55900 (Stragliotto et al. Eur. J. Cancer 32A:636-640 (1996)); EMD7200 (matuzumab) a humanized EGFR antibody directed against EGFR that competes with both EGF and TGF-α for EGFR binding (EMD/Merck); human EGFR antibody, HuMax-EGFR (GenMab); fully human antibodies known as E1.1, E2.4, E2.5, E6.2, E6.4, E2.11, E6. 3 and E7.6. 3 and described in U.S. Pat. No. 6,235,883; MDX-447 (Medarex Inc); and mAb 806 or humanized mAb 806 (Johns et al., J. Biol. Chem. 279(29):30375-30384 (2004)). The anti-EGFR antibody may be conjugated with a cytotoxic agent, thus generating an immunoconjugate (see, e.g., EP659439A2, Merck Patent GmbH). EGFR antagonists include small molecules such as compounds described in U.S. Pat. Nos. 5,616,582, 5,457,105, 5,475,001, 5,654,307, 5,679,683, 6,084,095, 6,265,410, 6,455,534, 6,521,620, 6,596,726, 6,713,484, 5,770,599, 6,140,332, 5,866,572, 6,399,602, 6,344,459, 6,602,863, 6,391,874, 6,344,455, 5,760,041, 6,002,008, and 5,747,498, as well as the following PCT publications: WO98/14451, WO98/50038, WO99/09016, and WO99/24037. Particular small molecule EGFR antagonists include OSI-774 (CP-358774, erlotinib, TARCEVA® Genentech/OSI Pharmaceuticals); PD 183805 (CI 1033, 2-propenamide, N-[4-[(3-chloro-4-fluorophenyl)amino]-7-[3-(4-morpholinyl)propoxy]-6-quin-azolinyl]-, dihydrochloride, Pfizer Inc.); ZD1839, gefitinib (IRESSA®) 4-(3′-Chloro-4′-fluoroanilino)-7-methoxy-6-(3-morpholinopropoxy)quinazoline, AstraZeneca); ZM 105180 ((6-amino-4-(3-methylphenyl-amino)-quinazoline, Zeneca); BIBX-1382 (N8-(3-chloro-4-fluoro-phenyl)-N2-(1-methyl-piperidin-4-yl)-pyrimido[5,4-d]pyrimidine-2,8-diamine, Boehringer Ingelheim); PKI-166 ((R)-4-[4-[(1-phenylethyl)amino]-1H-pyrrolo[2,3-d]pyrimidin-6-yl]-phenol)- ; (R)-6-(4-hydroxyphenyl)-4-[(1-phenylethyl)amino]-7H-pyrrolo[2,3-d]pyrimi-dine); CL-387785 (N-[4-[(3-bromophenyl)amino]-6-quinazolinyl]-2-butynamide); EKB-569 (N-[4-[(3-chloro-4-fluorophenyl)amino]-3-cyano-7-ethoxy-6-quinolinyl]-4-(-dimethylamino)-2-butenamide) (Wyeth); AG1478 (Pfizer); AG1571 (SU 5271; Pfizer); dual EGFR/HER2 tyrosine kinase inhibitors such as lapatinib (TYKERB®, GSK572016 or N-[3-chloro-4-[(3 fluorophenyl)methoxy]phenyl]-6[5[[[2methylsulfonypethyl]amino]methyl]-2-furanyl]-4-quinazolinamine).

Chemotherapeutic agents also include “tyrosine kinase inhibitors” including the EGFR-targeted drugs noted in the preceding paragraph; small molecule HER2 tyrosine kinase inhibitor such as TAK165 available from Takeda; CP-724,714, an oral selective inhibitor of the ErbB2 receptor tyrosine kinase (Pfizer and OSI); dual-HER inhibitors such as EKB-569 (available from Wyeth) which preferentially binds EGFR but inhibits both HER2 and EGFR-overexpressing cells; lapatinib (GSK572016; available from Glaxo-SmithKline), an oral HER2 and EGFR tyrosine kinase inhibitor; PKI-166 (available from Novartis); pan-HER inhibitors such as canertinib (CI-1033; Pharmacia); Raf-1 inhibitors such as antisense agent ISIS-5132 available from ISIS Pharmaceuticals which inhibit Raf-1 signaling; non-HER targeted TK inhibitors such as imatinib mesylate (GLEEVEC®, available from Glaxo SmithKline); multi-targeted tyrosine kinase inhibitors such as sunitinib (SUTENT®, available from Pfizer); VEGF receptor tyrosine kinase inhibitors such as vatalanib (PTK787/ZK222584, available from Novartis/Schering AG); MAPK extracellular regulated kinase I inhibitor CI-1040 (available from Pharmacia); quinazolines, such as PD 153035,4-(3-chloroanilino) quinazoline; pyridopyrimidines; pyrimidopyrimidines; pyrrolopyrimidines, such as CGP 59326, CGP 60261 and CGP 62706; pyrazolopyrimidines, 4-(phenylamino)-7H-pyrrolo[2,3-d] pyrimidines; curcumin (diferuloyl methane, 4,5-bis (4-fluoroanilino)phthalimide); tyrphostines containing nitrothiophene moieties; PD-0183805 (Warner-Lamber); antisense molecules (e.g., those that bind to HER-encoding nucleic acid); quinoxalines (U.S. Pat. No. 5,804,396); tryphostins (U.S. Pat. No. 5,804,396); ZD6474 (Astra Zeneca); PTK-787 (Novartis/Schering AG); pan-HER inhibitors such as CI-1033 (Pfizer); Affinitac (ISIS 3521; Isis/Lilly); imatinib mesylate (GLEEVEC®); PKI 166 (Novartis); GW2016 (Glaxo SmithKline); CI-1033 (Pfizer); EKB-569 (Wyeth); Semaxinib (Pfizer); ZD6474 (AstraZeneca); PTK-787 (Novartis/Schering AG); INC-1C11 (Imclone), rapamycin (sirolimus, RAPAMUNE®); or as described in any of the following patent publications: U.S. Pat. No. 5,804,396; WO 1999/09016 (American Cyanamid); WO 1998/43960 (American Cyanamid); WO 1997/38983 (Warner Lambert); WO 1999/06378 (Warner Lambert); WO 1999/06396 (Warner Lambert); WO 1996/30347 (Pfizer, Inc); WO 1996/33978 (Zeneca); WO 1996/3397 (Zeneca) and WO 1996/33980 (Zeneca).

Chemotherapeutic agents also include dexamethasone, interferons, colchicine, metoprine, cyclosporine, amphotericin, metronidazole, alemtuzumab, alitretinoin, allopurinol, amifostine, arsenic trioxide, asparaginase, BCG live, bevacuzimab, bexarotene, cladribine, clofarabine, darbepoetin alfa, denileukin, dexrazoxane, epoetin alfa, elotinib, filgrastim, histrelin acetate, ibritumomab, interferon alfa-2a, interferon alfa-2b, lenalidomide, levamisole, mesna, methoxsalen, nandrolone, nelarabine, nofetumomab, oprelvekin, palifermin, pamidronate, pegademase, pegaspargase, pegfilgrastim, pemetrexed disodium, plicamycin, porfimer sodium, quinacrine, rasburicase, sargramostim, temozolomide, VM-26, 6-TG, toremifene, tretinoin, ATRA, valrubicin, zoledronate, and zoledronic acid, and pharmaceutically acceptable salts thereof.

Chemotherapeutic agents also include hydrocortisone, hydrocortisone acetate, cortisone acetate, tixocortol pivalate, triamcinolone acetonide, triamcinolone alcohol, mometasone, amcinonide, budesonide, desonide, fluocinonide, fluocinolone acetonide, betamethasone, betamethasone sodium phosphate, dexamethasone, dexamethasone sodium phosphate, fluocortolone, hydrocortisone-17-butyrate, hydrocortisone-17-valerate, aclometasone dipropionate, betamethasone valerate, betamethasone dipropionate, prednicarbate, clobetasone-17-butyrate, clobetasol-17-propionate, fluocortolone caproate, fluocortolone pivalate and fluprednidene acetate; immune selective anti-inflammatory peptides (ImSAIDs) such as phenylalanine-glutamine-glycine (FEG) and its D-isomeric form (feG) (IMULAN BioTherapeutics, LLC); anti-rheumatic drugs such as azathioprine, ciclosporin (cyclosporine A), D-penicillamine, gold salts, hydroxychloroquine, leflunomideminocycline, sulfasalazine, tumour necrosis factor α (TNF-α) blockers such as etanercept (Enbrel), infliximab (Remicade), adalimumab (Humira), certolizumab pegol (Cimzia), golimumab (Simponi), Interleukin 1 (IL-1) blockers such as anakinra (Kineret), T-cell costimulation blockers such as abatacept (Orencia), Interleukin 6 (IL-6) blockers such as tocilizumab (ACTEMERA®); Interleukin 13 (IL-13) blockers such as lebrikizumab; Interferon α (IFN) blockers such as Rontalizumab; Beta 7 integrin blockers such as rhuMAb Beta7; IgE pathway blockers such as Anti-M1 prime; Secreted homotrimeric LTa3 and membrane bound heterotrimer LTa1/β2 blockers such as Anti-lymphotoxin α (LTa); radioactive isotopes (e.g., At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu); miscellaneous investigational agents such as thioplatin, PS-341, phenylbutyrate, ET-18-OCH₃, or farnesyl transferase inhibitors (L-739749, L-744832); polyphenols such as quercetin, resveratrol, piceatannol, epigallocatechine gallate, theaflavins, flavanols, procyanidins, betulinic acid and derivatives thereof; autophagy inhibitors such as chloroquine; delta-9-tetrahydrocannabinol (dronabinol, MARINOL®); beta-lapachone; lapachol; colchicines; betulinic acid; acetylcamptothecin, scopolectin, and 9-aminocamptothecin); podophyllotoxin; tegafur (UFTORAL®); bexarotene (TARGRETIN®); bisphosphonates such as clodronate (for example, BONEFOS® or OSTAC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTONEL®); and epidermal growth factor receptor (EGF-R); vaccines such as THERATOPE® vaccine; perifosine, COX-2 inhibitor (e.g. celecoxib or etoricoxib), proteosome inhibitor (e.g. PS341); CCI-779; tipifarnib (R11577); orafenib, ABT510; Bcl-2 inhibitor such as oblimersen sodium (GENASENSE®); pixantrone; farnesyltransferase inhibitors such as lonafarnib (SCH 6636, SARASAR™); and pharmaceutically acceptable salts, acids or derivatives of any of the above; as well as combinations of two or more of the above such as CHOP, an abbreviation for a combined therapy of cyclophosphamide, doxorubicin, vincristine, and prednisolone; and FOLFOX, an abbreviation for a treatment regimen with oxaliplatin (ELOXATIN™) combined with 5-FU and leucovorin.

Chemotherapeutic agents also include non-steroidal anti-inflammatory drugs with analgesic, antipyretic and anti-inflammatory effects. NSAIDs include non-selective inhibitors of the enzyme cyclooxygenase. Specific examples of NSAIDs include aspirin, propionic acid derivatives such as ibuprofen, fenoprofen, ketoprofen, flurbiprofen, oxaprozin and naproxen, acetic acid derivatives such as indomethacin, sulindac, etodolac, diclofenac, enolic acid derivatives such as piroxicam, meloxicam, tenoxicam, droxicam, lornoxicam and isoxicam, fenamic acid derivatives such as mefenamic acid, meclofenamic acid, flufenamic acid, tolfenamic acid, and COX-2 inhibitors such as celecoxib, etoricoxib, lumiracoxib, parecoxib, rofecoxib, rofecoxib, and valdecoxib. NSAIDs can be indicated for the symptomatic relief of conditions such as rheumatoid arthritis, osteoarthritis, inflammatory arthropathies, ankylosing spondylitis, psoriatic arthritis, Reiter's syndrome, acute gout, dysmenorrhoea, metastatic bone pain, headache and migraine, postoperative pain, mild-to-moderate pain due to inflammation and tissue injury, pyrexia, ileus, and renal colic.

Throughout this specification, unless the context requires otherwise, the words “comprise,” “comprises” and “comprising” will be understood to imply the inclusion of a stated step or element or group of steps or elements but not the exclusion of any other step or element or group of steps or elements. Thus, use of the term “comprising” and the like indicates that the listed elements are required or mandatory, but that other elements are optional and may or may not be present. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

By “corresponds to” or “corresponding to” is meant an amino acid sequence that displays substantial sequence similarity or identity to a reference amino acid sequence. In general the amino acid sequence will display at least about 70, 71, 72, 73, 74, 75, 76, 77, 78, 79, 80, 81, 82, 83, 84, 85, 86, 97, 88, 89, 90, 91, 92, 93, 94, 95, 96, 97, 98, 99% or even up to 100% sequence similarity or identity to at least a portion of the reference amino acid sequence.

By “derivative” is meant a molecule, such as a polypeptide, that has been derived from the basic molecule by modification, for example by conjugation or complexing with other chemical moieties or by post-translational modification techniques as would be understood in the art. The term “derivative” also includes within its scope alterations that have been made to a parent sequence including additions or deletions that provide for functionally equivalent molecules.

As used herein, the term “dosage unit form” refers to physically discrete units suited as unitary dosages for the subject to be treated, each unit containing a predetermined quantity of active material calculated to produce the desired therapeutic effect in association with the required pharmaceutically acceptable vehicle.

An “effective amount” is at least the minimum amount required to effect a measurable improvement or prevention of a particular disorder. An effective amount herein may vary according to factors such as the disease state, age, sex, and weight of the patient, and the ability of the antibody to elicit a desired response in the individual. An effective amount is also one in which any toxic or detrimental effects of the treatment are outweighed by the therapeutically beneficial effects. For prophylactic use, beneficial or desired results include results such as eliminating or reducing the risk, lessening the severity, or delaying the onset of the disease, including biochemical, histological and/or behavioral symptoms of the disease, its complications and intermediate pathological phenotypes presenting during development of the disease. For therapeutic use, beneficial or desired results include clinical results such as decreasing one or more symptoms resulting from the disease, increasing the quality of life of those suffering from the disease, decreasing the dose of other medications required to treat the disease, enhancing effect of another medication such as via targeting, delaying the progression of the disease, and/or prolonging survival. In the case of a cancer or tumour, an effective amount of the drug may have the effect in reducing the number of cancer cells; reducing the tumour size; inhibiting (i.e., slow to some extent or desirably stop) cancer cell infiltration into peripheral organs; inhibit (i.e., slow to some extent and desirably stop) tumour metastasis; inhibiting to some extent tumour growth; and/or relieving to some extent one or more of the symptoms associated with the cancer or tumour. An effective amount can be administered in one or more administrations. For purposes of this invention, an effective amount of drug, compound, or pharmaceutical composition is an amount sufficient to accomplish prophylactic or therapeutic treatment either directly or indirectly. As is understood in the clinical context, an effective amount of a drug, compound, or pharmaceutical composition may or may not be achieved in conjunction with another drug, compound, or pharmaceutical composition. Thus, an “effective amount” may be considered in the context of administering one or more therapeutic agents, and a single agent may be considered to be given in an effective amount if, in conjunction with one or more other agents, a desirable result may be or is achieved.

An “effective response” of a patient or a patient's “responsiveness” to treatment with a medicament and similar wording refers to the clinical or therapeutic benefit imparted to a patient at risk for, or suffering from, a disease or disorder, such as cancer. In one embodiment, such benefit includes any one or more of: extending survival (including overall survival and progression free survival); resulting in an objective response (including a complete response or a partial response); or improving signs or symptoms of cancer. A patient who “does not have an effective response” to treatment refers to a patient who does not have any one of extending survival (including overall survival and progression free survival); resulting in an objective response (including a complete response or a partial response); or improving signs or symptoms of cancer.

The term “elimination half-life” as used herein refers to the terminal log-linear rate of elimination of a peptide from the plasma of a subject. Those of skill in the art will appreciate that half-life is a derived parameter that changes as a function on both clearance and volume of distribution. The terms “extended”, “longer”, or “increased” used in the context of elimination half-life are used interchangeably herein and are intended to mean that there is a statistically significant increase in the half-life of a peptide (e.g., a bicyclic peptide) relative to that of the reference molecules (e.g., a linear L-amino acid peptide)as determined under comparable conditions.

The term “expression” refers the biosynthesis of a gene product. For example, in the case of a coding sequence, expression involves transcription of the coding sequence into mRNA and translation of mRNA into one or more polypeptides. Conversely, expression of a non-coding sequence involves transcription of the non-coding sequence into a transcript only. The term “expression” is also used herein to refer to the presence of a protein or molecule in a particular location and, thus, may be used interchangeably with “localization”.

The term “expression” with respect to a gene sequence refers to transcription of the gene to produce a RNA transcript (e.g., mRNA, antisense RNA, siRNA, shRNA, miRNA, etc.) and, as appropriate, translation of a resulting mRNA transcript to a protein. Thus, as will be clear from the context, expression of a coding sequence results from transcription and translation of the coding sequence. Conversely, expression of a non-coding sequence results from the transcription of the non-coding sequence.

The term “high”, as used herein, refers to a measure that is greater than normal, greater than a standard such as a predetermined measure or a subgroup measure or that is relatively greater than another subgroup measure. For example, CD44^{high} refers to a measure of CD44 that is greater than a normal CD44 measure. Consequently, “CD44^{hlflh}” always corresponds to, at the least, detectable CD44 in a relevant part of a subject's body or a relevant sample from a subject's body. A normal measure may be determined according to any method available to one skilled in the art. The term “high” may also refer to a measure that is equal to or greater than a predetermined measure, such as a predetermined cutoff. If a subject is not “high” for a particular marker, it is “low” for that marker. In general, the cut-off used for determining whether a subject is “high” or “low” should be selected such that the division becomes clinically relevant.

The term “host cell” includes an individual cell or cell culture which can be or has been a recipient of any recombinant vector(s) or isolated polynucleotide of the invention. Host cells include progeny of a single host cell and the progeny may not necessarily be completely identical (in morphology or in total DNA complement) to the original parent cell due to natural, accidental or deliberate mutation and/or change. A host cell includes cells transfected or infected in vivo or in vitro with a recombinant vector or a polynucleotide of the invention. A host cell which comprises a recombinant vector of the invention is a recombinant host cell.

“Hybridization” is used herein to denote the pairing of complementary nucleotide sequences to produce a DNA-DNA hybrid or a DNA-RNA hybrid. Complementary base sequences are those sequences that are related by the base-pairing rules. In DNA, A pairs with T and C pairs with G. In RNA U pairs with A and C pairs with G. In this regard, the terms “match” and “mismatch” as used herein refer to the hybridization potential of paired nucleotides in complementary nucleic acid strands. Matched nucleotides hybridize efficiently, such as the classical A-T and G-C base pair mentioned above. Mismatches are other combinations of nucleotides that do not hybridize efficiently. In the present invention, the preferred mechanism of pairing involves hydrogen bonding, which may be Watson-Crick, Hoogsteen or reversed Hoogsteen hydrogen bonding, between complementary nucleoside or nucleotide bases (nucleobases) of the strands of oligomeric compounds. For example, adenine and thymine are complementary nucleobases which pair through the formation of hydrogen bonds. Hybridization can occur under varying circumstances as known to those of skill in the art.

The term “inhibitor” as used herein refers to an agent that decreases or inhibits at least one function or biological activity of a target molecule.

As used herein, the term “isolated” refers to material that is substantially or essentially free from components that normally accompany it in its native state. For example, an “isolated peptide” refers to in vitro isolation and/or purification of a PD-L1 bicyclic peptide mimetic from its natural cellular environment and from association with other components of the cell. “Substantially free” means that a preparation of peptide is at least 10, 15, 20, 25, 30, 35, 40, 45, 50, 55, 60, 65, 70, 75, 80, 85, 90, 91, 92, 93, 94, 95, 96, 97, 98 or 99% pure. In a preferred embodiment, the preparation of peptide has less than about 30, 25, 20, 15, 10, 9, 8, 7 , 6, 5, 4, 3, 2 or 1% (by dry weight) of molecules that are not the subject of this invention (also referred to herein as “contaminating molecules”). When a peptide is recombinantly produced, it is also desirably substantially free of culture medium, i.e., culture medium represents less than about 20, 15, 10, 5, 4, 3, 2 or 1% of the volume of the preparation. The invention includes isolated or purified preparations of at least 0.01, 0.1, 1.0, and 10 milligrams in dry weight.

As used herein, “instructional material” includes a publication, a recording, a diagram, or any other medium of expression which can be used to communicate the usefulness of the compositions and methods of the invention. The instructional material of the kit of the invention may, for example, be affixed to a container which contains the therapeutic or diagnostic agents of the invention or be shipped together with a container which contains the therapeutic or diagnostic agents of the invention.

The term “liposome” refers to artificially prepared vesicles composed of lipid bilayers. Liposomes can be used for delivery of therapeutics due to their unique property of encapsulating a portion of an aqueous solution inside a lipophilic bilayer membrane. Lipophilic compounds can be dissolved in the lipid bilayer, and in this way liposomes can carry both lipophilic and hydrophilic compounds. To deliver the molecules to sites of action, the lipid bilayer can fuse with other bilayers such as cell membranes, thus delivering the liposome contents.

The term “mesenchymal phenotype” is understood in the art, and can be identified by morphological, molecular and/or functional characteristics. For example, mesenchymal cells generally have an elongated or spindle-shaped appearance, express the mesenchymal markers vimentin, fibronectin and N-cadherin, divide slowly or are non-dividing and/or have relatively high levels of motility, invasiveness and/or anchorage-independent growth as compared with epithelial cells.

As used herein, the term “mesenchymal-to-epithelial transition” (MET) is a reversible biological process that involves the transition from motile, multipolar or spindle-shaped mesenchymal cells to planar arrays of polarized cells called epithelia. MET is the reverse process of EMT. METs occur in normal development, cancer metastasis, and induced pluripotent stem cell reprogramming. In specific embodiments, MET refers to the reprogramming of cells that have undergone EMT to regain one or more epithelial characteristics (e.g., as described above). For example, such cells typically exhibit reduced motility and/or invasiveness and/or are rapidly dividing, and may thereby regain sensitivity to immunotherapeutics and/or cytotoxic agents.

As used herein, the terms “peptide”, “polypeptide” and “protein” are used interchangeably to refer to a polymer of amino acid residues and to variants and synthetic analogues of the same. Thus, these terms apply to amino acid polymers in which one or more amino acid residues is a synthetic non-naturally-occurring amino acid, such as a chemical analogue of a corresponding naturally-occurring amino acid, as well as to naturally-occurring amino acid polymers. These terms do not exclude modifications, for example, glycosylations, acetylations, phosphorylations and the like. Soluble forms of the subject peptides are particularly useful. Included within the definition are, for example, peptides containing one or more analogues of an amino acid including, for example, unnatural amino acids or polypeptides with substituted linkages.

The term “pharmaceutical composition” or “pharmaceutical formulation” refers to a preparation which is in such form as to permit the biological activity of the active ingredient(s) to be effective, and which contains no additional components which are unacceptably toxic to a subject to which the composition or formulation would be administered. Such formulations are sterile. “Pharmaceutically acceptable” excipients (vehicles, additives) are those which can reasonably be administered to a subject mammal to provide an effective dose of the active ingredient employed.

As used herein, the term “PD-L1 overexpressing cell” refers to a vertebrate cell, particularly a mammalian or avian (bird) cell, especially a mammalian cell, that expresses PD-L1 at a detectably greater level than a normal cell. The cell may be a vertebrate cell, such as a primate cell; an avian (bird) cell; a livestock animal cell (such as a sheep cell, cow cell, horse cell, deer cell, donkey cell and pig cell); a laboratory test animal cell (such as a rabbit cell, mouse cell, rat cell, guinea pig cell and hamster cell); a companion animal cell (such as a cat cell and dog cell); and a captive wild animal cell (such as a fox cell, deer cell and dingo cell). In particular embodiments, the PD-L1 overexpressing cell is a human cell. In specific embodiments, the PD-L1 overexpressing cell is a cancer stem cell or a non-cancer stem cell tumour cell; preferably a cancer stem cell tumour cell. Overexpression can be by 10%, 20%, 30%, 40%, 50%, 60%, 70%, 80%, 90% or more in comparison to a normal cell or comparison cell (e.g., a breast cell).

By “pharmaceutically acceptable carrier” is meant a pharmaceutical vehicle comprised of a material that is not biologically or otherwise undesirable, i.e., the material may be administered to a subject along with the selected active agent without causing any or a substantial adverse reaction. Carriers may include excipients and other additives such as diluents, detergents, coloring agents, wetting or emulsifying agents, pH buffering agents, preservatives, transfection agents and the like.

Similarly, a “pharmacologically acceptable” salt, ester, amide, prodrug or derivative of a compound as provided herein is a salt, ester, amide, prodrug or derivative that this not biologically or otherwise undesirable.

As used herein, the terms “prevent”, “prevented” or “preventing”, refer to a prophylactic treatment which increases the resistance of a subject to developing the disease or condition or, in other words, decreases the likelihood that the subject will develop the disease or condition as well as a treatment after the disease or condition has begun in order to reduce or eliminate it altogether or prevent it from becoming worse. These terms also include within their scope preventing the disease or condition from occurring in a subject which may be predisposed to the disease or condition but has not yet been diagnosed as having it.

By “radiation therapy” is meant the use of directed gamma rays or beta rays to induce sufficient damage to a cell so as to limit its ability to function normally or to destroy the cell altogether. It will be appreciated that there will be many ways known in the art to determine the dosage and duration of treatment. Typical treatments are given as a one-time administration and typical dosages range from 10 to 200 units (Grays) per day.

The terms “reduce”, “inhibit”, “suppress”, “decrease”, and grammatical equivalents when used in reference to the level of a substance and/or phenomenon in a first sample relative to a second sample, mean that the quantity of substance and/or phenomenon in the first sample is lower than in the second sample by any amount that is statistically significant using any art-accepted statistical method of analysis. In one embodiment, the reduction may be determined subjectively, for example when a patient refers to their subjective perception of disease symptoms, such as pain, fatigue, etc. In another embodiment, the reduction may be determined objectively, for example when the number of CSCs and/or non-CSC tumour cells in a sample from a patient is lower than in an earlier sample from the patient. In another embodiment, the quantity of substance and/or phenomenon in the first sample is at least 10% lower than the quantity of the same substance and/or phenomenon in a second sample. In another embodiment, the quantity of the substance and/or phenomenon in the first sample is at least 25% lower than the quantity of the same substance and/or phenomenon in a second sample. In yet another embodiment, the quantity of the substance and/or phenomenon in the first sample is at least 50% lower than the quantity of the same substance and/or phenomenon in a second sample. In a further embodiment, the quantity of the substance and/or phenomenon in the first sample is at least 75% lower than the quantity of the same substance and/or phenomenon in a second sample. In yet another embodiment, the quantity of the substance and/or phenomenon in the first sample is at least 90% lower than the quantity of the same substance and/or phenomenon in a second sample. Alternatively, a difference may be expressed as an “n-fold” difference.

As used herein, the terms “salts” and “prodrugs” include any pharmaceutically acceptable salt, ester, hydrate or any other compound which, upon administration to the recipient, is capable of providing (directly or indirectly) a PD-L1 bicyclic peptide mimetic of the invention, or an active metabolite or residue thereof. Suitable pharmaceutically acceptable salts include salts of pharmaceutically acceptable inorganic acids such as hydrochloric, sulfuric, phosphoric, nitric, carbonic, boric, sulfamic and hydrobromic acids, or salts of pharmaceutically acceptable organic acids such as acetic, propionic, butyric, tartaric, maleic, hydroxymaleic, fumaric, citric, lactic, mucic, gluconic, benzoic, succinic, oxalic, phenylacetic, methanesulfonic, toluenesulfonic, benzenesulfonic, salicylic, sulfanilic, aspartic, glutamic, edetic, stearic, palmitic, oleic, lauric, pantothenic, tannic, ascorbic and valeric acids. Base salts include, but are not limited to, those formed with pharmaceutically acceptable cations, such as sodium, potassium, lithium, calcium, magnesium, ammonium and alkylammonium. Also, basic nitrogen-containing groups may be quaternized with such agents as lower alkyl halides, such as methyl, ethyl, propyl and butyl chlorides, bromides and iodides; dialkyl sulfates such as dimethyl and diethyl sulfate; and others. However, it will be appreciated that non-pharmaceutically acceptable salts also fall within the scope of the invention since these may be useful in the preparation of pharmaceutically acceptable salts. The preparation of salts and prodrugs can be carried out by methods known in the art. For example, metal salts can be prepared by reaction of a compound of the invention with a metal hydroxide. An acid salt can be prepared by reacting an appropriate acid with a PD-L1 bicyclic peptide mimetic of the invention.

The term “sample” as used herein includes any biological specimen that may be extracted, untreated, treated, diluted or concentrated from a subject. Samples may include, without limitation, biological fluids such as whole blood, serum, red blood cells, white blood cells, plasma, saliva, urine, stool (i.e., feces), tears, sweat, sebum, nipple aspirate, ductal lavage, tumour exudates, synovial fluid, ascitic fluid, peritoneal fluid, amniotic fluid, cerebrospinal fluid, lymph, fine needle aspirate, amniotic fluid, any other bodily fluid, cell lysates, cellular secretion products, inflammation fluid, semen and vaginal secretions. Samples may include tissue samples and biopsies, tissue homogenates and the like. Advantageous samples may include ones comprising any one or more biomarkers as taught herein in detectable quantities. Suitably, the sample is readily obtainable by minimally invasive methods, allowing the removal or isolation of the sample from the subject. In certain embodiments, the sample contains blood, especially peripheral blood, or a fraction or extract thereof. Typically, the sample comprises blood cells such as mature, immature or developing leukocytes, including lymphocytes, polymorphonuclear leukocytes, neutrophils, monocytes, reticulocytes, basophils, coelomocytes, hemocytes, eosinophils, megakaryocytes, macrophages, dendritic cells natural killer cells, or fraction of such cells (e.g., a nucleic acid or protein fraction). In specific embodiments, the sample comprises leukocytes including peripheral blood mononuclear cells (PBMC).

The term “scaffold’ or “molecular scaffold” as used herein refers to a chemical moiety that is bonded to the peptide at the alkylamino linkages and thioether linkage (when cysteine is present) in the compositions of the invention. The term “scaffold molecule” or “molecular scaffold molecule” as used herein refers to a molecule that is capable of being reacted with a peptide or peptide ligand to form the derivatives of the invention having alkylamino and, in certain embodiments, also thioether bonds. Thus, the scaffold molecule has the same structure as the scaffold moiety except that respective reactive groups (such as leaving groups) of the molecule are replaced by alkylamino and thioether bonds to the peptide in the scaffold moiety.

The terms “subject”, “patient”, “host” or “individual” used interchangeably herein, refer to any subject, particularly a vertebrate subject, and even more particularly a mammalian subject, for whom therapy or prophylaxis is desired. Suitable vertebrate animals that fall within the scope of the invention include, but are not restricted to, any member of the subphylum Chordata including primates (e.g., humans, monkeys and apes, and includes species of monkeys such from the genus Macaca (e.g., cynomologus monkeys such as Macaca fascicularis, and/or rhesus monkeys (Macaca mulatta)) and baboon (Papio ursinus), as well as marmosets (species from the genus Callithrix), squirrel monkeys (species from the genus Saimiri) and tamarins (species from the genus Saguinus), as well as species of apes such as chimpanzees (Pan troglodytes)), rodents (e.g., mice rats, guinea pigs), lagomorphs (e.g., rabbits, hares), bovines (e.g., cattle), ovines (e.g., sheep), caprines (e.g., goats), porcines (e.g., pigs), equines (e.g., horses), canines (e.g., dogs), felines (e.g., cats), avians (e.g., chickens, turkeys, ducks, geese, companion birds such as canaries, budgerigars etc.), marine mammals (e.g., dolphins, whales), reptiles (snakes, frogs, lizards etc.), and fish. A preferred subject is a human in need of eliciting an immune response, including an immune response with enhanced T-cell activation. However, it will be understood that the aforementioned terms do not imply that symptoms are present.

As used herein, the terms “treatment”, “treating” and the like refer to clinical intervention designed to alter the natural course of the individual or cell being treated during the course of clinical pathology. Desirable effects of treatment include decreasing the rate of disease progression, ameliorating or palliating the disease state, and remission or improved prognosis. For example, an individual is successfully “treated” if one or more symptoms associated with a T-cell dysfunctional disorder are mitigated or eliminated, including, but are not limited to, reducing the proliferation of (or destroying) cancerous cells, reducing pathogen infection, decreasing symptoms resulting from the disease, increasing the quality of life of those suffering from the disease, decreasing the dose of other medications required to treat the disease, and/or prolonging survival of individuals.

As used herein, the term “tumour” refers to any neoplastic cell growth and proliferation, whether malignant or benign, and all pre-cancerous and cancerous cells and tissues. The terms “cancer” and “cancerous” refer to or describe the physiological condition in mammals that is typically characterized in part by unregulated cell growth. As used herein, the term “cancer” refers to non-metastatic and metastatic cancers, including early stage and late stage cancers. The term “precancerous” refers to a condition or a growth that typically precedes or develops into a cancer. The term “non-metastatic” refers to a cancer that is benign or that remains at the primary site and has not penetrated into the lymphatic or blood vessel system or to tissues other than the primary site. Generally, a non-metastatic cancer is any cancer that is a Stage 0, I or II cancer. By “early stage cancer” is meant a cancer that is not invasive or metastatic or is classified as a stage 0, I or II cancer. The term “late stage cancer” generally refers to a Stage III or IV cancer, but can also refer to a Stage II cancer or a substage of a Stage II cancer. One skilled in the art will appreciate that the classification of a Stage II cancer as either an early stage cancer or a late stage cancer depends on the particular type of cancer. Illustrative examples of cancer include, but are not limited to, breast cancer, prostate cancer, ovarian cancer, cervical cancer, pancreatic cancer, colorectal cancer, lung cancer, hepatocellular cancer, gastric cancer, liver cancer, bladder cancer, cancer of the urinary tract, thyroid cancer, renal cancer (kidney cancer), carcinoma, retinoblastoma, melanoma, brain cancer, non-small cell lung cancer, squamous cell cancer of the head and neck, endometrial cancer, multiple myeloma, mesothelioma, rectal cancer and esophageal cancer. In an exemplary embodiment, the cancer is breast cancer or melanoma.

Each embodiment described herein is to be applied mutatis mutandis to each and every embodiment unless specifically stated otherwise.

### 2. BICYCLIC PEPTIDES

The present invention is based in part of the determination that PD-L1 bicyclic peptide mimetics effectively stimulate the transition of a mesenchymal, resistant signature cancer cell to an epithelial responsive cancer cell. It was identified that this activity was due to the inhibition of or a reduction in the nuclear localization of PD-L1. In one or more embodiments, such bicyclic peptides inhibit or decrease the formation, maintenance, and/or viability of cancer stem cell and non-cancer stem cell tumour cells, and/or inhibit EMT and/or induce MET or cancer stem cell tumour cells. Thus, the inventors have conceived that the bicyclic peptides of the invention may be used for the treatment or prevention of cancer.

### 2.1 PD-L2 Bicyclic Peptide Mimetics

Accordingly, in one aspect of the invention, there is provided an isolated or purified PD-L1 bicyclic peptide mimetic comprising a polypeptide that comprises at least three cysteine residues, separated by at least two loop sequences, and a molecular scaffold which forms covalent bonds with the cysteine residues of the polypeptide such that at least two polypeptide loops are formed on the molecular scaffold, wherein the PD-L1 bicyclic peptide mimetic comprises an amino acid sequence of:

Z₁X₁C₁LX₂X₃IFC₂X₄LRKGX₅MX₆MDX₈KX₉

or a modified derivative, or pharmaceutically acceptable salt, thereof;
wherein:
C₁, C₂, and C₃ represent first, second and third cysteine residues, respectively;
X₁ is absent or alanine;
X₂ is selected from any small amino acid (optionally, threonine, glycine, serine, or alanine)
X₃ is selected from any amino acid;
X₄ is selected from any amino acid;
X₅ is selected from any amino acid;
X₆ is selected from any nonpolar/neutral amino acid (e.g., methionine, alanine, leucine, proline, glycine, isoleucine, phenylalanine, tryptophan, valine, and norleucine);
X₇ is selected from any nonpolar/neutral amino acid (e.g., valine, alanine, glycine, methionine, leucine, proline, isoleucine, phenylalanine, tryptophan, and norleucine);
X₈ is selected from any nonpolar/neutral amino acid (e.g., valine, alanine, methionine, glycine, leucine, proline, isoleucine, phenylalanine, tryptophan, and norleucine);
X₉ is selected from any amino acid; and
Z₁ is absent or is 1-tetradecanoic acid.

In some embodiments, the PD-L1 bicyclic peptide mimetic of Formula I comprises, consists or consists essentially of an amino acid sequence represented by SEQ ID NO: 1:

[SEQ ID NO: 1]
ACLTFIFCRLRKGRCMMDVKK.

In this regard, in some embodiments the PD-L1 bicycle peptide mimetic comprises, consists, or consists essentially of the following molecular structure:

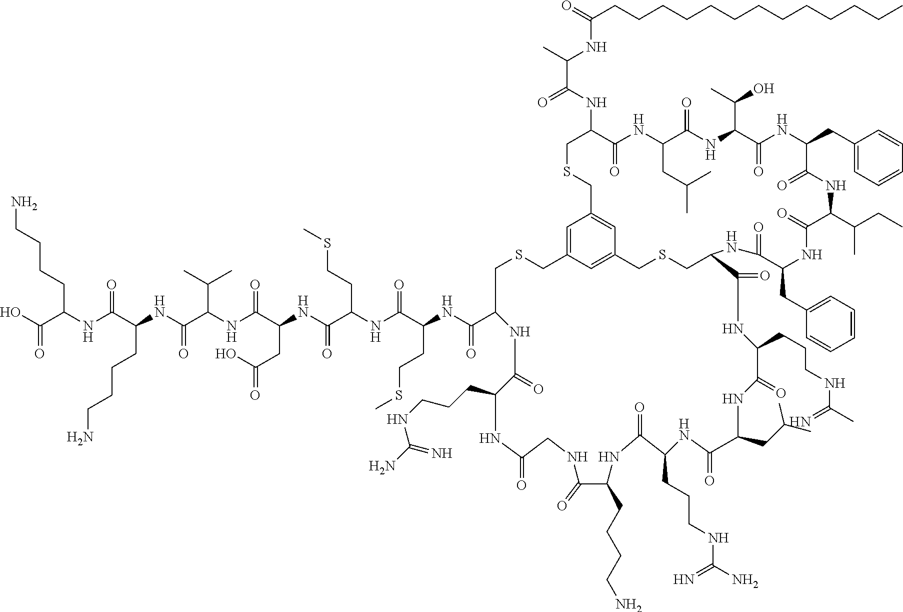 [SMILES: CCCCCCCCCCCCCC(=O)NC(C)C(=O)NC1CSCc2cc3cc(c2)CSC[C@H](NC(=O)[C@H](Cc2ccccc2)NC(=O)[C@H](C(C)CC)NC(=O)[C@H](Cc2ccccc2)NC(=O)[C@H]([C@@H](C)O)NC(=O)C(CC(C)C)NC1=O)C(=O)N[C@@H](CCCNC(C)=N)C(=O)N[C@@H](CC(C)C)C(=O)N[C@@H](CCCNC(=N)N)C(=O)N[C@@H](CCCCN)C(=O)NCC(=O)N[C@@H](CCCNC(=N)N)C(=O)NC(C(=O)N[C@@H](CCSC)C(=O)NC(CCSC)C(=O)N[C@@H](CC(=O)O)C(=O)NC(C(=O)N[C@@H](CCCCN)C(=O)NC(CCCCN)C(=O)O)C(C)C)CSC3]

In preferred embodiments, the PD-L1 bicyclic peptide mimetic of Formula I has any one or more activities selected from the group consisting of: (i) increasing cell death; (II) increasing MET; (ill) reducing or inhibiting EMT; (iv) inhibiting or reducing maintenance; (v) inhibiting or reducing proliferation; (vi) increasing differentiation; (vii) inhibiting or reducing formation; or (viii) reducing viability of a PD-L1-overexpressing cell. In some embodiments, the PD-L1-overexpressing cell is a cancer stem cell or a non-cancer stem cell tumour cell; especially a cancer stem cell tumour cell.

In some embodiments, the PD-L1 bicyclic peptide mimetic of Formula I has at least about 80%, 81%, 82%, 83%, 84%, 85%, 86%, 87%, 88%, 89%, 90%, 91%, 92%, 93%, 94%, 95%, 96%, 97%, 98%, or 99% sequence similarity to the amino acid sequence of SEQ ID NO: 1. In some embodiments, the PD-L1 bicyclic peptide mimetic of Formula I has at least about 80%, 81%, 82%, 83%, 84%, 85%, 86%, 87%, 88%, 89%, 90%, 91%, 92%, 93%, 94%, 95%, 96%, 97%, 98%, or 99% sequence identity to the amino acid sequence of SEQ ID NO: 1.

The present invention also contemplates PD-L1 bicyclic peptide mimetics that are variants of SEQ ID NO: 1. Such “variant” PD-L1 bicyclic peptide mimetics include PD-L1 bicyclic peptide mimetics derived from SEQ ID NO: 1 by deletion or addition of one or more amino acids to the N-terminal and/or C-terminal end of the PD-L1 bicyclic peptide mimetic, deletion or addition of one or more amino acids at one or more sites in the PD-L1 bicyclic peptide mimetic, or substitution of one or more amino acids at one or more sites in the PD-L1 bicyclic peptide mimetic.

Variant PD-L1 bicyclic peptide mimetics encompassed by the present invention are biologically active, that is, they continue to possess the desired biological activity of the native PD-L1 bicyclic peptide mimetic.

The PD-L1 bicyclic peptide mimetics of SEQ ID NO: 1 may be altered in various ways, including amino acid substitutions, deletions, truncations and insertions. Methods for such manipulations are generally known in the art. For example, amino acid sequence variants of SEQ ID NO: 1 may be prepared by mutagenesis of nucleic acids encoding the amino acid sequence of any one of SEQ ID NO: 1. Methods for mutagenesis and nucleotide sequence alterations are well known in the art. Refer to, for example, Kunkel (1985, Proc. Natl. Acad. Sd. USA. 82: 488-492), Kunkel et al., (1987, Methods In Enzymol, 154: 367-382), U.S. Pat. No. 4,873,192, Watson, J. D. et al., (“Molecular Biology of the Gene”, Fourth Edition, Benjamir V. Cummings, Menlo Park, Calif., 1987) and the references cited therein. Guidance as to appropriate amino acid substitutions that do not affect biological activity of the PD-L1 bicyclic peptide mimetics of interest may be found in the model of Dayhoff et al., (1978) Atlas of Protein Sequence and Structure (Natl. Biomed. Res. Found., Washington, D.C.). Methods for screening gene products of combinatorial libraries made by point mutations or truncation, and for screening cDNA libraries for gene products having a selected property are known in the art. Such methods are adaptable for rapid screening of the gene libraries generated by combinatorial mutagenesis of the PD-L1 bicyclic peptide mimetic of SEQ ID NO: 1. Recursive ensemble mutagenesis (REM), a technique which enhances the frequency of functional mutants in the libraries, can be used in combination with screening assays to identify active variants (Arkin and Yourvan (1992) Pnoc. Natl. Acad. Sd. USA 89: 7811-7815; Delgrave et al., (1993) Protein Engineering, 6: 327-331). Conservative substitutions, such as exchanging one amino acid with another having similar properties, may be desirable as discussed in more detail below.

Variant PD-L1 bicyclic peptide mimetics of the invention may contain conservative amino acid substitutions at various locations along their sequence, as compared to a parent (e.g., reference) amino acid sequence, such as SEQ ID NO: 1. A “conservative amino acid substitution” is one in which the amino acid residue is replaced with an amino acid residue having a similar side chain. Families of amino acid residues having similar side chains have been defined in the art as discussed in detail below.

Acidic: The residue has a negative charge due to loss of a proton at physiological pH and the residue is attracted by aqueous solution so as to seek the surface positions in the conformation of a peptide in which it is contained when the peptide is in aqueous medium at physiological pH. Amino acids having an acidic side chain include glutamic acid and aspartic acid.

Basic: The residue has a positive charge due to association with protons at physiological pH or within one or two pH units thereof (e.g., histidine) and the residue is attracted by aqueous solution so as to seek the surface positions in the conformation of a peptide in which it is contained when the peptide is in aqueous medium at physiological pH. Amino acids having a basic side chain include arginine, lysine and histidine.

Charged: The residue is charged at physiological pH and, therefore, includes amino acids having acidic or basic side chains, such as glutamic acid, aspartic acid, arginine, lysine and histidine.

Hydrophobic: The residue is not charged at physiological pH and the residue is repelled by aqueous solution so as to seek the inner positions in the conformation of a peptide in which it is contained when the peptide is in aqueous medium at physiological pH. Amino acids having a hydrophobic side chain include tyrosine, valine, isoleucine, leucine, methionine, norleucine, phenylalanine and tryptophan.

Neutral/polar: The residues are not charged at physiological pH but the residue is not sufficiently repelled by aqueous solutions so that it would seek inner positions in the conformation of a peptide in which it is contained when the peptide is in aqueous medium at physiological pH. Amino acids having a neutral/polar side chain include asparagine, glutamine, cysteine, histidine, serine and threonine.

This description also characterizes certain amino acids as “small”” since their side chains are not sufficiently large, even if polar groups are lacking, to confer hydrophobicity. With the exception of proline, “small” amino acids are those with four carbons or less when at least one polar group is on the side chain and three carbons or less when not. Amino acids having a small side chain include glycine, serine, alanine and threonine. The gene-encoded secondary amino acid proline is a special case due to its known effects on the secondary conformation of peptide chains. The structure of proline differs from all the other naturally-occurring amino acids in that its side chain is bonded to the nitrogen of the α-amino group, as well as the α-carbon. Several amino acid similarity matrices (e.g., PAM120 matrix and PAM250 matrix as disclosed for example by Dayhoff et al., (1978), A model of evolutionary change in proteins. Matrices for determining distance relationships In M. Dayhoff, (ed.), Atlas of protein sequence and structure, Vol. 5, pp. 345-358, National Biomedical Research Foundation, Washington DC; and by Gonnet et al., (1992), Science, 256(5062): 1443-1445), however, include proline in the same group as glycine, serine, alanine and threonine. Accordingly, for the purposes of the present invention, proline is classified as a “small” amino acid.

The degree of attraction or repulsion required for classification as polar or non-polar is arbitrary and, therefore, amino acids specifically contemplated by the invention have been classified as one or the other. Most amino acids not specifically named can be classified on the basis of known behavior.

Amino acid residues can be further sub-classified as cyclic or non-cyclic, and aromatic or non-aromatic, self-explanatory classifications with respect to the side-chain substituent groups of the residues, and as small or large. The residue is considered small if it contains a total of four carbon atoms or less, inclusive of the carboxyl carbon, provided an additional polar substituent is present; three or less if not. Small amino acid residues are, of course, always non-aromatic. Dependent on their structural properties, amino acid residues may fall in two or more classes. For the naturally-occurring protein amino acids, sub-classification according to this scheme is presented in Table 1.

TABLE 1
Amino Acid Sub-Classification

| SUB-CLASSES | AMINO ACIDS |
|---|---|
| Acidic | Aspartic acid, Glutamic acid |
| Basic | Noncyclic: Arginine, lysine; |
| | Cyclic: histidine |
| Charged | Aspartic acid, glutamic acid, arginine, lysine, |
| | histidine |
| Small | Glycine, serine, alanine, threonine, proline |
| Nonpolar/neutral | Alanine, glycine, isoleucine, leucine, methionine, |
| | phenylalanine, proline, tryptophan, valine |
| Polar/neutral | Asparagine, histidine, glutamine, cysteine, serine, |
| | threonine, tyrosine |
| Polar/negative | Aspartic acid, glutamic acid |
| Polar/positive | Lysine, arginine |
| Polar/large | Asparagine, glutamine |
| Polar | Arginine, asparagine, aspartic acid, cysteine, |
| | glutamic acid, glutamine, histidine, lysine, serine, |
| | threonine, tyrosine |
| Hydrophobic | Tyrosine, valine, isoleucine, leucine, methionine, |
| | phenylalanine, tryptophan |
| Aromatic | Tryptophan, tyrosine, phenylalanine |
| Residues that influence | Glycine and proline |
| chain orientation | |

Conservative amino acid substitution also includes groupings based on side chains. For example, a group of amino acids having aliphatic side chains is glycine, alanine, valine, leucine, isoleucine and norleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. For example, it is reasonable to expect that replacement of a leucine with an isoleucine or valine, an aspartic acid with a glutamic acid, a threonine with a serine, or a similar replacement of an amino acid with a structurally related amino acid will not have a major effect on the properties of the resulting variant peptide of the invention. Whether an amino acid change results in a PD-L1 bicyclic peptide mimetic that inhibits or reduces the nuclear localization of a nuclear localizable polypeptide, such as PD-1, PD-L1 and/or PD-L2, can readily be determined by assaying its activity. Conservative substitutions are shown in Table 2 under the heading of exemplary and preferred substitutions. Amino acid substitutions falling within the scope of the invention, are, in general, accomplished by selecting substitutions that do not differ significantly in their effect on maintaining (a) the structure of the peptide backbone in the area of the substitution, (b) the charge or hydrophobicity of the molecule at the target site, or (c) the bulk of the side chain. After the substitutions are introduced, the variants are screened for biological activity.

TABLE 2
EXEMPLARY AND PREFERRED
AMINO ACID SUBSTITUTIONS

| ORIGINAL | | PREFERRED |
|---|---|---|
| RESIDUE | EXEMPLARY SUBSTITUTIONS | SUBSTITUTION |
| Ala | Val, Leu, Ile | Val |
| Arg | Lys, Gln, Asn | Lys |
| Asn | Gln, His, Lys, Arg | Gln |
| Asp | Glu | Glu |
| Cys | Ser | See |
| Gln | Asn, His, Lys | Asn |
| Glu | Asp, Lys | Asp |
| Gly | Pro | Pro |
| His | Asn, Gln, Lys, Arg | Arg |
| Ile | Leu, Val, Met, Ala, Phe, Norleu | Leu |
| Leu | Norleu, Ile, Val, Met, Ala, Phe | Ile |
| Lys | Arg, Gln, Asn | Arg |
| Met | Leu, Ile, Phe | Leu |
| Phe | Leu, Vel, Ile, Ala | Leu |
| Pro | Gly | Gly |
| Ser | Thr | Thr |
| Thr | Ser | Ser |
| Trp | Tyr | Tyr |

Alternatively, similar amino acids for making conservative substitutions can be grouped into three categories based on the identity of the side chains. The first group includes glutamic acid, aspartic acid, arginine, lysine and histidine, which all have charged side chains; the second group includes glycine, serine, threonine, cysteine, tyrosine, glutamine and asparagine; and the third group includes leucine, isoleucine, valine, alanine, proline, phenylalanine, tryptophan, methionine and norleucine, as described in Zubay, Biochemistry, third edition, Wm.C. Brown Publishers (1993).

Thusn-essential amino acid residue in a PD-L1 bicyclic peptide mimetic of the invention is typically replaced with another amino acid residue from the same side chain family. Alternatively, mutations can be introduced randomly along all or part of the coding sequence of a PD-L1 bicyclic peptide mimetic of the invention, such as by saturation mutagenesis, and the resultant mutants can be screened for an activity of the parent polypeptide, as described for example herein, to identify mutants which retain that activity. Following mutagenesis of the coding sequences, the encoded PD-L1 bicyclic peptide mimetic can be expressed recombinantly and its activity determined. A “non-essential” amino acid residue is a residue that can be altered from the reference sequence of an embodiment PD-L1 bicyclic peptide mimetic of the invention without abolishing or substantially altering one or more of its activities. Suitably, the alteration does not substantially alter one of these activities, for example, the activity is at least 20%, 40%, 60%, 70% or 80% of that of the wild-type. By contrast, an “essential” amino acid residue is a residue that, when altered from the wild-type sequence of an embodiment PD-L1 bicyclic peptide mimetic of the invention, results in abolition of an activity of the parent molecule such that less than 20% of the wild-type activity is present.

Accordingly, the present invention also contemplates variants of the PD-L1 bicyclic peptide mimetic of SEQ ID NO: 1, wherein the variants are distinguished from the parent sequence by the addition, deletion, or substitution of one or more amino acid residues. In general, variants will display at least about 80%, 81%, 82%, 83%, 84%, 85%, 86%, 87%, 88%, 89%, 90%, 91%, 92%, 93%, 94%, 95%, 96%, 97%, 98%, 99% sequence similarity to a reference PD-L1 bicyclic peptide mimetic sequence as, for example, set forth in SEQ ID NO: 1, as determined by sequence alignment programs described elsewhere herein using default parameters. Desirably, variants will have at least 80%, 81%, 82%, 83%, 84%, 85%, 86%, 87%, 88%, 89%, 90%, 91%, 92%, 93%, 94%, 95%, 96%, 97%, 98%, 99% sequence identity to a parent or reference PD-L1 bicyclic peptide mimetic sequence as, for example, set forth in SEQ ID NO: 1, as determined by sequence alignment programs described herein using default parameters. Variants of SEQ ID NO: 1 which fall within the scope of a variant PD-L1 bicyclic peptide mimetic of the invention, may differ from the parent molecule generally by at least 1, but by less than 5, 4, 3, 2 or 1 amino acid residue(s). In some embodiments, a variant PD-L1 bicyclic peptide mimetic of the invention differs from the corresponding sequence in SEQ ID NO: 1 by at least 1, but by less than 5, 4, 3, 2 or 1 amino acid residue(s). In some embodiments, the amino acid sequence of the variant PD-L1 bicyclic peptide mimetic of the invention comprises the PD-L1 bicyclic peptide mimetic of Formula I. In particular embodiments, the variant PD-L1 bicyclic peptide mimetic of the invention inhibits or reduces nuclear localization of PD-L1.

If the sequence comparison requires alignment, the sequences are typically aligned for maximum similarity or identity. “Looped” out sequences from deletions or insertions, or mismatches, are generally considered differences. The differences are, suitably, differences or changes at a non-essential residue or a conservative substitution.

In some embodiments, calculations of sequence similarity or sequence identity between sequences are performed as follows:

To determine the percent identity of two amino acid sequences or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In some embodiments, the length of a reference sequence aligned for comparison purposes is at least 40%, more usually at least 50% or 60%, and even more usually at least 70%, 80%, 90% or 100% of the length of the reference sequence. The amino acid residues or nucleotides at corresponding amino acid positions or nucleotide positions are then compared. When a position in the first sequence is occupied by the same amino acid residue or nucleotide at the corresponding position in the second sequence, then the molecules are identical at that position. For amino acid sequence comparison, when a position in the first sequence is occupied by the same or similar amino acid residue (i.e., conservative substitution) at the corresponding position in the second sequence, then the molecules are similar at that position.

The percent identity between the two sequences is a function of the number of identical amino acid residues shared by the sequences at individual positions, taking into account the number of gaps and the length of each gap, which need to be introduced for optimal alignment of the two sequences. By contrast, the percent similarity between the two sequences is a function of the number of identical and similar amino acid residues shared by the sequences at individual positions, taking into account the number of gaps and the length of each gap, which need to be introduced for optimal alignment of the two sequences.

The comparison of sequences and determination of percent identity or percent similarity between sequences can be accomplished using a mathematical algorithm. In certain embodiments, the percent identity or similarity between amino acid sequences is determined using the Needleman and WQnsch, (1970, J. Mol. Biol., 48: 444-453) algorithm which has been incorporated into the GAP program in the GCG software package (Devereaux, et al. (1984) Nucleic Adds Research, 12: 387-395), using either a Blosum 62 matrix or a PAM250 matrix, and a gap weight of 16, 14, 12, 10, 8, 6, or 4 and a length weight of 1, 2, 3, 4, 5, or 6. In some embodiments, the percent identity or similarity between amino acid sequences can be determined using the algorithm of Meyers and Miller (1989, Cablos, 4: 11-17) which has been incorporated into the ALIGN program (version 2.0), using a PAM120 weight residue table, a gap length penalty of 12 and a gap penalty of 4.

The present invention also contemplates an isolated or purified PD-L1 bicyclic peptide mimetic that is encoded by a polynucleotide sequence that hybridizes under stringency conditions as defined herein, especially under medium, high or very high stringency conditions, preferably under high or very high stringency conditions, to a polynucleotide sequence encoding the PD-L1 bicyclic peptide mimetic of SEQ ID NO: 1 or the non-coding strand thereof. The invention also contemplates an isolated nucleic acid molecule comprising a polynucleotide sequence that hybridizes under stringency conditions as defined herein, especially under medium, high or very high stringency conditions, preferably under high or very high stringency conditions, to a polynucleotide sequence encoding the PD-L1 bicyclic peptide mimetic of SEQ ID NO: 1, or the non-coding strand thereof.

As used herein, the term “hybridizes under stringency conditions” describes conditions for hybridization and washing and may encompass low stringency, medium stringency, high stringency and very high stringency conditions.

Guidance for performing hybridization reactions can be found in Ausubel, et al. (1998) Current Protocols in Molecular Biology (John Wiley and Sons, Inc.), in particular sections 6.3.1-6.3.6. Both aqueous and non-aqueous methods can be used. Reference herein to low stringency conditions include and encompass from at least about 1% v/v to at least about 15% v/v formamide and from at least about 1 M to at least about 2 M salt for hybridization at 42° C., and at least about 1 M to at least about 2 M salt for washing at 42° C. Low stringency conditions also may include 1% bovine serum albumin (BSA), 1 mM EDTA, 0.5 M NaHPO₄ (pH 7.2), 7% sodium dodecyl sulfate (SDS) for hybridization at 65° C., and (i) 2^{c} sodium chloride/sodium citrate (SSC), 0.1% SDS; or (II) 0.5% BSA, 1 mM EDTA, 40 mM NaHPO₄ (pH 7.2), 5% SDS for washing at room temperature. One embodiment of low stringency conditions includes hybridization in 6^{c} SSC at about 45° C., followed by two washes in 0.2× SSC, 0.1% SDS at least at 50 C (the temperature of the washes can be increased to 55° C. for low stringency conditions). Medium stringency conditions include and encompass from at least about 16% v/v to at least about 30% v/v formamide and from at least about 0.5 M to at least about 0.9 M salt for hybridization at 42° C., and at least about 0.1 M to at least about 0.2 M salt for washing at 55° C. Medium stringency conditions also may include 1% bovine serum albumin (BSA), 1 mM EDTA, 0.5 M NaHPO₄ (pH 7.2), 7% SDS for hybridization at 65° C., and (i) 2× SSC, 0.1% SDS; or (II) 0.5% BSA, 1 mM EDTA, 40 mM NaHPO₄ (pH 7.2), 5% SDS for washing at 60-65° C. One embodiment of medium stringency conditions includes hybridizing in 6× SSC at about 45° C., followed by one or more washes in 0.2× SSC, 0.1% SDS at 60° C. High stringency conditions include and encompass from at least about 31% v/v to at least about 50% v/v formamide and from about 0.01 M to about 0.15 M salt for hybridization at 42° C., and about 0.01 M to about 0.02 M salt for washing at 55° C. High stringency conditions also may include 1% BSA, 1 mM EDTA, 0.5 M NaHPO₄ (pH 7.2), 7% SDS for hybridization at 65° C., and (i) 0.2× SSC, 0.1% SDS; or (II) 0.5% BSA, 1 mM EDTA, 40 mM NaHPO₄ (pH 7.2), 1% SDS for washing at a temperature in excess of 65° C. One embodiment of high stringency conditions includes hybridizing in 6× SSC at about 45° C., followed by one or more washes in 0.2× SSC, 0.1% SDS at 65° C.

In some aspects of the present invention, there is provided an isolated or purified PD-L1 bicyclic peptide mimetic of the invention that is encoded by a polynucleotide sequence that hybridizes under high stringency conditions to a polynucleotide sequence encoding the PD-L1 bicyclic peptide mimetic of SEQ ID NO: 1, or the non-coding strand thereof. In certain embodiments, the isolated or purified PD-L1 bicyclic peptide mimetic of the invention is encoded by a polynucleotide sequence that hybridizes under very high stringency conditions to a polynucleotide sequence encoding the PD-L1 bicyclic peptide mimetic of SEQ ID NO: 1, or the non-coding strand thereof. One embodiment of very high stringency conditions includes hybridizing 0.5 M sodium phosphate, 7% SDS at 65° C., followed by one or more washes at 0.2× SSC, 1% SDS at 65° C. In some embodiments, the amino acid sequence of the variant PD-L1 bicyclic peptide mimetic of the invention comprises the amino acid sequence of Formula I. In particular embodiments, the variant PD-L1 bicyclic peptide mimetic of the invention inhibits or reduces nuclear localization PD-L1.

Other stringency conditions are well known in the art and a person skilled in the art will recognize that various factors can be manipulated to optimize the specificity of the hybridization. Optimization of the stringency of the final washes can serve to ensure a high degree of hybridization. For detailed examples, see Ausubel, et al. (1998) Current Protocols in Molecular Biology (John Wiley and Sons, Inc.), in particular pages 2.10.1 to 2.10.16 and Sambrook, et al. (1989) Molecular Cloning: A Laboratory Manual (Cold Spring Harbour Press), in particular Sections 1.101 to 1.104.

While stringent washes are typically carried out at temperatures from about 42° C. to 68° C., a person skilled in the art will appreciate that other temperatures may be suitable for stringent conditions. Maximum hybridization rate typically occurs at about 20° C. to 25° C. below the Tr, for formation of a DNA-DNA hybrid. It is well known in the art that the Tₘ is the melting temperature, or temperature at which two complementary polynucleotide sequences dissociate. Methods for estimating Tₘ are well known in the art (see Ausubel, et al. (1998) Current Protocols in Molecular Biology (John Wiley and Sons, Inc.) at page 2.10.8). In general, the Tₘ of a perfectly matched duplex of DNA may be predicted as an approximation by the formula:

Tₘ=81.5+16.6 (log₁₀ M)+0.41 (% G+C)−0.63 (% formamide)−(600/length)
wherein: M is the concentration of Na⁺, preferably in the range of 0.01 M to 0.4 M; % G+C is the sum of guanosine and cytosine bases as a percentage of the total number of bases, within the range between 30% and 75% G+C; % formamide is the percent formamide concentration by volume; length is the number of base pairs in the DNA duplex. The Tₘ of a duplex DNA decreases by approximately 1° C. with every increase of 1% in the number of randomly mismatched base pairs. Washing is generally carried out at Tₘ−15° C. for high stringency, or Tₘ−30° C. for moderate stringency.

In one example of a hybridization procedure, a membrane (e.g., a nitrocellulose membrane or a nylon membrane) containing immobilized DNA is hybridized overnight at 42° C. in a hybridization buffer (50% deionized formamide, 5 c SSC, 5 c Denhardt's solution (0.1% ficoll, 0.1% polyvinylpyrrolidone and 0.1% BSA), 0.1% SDS and 200 mg/mL denatured salmon sperm DNA) containing labeled probe. The membrane is then subjected to two sequential medium stringency washes (i.e., 2× SSC, 0.1% SDS for 15 min at 45° C., followed by 2× SSC, 0.1% SDS for 15 min at 50° C.), followed by two sequential higher stringency washes (i.e., 0.2× SSC, 0.1% SDS for 12 min at 55° C. followed by 0.2× SSC and 0.1% SDS solution for 12 min at 65-68° C.

The PD-L1 bicyclic peptide mimetics of the present invention also encompass a PD-L1 bicyclic peptide mimetic comprising amino acids with modified side chains, incorporation of unnatural amino acid residues and/or their derivatives during peptide synthesis and the use of cross-linkers and other methods which impose conformational constraints on the PD-L1 bicyclic peptide mimetics of the invention. Examples of side chain modifications include modifications of amino groups, such as by acylation with acetic anhydride; acylation of amino groups with succinic anhydride and tetrahydrophthalic anhydride; amidination with methylacetimidate; carbamoylation of amino groups with cyanate; pyridoxylation of lysine with pyridoxal-5-phosphate followed by reduction with sodium borohydride; reductive alkylation by reaction with an aldehyde followed by reduction with sodium borohydride; and trinitrobenzylation of amino groups with 2,4,6-tri nitrobenzene sulfonic acid (TNBS).

The carboxyl group may be modified by carbodiimide activation through O-acylisourea formation followed by subsequent derivatization, for example, to a corresponding amide.

The guanidine group of arginine residues may be modified by formation of heterocyclic condensation products with reagents such as 2,3-butanedione, phenylglyoxal and glyoxal.

Examples of incorporating unnatural amino acids and derivatives during peptide synthesis include, but are not limited to, use of 4-amino butyric acid, 6-aminohexanoic acid, 4-amino-3-hydroxy-5-phenylpentanoic acid, 4-amino-3-hydroxy-6-methylheptanoic acid, t-butylglycine, norleucine, norvaline, phenylglycine, ornithine, /Vₑ-acetyl-L-ornithine, sarcosine, 2-thienyl alanine, L/e-acetyl-L-lysine, L/e-methyl-L-lysine, L/e-dimethyl-L-lysine, L/e-formyl-L-lysine and/or D-isomers of amino acids. A list of unnatural amino acids contemplated by the present invention is shown in Table 3.

TABLE 3
NON-CONVENTIONAL AMINO ACIDS

| | |
|---|---|
| α-aminobutyric acid | L-N-methylalanine |
| α-amino-α-methylbutyrate | L-N-methylarginine |
| Aminocyclopropane-carboxylate | L-N-methylasparagine |
| Aminoisobutyric acid | L-N-methylaspartic acid |
| Aminonorbornyl-carboxylate | L-N-methylcysteine |
| Cyclohexylalanine | L-N-methylglutamine |
| Ayclopentylalanine | L-N-methylglutamic acid |
| L-N-methylisoleucine | L-N-methylhistidine |
| D-alanine | L-N-methylleucine |
| D-arginine | L-N-methyllysine |
| D-aspartic acid | L-N-methylmethionine |
| D-cysteine | L-N-methylnorleucine |
| D-glutamate | L-N-methylnorvaline |
| D-glutamic acid | L-N-methylornithine |
| D-histidine | L-N-methylphenylalanine |
| D-isoleucine | L-N-methylproline |
| D-leucine | L-N-methylserine |
| D-lysine | L-N-methylthreonine |
| D-methionine | L-N-methyltryptophan |
| D-ornithine | L-N-methyltyrosine |
| D-phenylalanine | L-N-methylvaline |
| D-proline | L-N-methylethylglycine |
| D-serine | L-N-methyl-t-butylglycine |
| D-threonine | L-norleucine |
| D-tryptophan | L-norvaline |
| D-tyrosine | α-methyl-aminoisobutyrate |
| D-valine | α-γ-aminobutyrate |
| D-α-methylalanine | α-methylcyclohexylalanine |
| D-α-methylasparagine | D-α-methyl-α-napthylalanine |
| D-α-methylaspartate | D-α-methylpenicillamine |
| D-α-methylcysteine | N-(4-aminobutyl)glycine |
| D-α-methylhistidine | N-(2-aminoethyl)glycine |
| D-α-methylisoleucine | N-(3-aminopropyl)glycine |
| D-α-methylleucine | N-aminog-methylbutyrate |
| D-α-methyllysine | α-napthylalanine |
| D-α-methylmethionine | N-benzylglycine |
| D-α-methylornithine | N-(2-carbamylediyl)glycine |
| D-α-methylpheylalanine | N-(caramylmethyl)glycine |
| D-α-methylproline | N-(2-carboxyethyl)glycine |
| D-α-methylserine | N-(carboxymethyl)glycine |
| D-α-methylthreonine | N-cyclobutylglycine |
| D-α-methyltryptophan | N-cycloheptylglycine |
| D-α-methyltyrosine | N-cyclodecylglycine |
| L-α-methylleucine | L-α-methyllysine |
| L-α-methylmethionine | L-α-methylnorleucine |
| L-α-methylnorvatine | L-α-methylornithine |
| L-α-methylphenylalanine | L-α-methylproline |
| L-α-methylserine | L-α-methylthreonine |
| L-α-methyltryptophan | L-α-methyltyrosine |
| L-α-methylvaline | L-N-methylhomophenylalanine |
| N-(N-2,2-diphenylethyl | N-(N-(3,3-diphenylpropyl |
| carbamylmethyl)glycine | carbnamylmethyl)glycine |
| 1-carboxy-1-1(2,2-diphenyl-ethyl | |
| amino)cyclopropane | |

In some embodiments, the PD-L1 bicyclic peptide mimetics of the invention comprises at least one unnatural amino acid.

### 2.2 D-Amino Acid Peptides

In some embodiments, at least one amino acid of the PD-L1 bicyclic peptide mimetics described above and/or elsewhere herein is a D-amino acid. Preferably, the D-amino acid corresponds to the native L-amino acid. An advantage of embodiments of this type is that peptides incorporating at least one D-amino acid generally have a higher stability, longer bioavailability, and/or slower elimination half-life than peptides comprised solely of L-amino acids.

In some embodiments, the PDL1 bicyclic peptide mimetics of the invention are comprised solely of D-amino acid residues (except for glycine, which does not have a stereoisomer). In other embodiments, at least 5%, 10%, 15%, 20%, 25%, 30%, 35%, 40%, 45%, 50%, 55%, 60%, 65%, 70%, 75%, 80%, 85%, 90%, or 95% of the amino acids of the PD-L1 bicyclic peptide mimetic are D-amino acids.

In some embodiments, residues corresponding to residues known or predicted to proteolytic cleavage sites and/or otherwise susceptible to degradation are substituted with a corresponding D-amino acid isoform. By way of an illustrative example, the PD-L1 bicyclic peptide mimetic may comprise an amino acid sequence that corresponds to the NLS motif of PD-L1, and any one of the key NLS residues corresponding to leucine at position 261 of the wild-type PD-L1 amino acid sequence (as set forth in SEQ ID NO: 75), arginine at position 262 of the wild type PD-L1 amino acid sequence (as set forth in SEQ ID NO: 75), and/or lysine at position 263 of the wild-type PD-L1 amino acid sequence (as set forth in SEQ ID NO: 75), are substituted with corresponding D-amino acids.

In some embodiments, the PD-L1 bicyclic peptide mimetic of Formula I comprises a D-leucine at the position corresponding to position 261 of the native PD-L1 sequence set forth in SEQ ID NO: 75. In some of the same embodiments and some alternative embodiments, the PD-L1 bicyclic peptide mimetic of Formula I comprises a D-arginine at the position corresponding to position 262 of the native PD-L1 sequence set forth in SEQ ID NO: 75. At some of the same embodiments and some alternative embodiments, the PD-L1 bicyclic peptide mimetic of Formula I comprises a D-lysine at the position corresponding to position 263 of the native PD-L1 amino acid sequence set forth in SEQ ID NO: 75. In some embodiments, the PD-L1 bicyclic peptide mimetic comprises two of a D-leucine at the position corresponding to position 261 of the native PD-L1 sequence set forth in SEQ ID NO: 75, a D-arginine at the position corresponding to position 262 of the native PD-L1 sequence set forth in SEQ ID NO: 75, and a D-lysine at the position corresponding to position 263 of the native PD-L1 amino acid sequence set forth in SEQ ID NO: 75. In some embodiments, the PD-L1 bicyclic peptide mimetic comprises a D-leucine at the position corresponding to position 261 of the native PD-L1 sequence set forth in SEQ ID NO: 75, a D-arginine at the position corresponding to position 262 of the native PD-L1 sequence set forth in SEQ ID NO: 75, and a D-lysine at the position corresponding to position 263 of the native PD-L1 amino acid sequence set forth in SEQ ID NO: 75.

In some of the same embodiments and some other embodiments, the PD-L1 bicyclic peptide mimetic of Formula (I) may comprise a chain of D-amino acids in a retro-inverso configuration. In embodiments of this type, the entire peptide may be in retro-inverso form, or a portion of the peptide may be in retro-inverso form. By way of an example, in the PD-L1 bicyclic peptide mimetics of Formula (I), the “LRK” residues (i.e., corresponding to residues 261-263 of the native PD-L1 amino acid sequence set forth in SEQ ID NO: 75) may be D-amino acids, and in retro-inverso form. In such embodiments, these residues are in the format (i.e., D-Lys-D-Arg-D-Leu).

### 2.3 Terminal Peptide Modifications

Additional amino acids or other substituents may be added to the N- or C-termini, if present, of the PD-L1 bicyclic peptide mimetics of the invention. For example, the PD-L1 bicyclic peptide mimetics of the invention may form part of a longer sequence with additional amino acids added to either or both of the N- and C-termini.

For particular uses and methods of the invention, PD-L1 bicyclic peptide mimetics with high levels of stability may be desired, for example, to increase the elimination half-life of the PD-L1 bicyclic peptide mimetic in a subject. Thus, in some embodiments, the PD-L1 bicyclic peptide mimetics of the present invention comprise a stabilizing moiety or protecting moiety. The stabilizing moiety or protecting moiety may be coupled at any point on the peptide. Suitable stabilizing or protecting moieties include, but are not limited to, polyethylene glycol (PEG), a glycan or a capping moiety, including an acetyl group, pyroglutamate or an amino group. In preferred embodiments, the acetyl group and/or pyroglutamate are coupled to the N-terminal amino acid residue of the PD-L1 bicyclic peptide mimetic. In particular embodiments, the N-terminus of the PD-L1 bicyclic peptide mimetic is an acetamide. In preferred embodiments, the amino group is coupled to the C-terminal amino acid residue of the PD-L1 bicyclic peptide mimetic. In particular embodiments, the PD-L1 bicyclic peptide mimetic has a primary, secondary or tertiary amide, a hydrazide or a hydroxamide at the C-terminus; particularly a primary amide at the C-terminus. In preferred embodiments, the PEG is coupled to the N-terminal or C-terminal amino acid residue of the PD-L1 bicyclic peptide mimetic or through the amino group of a lysine side-chain or other suitably modified side-chain, especially through the N-terminal amino acid residue such as through the amino group of the residue, or through the amino group of a lysine side-chain.

In some preferred embodiments, the PD-L1 bicyclic peptide mimetics of the present invention have a primary amide or a free carboxyl group (acid) at the C-terminus and a primary amine or acetamide at the N-terminus.

### 2.4 Membrane Permeating Moieties

Although the PD-L1 bicyclic peptide mimetics of the invention may inherently permeate membranes, membrane permeation may further be increased by the conjugation of a membrane permeating moiety to the PD-L1 bicyclic peptide mimetic. Accordingly, in some embodiments, the PD-L1 bicyclic peptide mimetics of the present invention comprise a membrane permeating moiety. The membrane permeating moiety may be coupled at any point on the PD-L1 bicyclic peptide mimetic.

Suitable membrane permeating moieties include lipid moieties, cholesterol and proteins, such as cell penetrating peptides and polycationic peptides; especially lipid moieties.

Suitable cell penetrating peptides may include the peptides described in, for example, US 2009/0047272, US 2015/0266935 and US 2013/0136742. Accordingly, suitable cell penetrating peptides may include, but are not limited to, basic poly(Arg) and poly(Lys) peptides and basic poly(Arg) and poly(Lys) peptides containing non-natural analogues of Arg and Lys residues such as YGRKKRPQRRR (HIV TAT₄₇₋₅₇; SEQ ID NO: 22), RRWRRWWRRWWRRWRR (W/R; SEQ ID NO: 23), CWK₁₈ (AlkCWK₁₈; SEQ ID NO: 24), K₁₈WCCWK₁₈ (Di-CWK₁₈; SEQ ID NO: 25), WTLNSAGYLLGKINLKALAALAKKI L (Transportan; SEQ ID NO: 26), GLFEALEELWEAK (DipaLytic; SEQ ID NO: 27), K₁₆GGCRGDMFGCAK16RGD (K16RGD; SEQ ID NO: 28), K16GGCMFGCGG (PI; SEQ ID NO: 29), K₁₆ICRRARGDNPDDRCT (P2; SEQ ID NO: 30), KKWKMRRNQFWVKVQRbAK (B) bA (P3; SEQ ID NO: 31), VAYISRGGVSTYYSDTVKGRFTRQKYN KRA (P3a; SEQ ID NO: 32), IGRIDPANGKTKYAPKFQDKATRSNYYGNSPS (P9.3; SEQ ID NO: 33), KETWWETWWTEWSQPKKKRKV (Pep-1; SEQ ID NO: 34), PLAEIDGIELTY (Plae; SEQ ID NO: 35), K16GGPLAEIDGIELGA (Kplae; SEQ ID NO: 36), K₁₆GGPLAEIDGIELCA (cKplae; SEQ ID NO: 37), GALFLGFLGGAAGSTMGAWSQPKSKRKV (MGP; SEQ ID NO: 38), WEAK(LAKA)₂-LAKH(LAKA)₂LKAC (HA2; SEQ ID NO: 39), (LARL)₆NHCH₃ (LARL46; SEQ ID NO: 40), KLLKLLLKLWLLKLLL (Hel-11-7; SEQ ID NO: 41), (KKKK)₂GGC (KK; SEQ ID NO: 42), (KWKK)₂GCC (KWK; SEQ ID NO: 43), (RWRR)zGGC (RWR; SEQ ID NO: 44), PKKKRKV (SV40 NLS7; SEQ ID NO: 45), PEVKKKRKPEYP (NLS12; SEQ ID NO: 46), TPPKKKRKVEDP (NLS12a; SEQ ID NO: 47), GGGGPKKKRKVGG (SV40 NLS13; SEQ ID NO: 48), GGGFSTSLRARKA (AV NLS13; SEQ ID NO: 49), CKKKKKKSEDEYPYVPN (AV RME NLS17; SEQ ID NO: 50), CKKKKKKKSEDEYPYVPN FSTSLRARKA (AV FP NLS28; SEQ ID NO: 51), LVRKKRKTEEESPLKDKDAKKSKQE (SV40 N1 NLS24; SEQ ID NO: 52), and KgKzKgKgGGKg (Loligomer; SEQ ID NO: 53); HSV-1 tegument protein VP22; HSV-1 tegument protein VP22r fused with nuclear export signal (NES); mutant B-subunit of Escherichia coil enterotoxin EtxB (H57S); detoxified exotoxin A (ETA); the protein transduction domain of the HIV-1 Tat protein, GRKKRRQRRRPPQ (SEQ ID NO: 54); the Drosophila melanogaster Antennapedia domain Antp (amino acids 43-58), RQIKIWFQNRRMKWKK (SEQ ID NO: 55); Buforin II, TRSSRAGLQFPVGRVHRLLRK (SEQ ID NO: 56); hClock-(amino acids 35-47) (human Clock protein DNA-binding peptide), KRVSRNKSEKKRR (SEQ ID NO: 57); MAP (model amphipathic peptide), KIALKIALKALKAALKIA (SEQ ID NO: 58); K-FGF, AAVALLPAVLIALIAP (SEQ ID NO: 59); Ku70-derived peptide, comprising a peptide selected from the group comprising VPMLKE (SEQ ID NO: 60), VPMLK (SEQ ID NO: 61), PMLKE (SEQ ID NO: 62) or PMLK (SEQ ID NO: 63); Prion, Mouse Prpe (amino acids 1-28), MANLGYWLIALFVTMWTDVGLCKKRPKP (SEQ ID NO: 64); pVEC, LLIILRRRIRKQAHAHSK (SEQ ID NO: 65); Pep-I, KETWWETWWTEWSQPKKKRKV (SEQ ID NO: 66); SynBI, RGGRLSYSRRRFSTSTGR (SEQ ID NO: 67); Transportan, GWTLNSAGYLLGKINLKAIAAIAKKIL (SEQ ID NO: 68); Transportan-10, AGYLLGKINLKALAALAKKIL (SEQ ID NO: 69); CADY, Ac-GLWRALWRLLRSLWRLLWRA-cysteamide (SEQ ID NO: 70); Pep-7, SDLWEMMMVSIACQY (SEQ ID NO: 71); HN-1, TSPLNIHNGQKL (SEQ ID NO: 72); VT5, DPKGDPKGVTVTVTVTVTGKGDPKPD (SEQ ID NO: 73); or pISL, RVIRVWFQNKRCKDKK (SEQ ID NO: 74).

In preferred embodiments, the membrane permeating moiety is a lipid moiety, such as a C₁₀-C₂₀ fatty acyl group, especially stearoyl (octadecanoyl; C₁₈), palmitoyl (hexadecanoyl; C₁₆) or myristoyl (tetradecanoyl; C₁₄); most especially myristoyl. In preferred embodiments, the membrane permeating moiety is coupled to the N- or C-terminal amino acid residue or through the amino group of a lysine side-chain of the PD-L1 bicyclic peptide mimetic or other suitably modified side-chain, especially the N-terminal amino add residue of the PD-L1 bicyclic peptide mimetic or through the amino group of a lysine side-chain. In particular embodiments, the membrane permeating moiety is coupled through the amino group of the N-terminal amino acid residue.

In some specific embodiments, the membrane permeating moiety is a myristoyl, coupled to the N-terminal amino acid residue.

### 2.5 Targeting Peptides

In some embodiments, the PD-L1 bicyclic peptide mimetics further comprise a targeting peptide capable of enhancing transport of the molecule across the blood-brain barrier (“BBB”) or into particular cell types. In certain embodiments, the targeting peptide has a sequence of Angiopep-1 (SEQ ID NO: 100); Angiopep-2 (SEQ ID NO: 101); Angiopep-3 (SEQ ID NO: 104); Angiopep-4a (SEQ ID NO: 105); Angiopep4b (SEQ ID NO: 106); Angiopep-5 (SEQ ID NO: 107); Angiopep-6 (SEQ ID NO: 108); or Angiopep-7 (SEQ ID NO: 109) (see TABLE 4). On conjugation with the targeting peptide, the PD-L1 bicyclic peptide mimetic may be efficiently transported into a particular cell type (e.g., any one, two , three, four, or five of liver, lung, kidney, spleen, and muscle) or may cross the mammalian BBB efficiently (e.g., Angiopep-1, -2, -3, -4a, -4b, -5, and -6). In some alternative embodiments, on conjugation with the targeting peptide, the PD-L1 bicyclic peptide mimetic may be efficiently transported into a particular cell type (e.g., any one, two , three, four, or five of liver, lung, kidney, spleen, and muscle) but does not cross the mammalian BBB efficiently (e.g., Angiopep-7). The targeting peptide may be of any length, for example, at least 6, 7, 8, 9, 10, 11, 12, 13, 14, 15, 16, 17, 18, 19, 20, 21, 25, 35, 50, 70 or 100 amino acids, or any range between these numbers. In certain embodiments, the peptide vector is 10 to 50 amino acids in length.

TABLE 4
Exemplary Targeting Peptide Sequences

| Peptide | Amino Acid Sequence | SEQ ID NO: |
|---|---|---|
| Angiopep-1 | TFFYGGCRGKRNNFKTEEY | 100 |
| Angiopep-2 | TFFYGGSRGKRNNFKTEEY | 101 |
| Angiopep-3 | TFFYGGSRGKRNNFKTEEY | 104 |
| Angiopep-4a | RFFYGGSRGKRNNFKTEEY | 105 |
| Angiopep-4b | RFFYGGSRGKRNNFKTEEY | 106 |
| Angiopep-5 | RFFYGGSRGKRNNFRTEEY | 107 |
| Angiopep-6 | TFFYGGSRGKRNNFRTEEY | 108 |
| Angiopep-7 | TFFYGGSRGRRNNFRTEEY | 109 |
| Cys-Angiopep-2 | CTFFYGGSRGKRNNFKTEEY | 110 |
| Angiopep-2-Cys | TFFYGGSRGKRNNFKTEEYC | 111 |

In instances where the PD-L1 bicycle peptide mimetic is used to treat or prevent indications wherein it must cross the BBB (e.g., brain cancer or other cancer protected by the BBB), the targeting peptide may comprise, consist, or consist essentially of an amino acid sequence selected from the group consisting of Angiopep-2 (SEQ ID NO:101), Angiopep-1 (SEQ ID NO:100), cys-Angiopep-2 (SEQ ID NO:110), and Angiopep-2-cys (SEQ ID NO:111). In preferred embodiments, the targeting peptide is Angiopep-2 (SEQ ID NO: 101).

In some embodiments, the targeting peptide may be coupled at any point on PD-L1 bicyclic peptide mimetic, especially to the N- or C-terminal amino acid residues. In alternative embodiments, the targeting peptide may be conjugated to the Cys linker regions of the PD-L1 bicyclic peptide mimetics.

Accordingly, in another aspect of the present invention, there is provided an isolated or purified PD-L1 bicyclic peptide mimetic represented by Formula II:

M-P (Formula II)
wherein:
M is a membrane permeating moiety; and
P is an isolated or purified PD-L1 bicyclic peptide mimetic represented by Formula I.

In some embodiments, M is coupled at any point on the PD-L1 bicyclic peptide mimetic; especially to the N- or C-terminal amino acid residue or through the amino group of a lysine side-chain of the PD-L1 bicyclic peptide mimetic or other suitably modified side-chain, more especially the N-terminal amino acid residue of the PD-L1 bicyclic peptide mimetic or through the amino group of a lysine side-chain; most especially through the amino group of the N-terminal amino acid residue.

Suitable membrane permeating moieties and embodiments of the PD-L1 bicyclic peptide mimetic represented by Formula I are as described herein.

The PD-L1 bicyclic peptide mimetics of the present invention may be in the form of salts or prodrugs. The salts of the PD-L1 bicyclic peptide mimetics of the present invention are preferably pharmaceutically acceptable, but it will be appreciated that non-pharmaceutically acceptable salts also fall within the scope of the present invention.

The PD-L1 bicyclic peptide mimetics of the present invention may be in crystalline form and/or in the form of solvates, for example, hydrates. Solvation may be performed using methods known in the art.

The peptides of the present invention may be prepared using recombinant DNA techniques or by chemical synthesis.

### 2.6 Nucleic Acid Molecules

In some embodiments, the PD-L1 bicyclic peptide mimetics of the present invention are prepared using recombinant DNA techniques. For example, the PD-L1 bicyclic peptide mimetics of the invention may be prepared by a procedure including the steps of: (a) preparing a construct comprising a polynucleotide sequence that encodes the PD-L1 bicyclic peptide mimetic of the invention and that is operably linked to a regulatory element; (b) introducing the construct into a host cell; (c) culturing the host cell to express the polynucleotide sequence to thereby produce the encoded PD-L1 bicyclic peptide mimetic of the invention; and (d) isolating the PD-L1 bicyclic peptide mimetic of the invention from the host cell. The PD-L1 bicyclic peptide mimetics of the present invention may be prepared recombinantly using standard protocols, for example, as described in Klint, et al. (2013) PLOS One, 8(5): e63865; Sambrook, et al. (1989) Molecular Cloning: A Laboratory Manual (Cold Spring Harbour Press), in particular Sections 16 and 17; Ausubel, et al. (1998) Current Protocols in Molecular Biology (John Wiley and Sons, Inc.), in particular Chapters 10 and 16; Coligan, et al. (1997) Current Protocols in Protein Science (John Wiley and Sons, Inc.), in particular Chapters 1, 5 and 6; and U.S. Pat. No. 5,976,567, the entire contents of which are hereby incorporated by reference.

Thus, the present invention also contemplates nucleic acid molecules which encode a PD-L1 bicyclic peptide mimetic of the invention. Thus, in a further aspect of the present invention, there is provided an isolated nucleic acid molecule comprising a polynucleotide sequence that encodes the PD-L1 bicyclic peptide mimetic of the invention or is complementary to a polynucleotide sequence that encodes a PD-L1 bicyclic peptide mimetic of the invention, such as the PD-L1 bicycle peptide mimetic of Formula I, of SEQ ID NO: 1, or variant PD-L1 bicyclic peptide mimetic as described herein.

The isolated nucleic acid molecules of the present invention may be DNA or RNA. When the nucleic acid molecule is in DNA form, it may be genomic DNA or cDNA. RNA forms of the nucleic acid molecules of the present invention are generally mRNA.

Although the nucleic acid molecules are typically isolated, in some embodiments, the nucleic acid molecules may be integrated into or ligated to or otherwise fused or associated with other genetic molecules, such as an expression vector. Generally, an expression vector includes transcriptional and translational regulatory nucleic acid operably linked to the polynucleotide sequence. Accordingly, in another aspect of the invention, there is provided an expression vector comprising a polynucleotide sequence that encodes a PD-L1 bicyclic peptide mimetic of the invention, such as the PD-L1 bicyclic peptide mimetic of Formula I, of SEQ ID NO: 1, or variant PD-L1 bicyclic peptide mimetic as described herein.

Typical vectors contain transcription and translation terminators, transcription and translation initiation sequences and promoters useful for regulation of the expression of the nucleic acid. The vectors optionally comprise generic expression cassettes containing at least one independent terminator sequence, sequences permitting replication of the cassette in eukaryotes, prokaryotes or both, (e.g., shuttle vectors) and selection markers for both prokaryotic and eukaryotic systems. Vectors may be suitable for replication and integration in prokaryotes, eukaryotes, or both. See, Giliman and Smith (1979), Gene, 8: 81-97; Roberts et al. (1987) Nature, 328: 731-734; Berger and Kimmel, Guide to Molecular Cloning Techniques, Methods in Enzymology, volume 152, Academic Press, Inc., San Diego, Calif. (Berger); Sambrook et al. (1989), Molecular Cloning - a Laboratory Manual (2nd ed.) Vol. 1-3, Cold Spring Harbor Laboratory, Cold Spring Harbor Press, N.Y.; and Ausubel et al. (1994) Current Protocols in Molecular Biology, eds., Current Protocols, a Joint venture between Greene Publishing Associates, Inc. and John Wiley & Sons, Inc. (Supplement), the entire contents of which are incorporated by reference.

Expression vectors containing regulatory elements from eukaryotic viruses such as retroviruses are typically used for expression of nucleic acid sequences in eukaryotic cells. SV40 vectors include pSVT7 and pMT2. Vectors derived from bovine papilloma virus include pBV-IMTHA, and vectors derived from Epstein Bar Virus include pHEBO, and p205. Other exemplary vectors include pMSG, pAV009/A+, pMT010/A+, pMAMneo-5, baculovirus pDSVE, and any other vector allowing expression of proteins under the direction of the SV-40 early promoter, SV-40 later promoter, metallothionein promoter, murine mammary tumour virus promoter, Rous sarcoma virus promoter, polyhedrin promoter or other promoters shown effective for expression in eukaryotic cells.

While a variety of vectors may be used, it should be noted that viral expression vectors are useful for modifying eukaryotic cells because of the high efficiency with which the viral vectors transfect target cells and integrate into the target cell genome. Illustrative expression vectors of this type can be derived from viral DNA sequences including, but not limited to, adenovirus, adeno-associated viruses, herpes-simplex viruses and retroviruses such as B, C, and D retroviruses as well as spumaviruses and modified lentiviruses. Suitable expression vectors for transfection of animal cells are described, for example, by Wu and Ataai (2000) Curr. Opln. Biotechnol., 11(2): 205-208; Vigna and Naldini (2000) J. Gene Med., 2(5): 308-316; Kay et al. (2001) Nat. Med., 7(1): 33-40; Athanasopoulos et al. (2000) Int. J. Mol. Med., 6(4): 363-375; and Walther and Stein (2000) Drugs, 60(2): 249-271, the entire contents of which are incorporated by reference.

The polypeptide or peptide-encoding portion of the expression vector may comprise a naturally-occurring sequence or a variant thereof, which has been engineered using recombinant techniques. In one example of a variant, the codon composition of a polynucleotide encoding a PD-L1 bicyclic peptide mimetic of the invention is modified to permit enhanced expression of the PD-L1 bicyclic peptide mimetic of the invention in a mammalian host using methods that take advantage of codon usage bias, or codon translational efficiency in specific mammalian cell or tissue types as set forth, for example, in International Publications WO 99/02694 and WO 00/42215. Briefly, these latter methods are based on the observation that translational efficiencies of different codons vary between different cells or tissues and that these differences can be exploited, together with codon composition of a gene, to regulate expression of a protein in a particular cell or tissue type. Thus, for the construction of codon-optimized polynucleotides, at least one existing codon of a parent polynucleotide is replaced with a synonymous codon that has a higher translational efficiency in a target cell or tissue than the existing codon it replaces. Although it is preferable to replace all the existing codons of a parent nucleic acid molecule with synonymous codons which have that higher translational efficiency, this is not necessary because increased expression can be accomplished even with partial replacement. Suitably, the replacement step affects 5%, 10%, 15%, 20%, 25%, 30%, more preferably 35%, 40%, 50%, 60%, 70% or more of the existing codons of a parent polynucleotide.

The expression vector is compatible with the cell in which it is introduced such that the PD-L1 bicyclic peptide mimetic of the invention is expressible by the cell. The expression vector is introduced into the cell by any suitable means which will be dependent on the particular choice of expression vector and cell employed. Such means of introduction are well-known to those skilled in the art. For example, introduction can be effected by use of contacting (e.g. in the case of viral vectors), electroporation, transformation, transduction, conjugation or triparental mating, transfection, infection membrane fusion with cationic lipids, high-velocity bombardment with DNA-coated microprojectiles, incubation with calcium phosphate-DNA precipitate, direct microinjection into single cells, and the like. Other methods also are available and are known to those skilled in the art. Alternatively, the vectors are introduced by means of cationic lipids, e.g., liposomes. Such liposomes are commercially available (e.g., LIPOFECTIN®, LIPOFECTAMINE™, and the like, supplied by Life Technologies, Gibco BRL, Gaithersburg, Md.).

### Molecular Scaffolds

The PD-L1 bicyclic peptide mimetics of the invention comprise, consist essentially of, or consist of, the polypeptide covalently bound to a molecular scaffold. Molecular scaffolds are described in, for example, International PCT Patent Publication No. WO 2009/098450 and references cited therein, particularly WO 2004/077062 and WO 2006/078161. As noted in the foregoing documents, the molecular scaffold may be a small molecule, such as a small organic molecule.

In some embodiments, the molecular scaffold may be, or may be based on, natural monomers such as nucleosides, sugars, or steroids. For example, the molecular scaffold may comprise a short polymer of such entities, such as a dimer or trimer.

In one embodiment, the molecular scaffold is a compound of known toxicity, for example, low toxicity. Examples of suitable compounds include cholesterols, nucleotides, steroids, or existing drugs such as temazepam.

In some embodiments, the molecular scaffold may be a macromolecule. In some embodiments, the molecular scaffold is a macromolecule composed of amino acids, nucleotides, or carbohydrates.

In some embodiments, the molecular scaffold comprises reactive groups that are capable of reacting with functional group(s) of the polypeptide to form covalent bonds.

The molecular scaffold may comprise chemical groups, such as amines, thiols, alcohols, ketones, aldehydes, nitriles, carboxylic acids, esters, alkenes, alkynes, azides, anhydrides, succinimides, maleimides, alkyl halides and acyl halides.

In some embodiments, the scaffold is an aromatic molecular scaffold (i.e., a scaffold comprising a (hetero)aryl group). These aromatic rings can optionally contain one or more heteroatoms (e.g., one or more of N, O, S, and P), such as thienyl rings, pyridyl rings, and furanyl rings. The aromatic rings can be optionally substituted. The aryl rings can also be optionally substituted. Suitable substituents include alkyl groups (which can optionally be substituted), other aryl groups (which may themselves be substituted), heterocyclic rings (saturated or unsaturated), alkoxy groups (which is meant to include aryloxy groups (e.g., phenoxy groups)), hydroxy groups, aldehyde groups, nitro groups, amine groups (e.g., unsubstituted, or mono- or di-substituted with aryl or alkyl groups), carboxylic acid groups, carboxylic acid derivatives (e.g., carboxylic acid esters, amides, etc.), halogen atoms (e.g., Cl, Br, and I), and the like.

Suitably, the scaffold comprises a tris-substituted (hetero)aromatic or (hetero)alicyclic moiety, for example a tris-methylene substituted (hetero)aromatic or (hetero)alicyclic moiety. The (hetero)aromatic or (hetero)alicyclic moiety is suitably a six-membered ring structure, preferably tris-substituted such that the scaffold has a 3-fold symmetry axis.

In some embodiments, the scaffold is a tris-methylene (hetero)aryl moiety, for example a 1,3,5-tris methylene benzene moiety. In these embodiments, the corresponding scaffold molecule suitably has a leaving group on the methylene carbons. The methylene group then forms the R1moiety of the alkylamino linkage as defined herein. In these methylene-substituted (hetero)aromatic compounds, the electrons of the aromatic ring can stabilize the transition state during nucleophilic substitution. Thus, for example, benzyl halides are 100-1000 times more reactive towards nucleophilic substitution than alkyl halides that are not connected to a (hetero)aromatic group.

In embodiments of this type, the scaffold and scaffold molecule have the general formula:

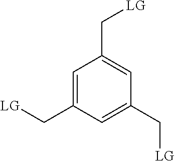

Where LG represents a leaving group as described further below for the scaffold molecule, or LG (including the adjacent methylene group forming the R₁ moiety of the alkylamino group) represents the alkylamino linkage to the peptide in the conjugates of the invention.

In some embodiments, the group LG above may be a halogen such as, but not limited to, a bromine atom, in which case the scaffold molecule is 1,3,5-Tris(bromomethyl)benzene (TBMB). Another suitable molecular scaffold molecule is 2,4,6-tris(bromomethyl) mesitylene. It is similar to 1,3,5-tris(bromomethyl) benzene but contains additionally three methyl groups attached to the benzene ring. In the case of this scaffold, the additional methyl groups may form further contacts with the peptide and hence add additional structural constraint. Thus, a different diversity range is achieved than with 1,3,5-Tris(bromomethyl)benzene.

Another preferred molecule for forming the scaffold for reaction with the peptide by nucleophilic substitution is 1,3,5-tris(bromoacetamido)benzene (TBAB):

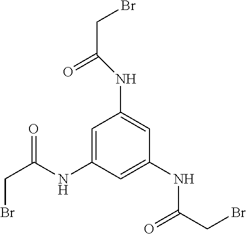 [SMILES: O=C(CBr)Nc1cc(NC(=O)CBr)cc(NC(=O)CBr)c1]

In some alternative embodiments, the scaffold is a non-aromatic molecular scaffold (e.g., a scaffold comprising a (hetero)alicyclic group). As used herein, “(hetero)alicyclic” refers to a homocyclic or heterocyclic saturated ring. The ring can be unsubstituted, or it can be substituted with one or more substituents. The substituents can be saturated or unsaturated, aromatic or nonaromatic, and examples of suitable substituents include those recited above in the discussion relating to substituents on alkyl and aryl groups. Furthermore, two or more ring substituents can combine to form another ring, so that “ring”, as used herein, is meant to include fused ring systems. In these embodiments, the alicyclic scaffold is preferably 1,1′,1″-(1,3,5-triazinane-1,3,5-triyl)triprop-2-en-1-one (TATA).

In some alternative embodiments the molecular scaffold may have a tetrahedral geometry such that reaction of four functional groups of the encoded peptide with the molecular scaffold generates not more than two product isomers. Other geometries are also possible; indeed, an almost infinite number of scaffold geometries is possible, leading to greater possibilities for peptide ligand diversification.

The peptides used to form the bicyclic peptides of the invention comprise cysteines that are used to form thioether bonds to the scaffold, with replacement of the terminal —SH group of cysteine by —NH₂.

The bicyclic peptides of the present invention have a number of advantageous properties which enable them to be considered as beneficial drug-like molecules for injection, inhalation, nasal, ocular, oral or topical administration. Such advantageous properties include:
species cross-reactivity, which is a typical requirement for preclinical pharmacodynamics and pharmacokinetic evaluation;
protease stability, as bicyclic peptide ligands ideally demonstrate stability to plasma proteases, epithelial (“membrane-anchored”) proteases, gastric and intestinal proteases, lung surface proteases, intracellular proteases, and the like. Protease stability should be maintained between different species such that a bicycle peptide candidates can be developed in animal models as well as administered with confidence to humans;
desirable solubility profile, which is a function of the proportion of charged and hydrophilic versus hydrophobic residues and intra/inter-molecular H-bonding, which is important for formulation and absorption purposes; and
an optimal elimination half-life. Depending upon the clinical indication and treatment regimen, it may be required to develop a bicyclic peptide for short exposure in an acute illness management setting, or develop a bicyclic peptide with enhanced retention. It is therefore optimal for the management of more chronic disease states and cancers. Other factors driving the desirable elimination half-life are requirements of sustained exposure for maximal therapeutic efficiency versus the accompanying toxicology due to sustained exposure of the agent.

In one embodiment, the molecular scaffold may comprise or may consist of tris(bromomethyl)benzene, especially 1,3,5-tris(bromomethyl)benzene (“TBMB”), or a derivative thereof.

In some particularly preferred embodiments, the molecular scaffold is 1,3,5-(tribromomethyl)benzene).

In some other embodiments, the molecular scaffold is 2,4,6-tris(bromomethyl)mesitylene. This molecule is similar to 1,3,5-tris(bromomethyl)benzene but contains three additional methyl groups attached to the benzene ring. This has the advantage that the additional methyl groups may form further contacts with the polypeptide and hence add additional structural constraint.

Scaffold reactive groups that could be used on the molecular scaffold to react with thiol groups of cysteines are alkyl halides (or also names halogenoalkanes or haloalkanes). Examples include bromomethylbenzene (the scaffold reactive group exemplified by TBMB) or iodoacetamide. Other scaffold reactive groups that are used to selectively couple compounds to cysteines in proteins are maleimides. Examples of maleimides which may be used as molecular scaffolds in the invention include: tris-(2-maleimidoethyl)amine, tris-(2-maleimidoethyl)benzene, tris-(maleimido)benzene. Selenocysteine is also a natural amino acid which has a similar reactivity to cysteine and can be used for the same reactions. Thus, wherever cysteine is mentioned, it is typically acceptable to substitute selenocysteine unless the context suggests otherwise.

### 2.7 Synthesis

The peptides of the present invention may be manufactured synthetically by standard techniques followed by reaction with a molecular scaffold in vitro. When this is performed, standard chemistry may be used. This enables the rapid large scale preparation of soluble material for further downstream experiments or validation. Such methods could be accomplished using conventional chemistry such as that disclosed in Timmerman et al. (supra).

Thus, the invention also relates to manufacture of polypeptides or conjugates selected as set out herein, wherein the manufacture comprises optional further steps as explained below. In one embodiment, these steps are carried out on the end product polypeptide/conjugate made by chemical synthesis.

Optionally amino acid residues in the polypeptide of interest may be substituted when manufacturing a conjugate or complex.

Peptides can also be extended, to incorporate for example another loop and therefore introduce multiple specificities. To extend the peptide, it may simply be extended chemically at its N-terminus or C-terminus or within the loops using orthogonally protected lysines (and analogues) using standard solid phase or solution phase chemistry. Standard protein chemistry may be used to introduce an activatable N- or C-terminus. Alternatively, additions may be made by fragment condensation or native chemical ligation e.g. as described in (Dawson et al., 1994. Synthesis of Proteins by Native Chemical Ligation. Science 266:776-779), or by enzymes, for example using subtiligase as described in (Chang et al., Proc Natl Acad Sci U S A. 1994 Dec 20; 91 (26): 12544-8 or in Hikari et al Bioorganic & Medicinal Chemistry Letters Volume 18, Issue 22, 15 November 2008, pages 6000-6003).

Alternatively, the peptides may be extended or modified by further conjugation through disulphide bonds. This has the additional advantage of allowing the first and second peptide to dissociate from each other once within the reducing environment of the cell. In this case, the molecular scaffold (e.g., TBMB) could be added during the chemical synthesis of the first peptide so as to react with the three cysteine groups; a further cysteine could then be appended to the N-terminus of the first peptide, so that this cysteine only reacted with a free cysteine of the second peptide.

Similar techniques apply equally to the synthesis/coupling of two bicyclic and bispecific macrocycles, potentially creating a tetraspecific molecule. Furthermore, addition of other functional groups or effector groups may be accomplished in the same manner, using appropriate chemistry, coupling at the N- or C-termini or via side chains. In one embodiment, the coupling is conducted in such a manner that it does not block the activity of either entity.

In some embodiments, the PD-L1 bicyclic peptide mimetics of the invention may be produced inside a cell by introduction of one or more expression constructs, such as an expression vector, that comprise a polynucleotide sequence that encodes a PD-L1 bicyclic peptide mimetic of the invention.

The invention contemplates recombinantly producing the PD-L1 bicyclic peptide mimetic of the invention inside a host cell, such as a mammalian cell (e.g., Chinese hamster ovary (CHO) cell, mouse myeloma (NSO) cell, baby hamster kidney (BHK) cell or human embryonic kidney (HEK293) cell), yeast cell (e.g., Pichia pastoris cell, Saccharomyces cerevisiae cell, Schizosaccharomyces pombe cell, Hansenula polymorpha cell, Kluyveromyces lactis cell, Yarrowia lipolytica cell or Arxula adeninivorans cell), or bacterial cell (e.g., Escherichia coil cell, Corynebacterium glutamicum or Pseudomonas fluorescens cell).

For therapeutic applications, the invention also contemplates producing the PD-L1 bicyclic peptide mimetics of the invention in vivo inside a cell of a subject, for example a PD-L1 overexpressing cell, such as a vertebrate cell, particularly a mammalian or avian cell, especially a mammalian cell.

In some embodiments, the PD-L1 bicyclic peptide mimetics of the present invention are prepared using standard peptide synthesis methods, such as solution synthesis or solid phase synthesis. The chemical synthesis of the PD-L1 bicyclic peptide mimetics of the invention may be performed manually or using an automated synthesizer. For example, the linear peptides may be synthesized using solid phase peptide synthesis using either Boc or Fmoc chemistry, as described in Merrifield (1963) J Am Chem Soc, 85(14): 2149-2154; Schnolzer, et al. (1992) Int J Pept Protein Res, 40: 180-193 and Cardoso, et al. (2015) Mol Pharmacol, 88(2): 291-303, the entire contents of which are incorporated by reference. Following deprotection and cleavage from the solid support, the linear peptides are purified using suitable methods, such as preparative chromatography.

### 3. PHARMACEUTICAL COMPOSITIONS

In accordance with the present invention, the PD-L1 bicyclic peptide mimetics are useful in compositions and methods for the treatment or prevention of a condition involving the nuclear localization of PD-L1, for example a cancer.

Thus, in some embodiments, the PD-L1 bicyclic peptide mimetic of the present invention may be in the form of a pharmaceutical composition, wherein the pharmaceutical composition comprises a PD-L1 bicyclic peptide mimetic of the invention and a pharmaceutically acceptable carrier or diluent.

The PD-L1 bicyclic peptide mimetics of the invention may be formulated into the pharmaceutical compositions as neutral or salt forms.

As will be appreciated by those skilled in the art, the choice of pharmaceutically acceptable carrier or diluent will be dependent on the route of administration and on the nature of the condition and the subject to be treated. The particular carrier or delivery system and route of administration may be readily determined by a person skilled in the art. The carrier or delivery system and route of administration should be carefully selected to ensure that the activity of the PD-L1 bicyclic peptide mimetic is not depleted during preparation of the formulation and the PD-L1 bicyclic peptide mimetic is able to reach the site of action intact. The pharmaceutical compositions of the invention may be administered through a variety of routes including, but not limited to, oral, rectal, topical, intranasal, intraocular, transmucosal, intestinal, enteral, intramuscular, subcutaneous, intramedullary, intrathecal, intraventricular, intracerebral, intravaginal, intravesical, intravenous or intraperitoneal administration.

The pharmaceutical forms suitable for injectable use include sterile injectable solutions or dispersions and sterile powders for the preparation of sterile injectable solutions. Such forms should be stable under the conditions of manufacture and storage and may be preserved against reduction, oxidation and microbial contamination.

A person skilled in the art will readily be able to determine appropriate formulations for the PD-L1 bicyclic peptide mimetics of the invention using conventional approaches. Techniques for formulation and administration may be found in, for example, Remington (1980) Remington's Pharmaceutical Sciences, Mack Publishing Co., Easton, Pa., latest edition; and Niazi (2009) Handbook of Pharmaceutical Manufacturing Formulations, Informa Healthcare, New York, second edition, the entire contents of which are incorporated by reference.

Identification of preferred pH ranges and suitable excipients, such as antioxidants, is routine in the art, for example, as described in Katdare and Chaubel (2006) Excipient Development for Pharmaceutical, Biotechnology and Drug Delivery Systems (CRC Press). Buffer systems are routinely used to provide pH values of a desired range and may include, but are not limited to, carboxylic acid buffers, such as acetate, citrate, lactate, tartrate and succinate; glycine; histidine; phosphate; tris(hydroxymethyl)aminomethane (Tris); arginine; sodium hydroxide; glutamate; and carbonate buffers. Suitable antioxidants may include, but are not limited to, phenolic compounds such as butylated hydroxytoluene (BHT) and butylated hydroxyanisole; vitamin E; ascorbic acid; reducing agents such as methionine or sulfite; metal chelators such as ethylene diamine tetraacetic acid (EDTA); cysteine hydrochloride; sodium bisulfite; sodium meta bisulfite; sodium sulfite; ascorbyl palmitate; lecithin; propyl gallate; and alpha-tocopherol.

For injection, the PD-L1 bicyclic peptide mimetics of the invention may be formulated in aqueous solutions, suitably in physiologically compatible buffers such as Hanks' solution, Ringer's solution or physiological saline buffer. For transmucosal administration, penetrants appropriate to the barrier to be permeated are used in the formulation. Such penetrants are generally known in the art.

The compositions of the present invention may be formulated for administration in the form of liquids, containing acceptable diluents (such as saline and sterile water), or may be in the form of lotions, creams or gels containing acceptable diluents or carriers to impart the desired texture, consistency, viscosity and appearance. Acceptable diluents and carriers are familiar to those skilled in the art and include, but are not restricted to, ethoxylated and nonethoxylated surfactants, fatty alcohols, fatty acids, hydrocarbon oils (such as palm oil, coconut oil, and mineral oil), cocoa butter waxes, silicon oils, pH balancers, cellulose derivatives, emulsifying agents such as non-ionic organic and inorganic bases, preserving agents, wax esters, steroid alcohols, triglyceride esters, phospholipids such as lecithin and cephalin, polyhydric alcohol esters, fatty alcohol esters, hydrophilic lanolin derivatives and hydrophilic beeswax derivatives.

Alternatively, the PD-L1 bicyclic peptide mimetics of the present invention can be formulated readily using pharmaceutically acceptable carriers well known in the art into dosages suitable for oral administration, which is also contemplated for the practice of the present invention. Such carriers enable the bioactive agents of the invention to be formulated in dosage forms such as tablets, pills, capsules, liquids, gels, syrups, slurries, suspensions and the like, for oral ingestion by a patient to be treated. These carriers may be selected from sugars, starches, cellulose and its derivatives, malt, gelatin, talc, calcium sulfate, vegetable oils, synthetic oils, polyols, alginic acid, phosphate buffered solutions, emulsifiers, isotonic saline and pyrogen-free water.

Pharmaceutical formulations for parenteral administration include aqueous solutions of the PD-L1 bicyclic peptide mimetics of the invention in water-soluble form. Additionally, suspensions of the PD-L1 bicyclic peptide mimetics of the invention may be prepared as appropriate oily injection suspensions. Suitable lipophilic solvents or vehicles include fatty oils such as sesame oil, or synthetic fatty acid esters, such as ethyl oleate or triglycerides. Aqueous injection suspensions may contain substances that increase the viscosity of the suspension, such as sodium carboxymethyl cellulose, sorbitol or dextran. Optionally, the suspension may also contain suitable stabilizers or agents that increase the solubility of the compounds to allow for the preparation of highly concentrated solutions.

Sterile solutions may be prepared by combining the active compounds in the required amount in the appropriate solvent with other excipients as described above as required, followed by sterilization, such as filtration. Generally, dispersions are prepared by incorporating the various sterilized active compounds into a sterile vehicle which contains the basic dispersion medium and the required excipients as described above. Sterile dry powders may be prepared by vacuum- or freeze-drying a sterile solution comprising the active compounds and other required excipients as described above.

Pharmaceutical preparations for oral use can be obtained by combining the PD-L1 bicyclic peptide mimetics of the invention with solid excipients and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Such compositions may be prepared by any of the methods of pharmacy but all methods include the step of bringing into association one or more therapeutic agents as described above with the carrier which constitutes one or more necessary ingredients. In general, the pharmaceutical compositions of the present invention may be manufactured in a manner that is itself known, e.g. by means of conventional mixing, dissolving, granulating, dragee-making, levigating, emulsifying, encapsulating, entrapping or lyophilizing processes.

Dragee cores are provided with suitable coatings. For this purpose, concentrated sugar solutions may be used, which may optionally contain gum arable, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of particle doses.

Pharmaceuticals which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added.

The PD-L1 bicyclic peptide mimetics of the invention may be incorporated into modified-release preparations and formulations, for example, polymeric microsphere formulations, and oil- or gel-based formulations.

In particular embodiments, the PD-L1 bicyclic peptide mimetic of the invention may be administered in a local rather than systemic manner, such as by injection of the PD-L1 bicyclic peptide mimetic directly into a tissue, which is preferably subcutaneous or omental tissue, often in a depot or sustained release formulation. In cases of local administration or selective uptake, the effective local concentration of the agent may not be related to plasma concentration.

### 3.1 Drug Delivery Systems

Furthermore, the PD-L1 bicyclic peptide mimetic of the invention may be administered in a targeted drug delivery system, such as in a particle which is suitably targeted to and taken up selectively by a cell or tissue. In some embodiments, the PD-L1 bicyclic peptide mimetic of the invention is contained in or otherwise associated with a vehicle selected from liposomes, micelles, dendrimers, biodegradable particles, artificial DNA nanostructure, lipid-based nanoparticles and carbon or gold nanoparticles. In illustrative examples of this type, the vehicle is selected from poly(lactic acid) (PLA), poly(glycolic acid) (PGA), poly(lactic-co-glycolic acid) (PLGA), polyethylene glycol) (PEG), PLA-PEG copolymers and combinations thereof.

### 3.1.1 Lipid Nanoparticles

In some preferred embodiments, the PD-L1 bicyclic peptide mimetic is complexed, encapsulated, partially encapsulated, or associated with one or more lipids (e.g., cationic lipids and/or ionisable lipids and/or neutral lipids), thereby forming lipid-based carriers such as liposomes, lipid nanoparticles, lipoplexes and/or nanoliposomes. The PD-L1 bicyclic peptide mimetic may be completely or partially located in the interior space of the lipid-based carrier. The incorporation of therapeutic agents (e.g., peptides, nucleic acids, and small molecules) into lipid-carriers is also referred to herein as “encapsulation” wherein the therapeutic agent (e.g. the PD-L1 bicyclic peptide mimetic) is entirely contained within the interior space of the lipid-carrier. One advantage for incorporating the PD-L1 bicyclic peptide mimetic into the lipid-carrier is to protect the PD-L1 bicyclic peptide mimetic from an environment which is likely to contain enzymes or chemicals, or conditions that degrade nucleic acid and/or systems or receptors that cause the rapid excretion of the peptide. Moreover, incorporating the PD-L1 bicyclic peptide mimetic into lipid-based carriers may promote the uptake of the PD-L1 bicyclic peptide mimetic, and hence, may enhance the therapeutic effect of the peptide. Accordingly, incorporating a PD-L1 bicyclic peptide mimetic into liposomes, lipid nanoparticles, lipoplexes, and/or nanoliposomes may be particularly suitable for compositions of the present invention (e.g., for intramuscular, intravenous, and/or intradermal administration).

In this context, the terms “complexed” or “associated” refer to the essentially stable combination of nucleic acid with one or more lipids into larger complexes or assemblies without covalent binding.

Lipid nanoparticles are suitably characterised as microscopic vesicles having an interior aqua space sequestered from an outer medium by a membrane or one or more bilayers. Bilayer membranes of lipid nanoparticles are typically formed by amphiphilic molecules, such as lipids of synthetic or natural origin that comprise spatially separated hydrophilic and hydrophobic domains. Bilayer membranes of the liposomes can also be formed by amphophilic polymers and surfactants (e.g., polymerosomes, niosomes, etc.). In the context of the present invention, a lipid nanoparticle typically serves to transport the PD-L1 bicyclic peptide mimetic to a target tissue (e.g., a tumour).

In some embodiments, a lipid nanoparticle may comprise an ionizable lipid. As used herein, the term “ionizable lipid” has its ordinary meaning in the art and may refer to a lipid comprising one or more charged moieties. In some embodiments, an ionizable lipid may be positively charged or negatively charged. An ionizable lipid may be positively charged, in which case it can be referred to as a “cationic lipid”. In certain embodiments, an ionizable lipid molecule may comprise an amine group, and can be referred to as an ionizable amino lipid. As used herein, a “charged moiety” is a chemical moiety that carries a formal electronic charge, e.g., monovalent (+1, or −1), divalent (+2, or −2), trivalent (+3, or −3), etc. The charged moiety may be anionic (i.e., negatively charged) or cationic (i.e., positively charged). Examples of positively-charged moieties include amine groups (e.g., primary, secondary, and tertiary amines), ammonium groups, pyridinium group, guanidine groups, and imidazolium groups. In some embodiments, the charged moieties comprise amine groups. Examples of negatively-charged groups or precursors thereof, include carboxylate groups, sulphonate groups, sulphate groups, phosphonate groups, phosphate groups, hydroxyl groups, and the like. The charge of the charged moiety may vary, in some cases, with the environmental conditions, for example, changes in pH may alter the charge of the moiety, and/or cause the moiety to become charged or uncharged. In general, the charge density of the molecule may be selected as desired. Ionizable lipids can also be the compounds disclosed in International Publication Nos. WO2017/075531, WO2015/199952, WO2013/086354, or WO2013/116126, or selected from formulae CLI-CLXXXXII of U.S. Pat. No. 7,404,969 (each of which is hereby incorporated by reference in its entirety for this purpose).

It should be understood that the terms “charged” or “charged moiety” does not refer to a “partial negative charge” or “partial positive charge” on a molecule. The terms “partial negative charge” and “partial positive charge” are given its ordinary meaning in the art. A “partial negative charge” may result when a functional group comprises a bond that becomes polarized such that electron density is pulled toward one atom of the bond, creating a partial negative charge on the atom. Those of ordinary skill in the art will, in general, recognize bonds that can become polarized in this way.

In some embodiments, the ionizable lipid is an ionizable amino lipid, sometimes referred to in the art as an “ionizable cationic lipid”. In one embodiment, the ionizable amino lipid may have a positively charged hydrophilic head and a hydrophobic tail that are connected via a linker structure. In addition to these, an ionizable lipid may also be a lipid including a cyclic amine group.

The PD-L1 bicyclic peptide mimetic of the present disclosure may be, in some embodiments, formulated into lipid nanoparticles. The lipid nanoparticle may comprise at least one ionizable amino lipid, at least one non-cationic lipid, at least one sterol, and/or at least one polyethylene glycol (PEG)-modified lipid.

In some embodiments, the lipid nanoparticle comprises a molar ratio of 20-60% ionizable amino lipid. For example, the lipid nanoparticle may comprise a molar ratio of 20-50%, 20-40%, 20-30%, 30-60%, 30-50%, 30-40%, 40-60%, 40-50%, or 50-60% ionizable amino lipid. In some embodiments, the lipid nanoparticle comprises a molar ratio of 20%, 30%, 40%, 50, or 60% ionizable amino lipid.

In some embodiments, the lipid nanoparticle comprises a molar ratio of 5-25% non-cationic lipid. For example, the lipid nanoparticle may comprise a molar ratio of 5-20%, 5-15%, 5-10%, 10-25%, 10-20%, 10-25%, 15-25%, 15-20%, or 20-25% non-cationic lipid. In some embodiments, the lipid nanoparticle comprises a molar ratio of 5%, 10%, 15%, 20%, or 25% non-cationic lipid.

In some embodiments, the lipid nanoparticle comprises a molar ratio of 25-55% sterol. For example, the lipid nanoparticle may comprise a molar ratio of 25-50%, 25-45%, 25-40%, 25-35%, 25-30%, 30-55%, 30-50%, 30-45%, 30-40%, 30-35%, 35-55%, 35-50%, 35-45%, 35-40%, 40-55%, 40-50%, 40-45%, 45-55%, 45-50%, or 50-55% sterol. In some embodiments, the lipid nanoparticle comprises a molar ratio of 25%, 30%, 35%, 40%, 45%, 50%, or 55% sterol.

In some embodiments, the lipid nanoparticle comprises a molar ratio of 0.5-15% PEG-modified lipid. For example, the lipid nanoparticle may comprise a molar ratio of 0.5-10%, 0.5-5%, 1-15%, 1-10%, 1-5%, 2-15%, 2-10%, 2-5%, 5-15%, 5-10%, or 10-15%. In some embodiments, the lipid nanoparticle comprises a molar ratio of 0.5%, 1%, 2%, 3%, 4%, 5%, 6%, 7%, 8%, 9%, 10%, 11%, 12%, 13%, 14%, or 15% PEG-modified lipid.

In some embodiments, the lipid nanoparticle comprises a molar ratio of 20-60% ionizable amino lipid, 5-25% non-cationic lipid, 25-55% sterol, and 0.5-15% PEG-modified lipid.

In some specific embodiments the lipid nanoparticle comprises one or more of the lipids POPC, cholesterol, and/or DSPE-PEG. More specifically, the lipid nanoparticle may comprise each of POPC, cholesterol, and DSPE-PEG.

### 3.2 Dosage

It is advantageous to formulate the compositions in dosage unit form for ease of administration and uniformity of dosage. The determination of the novel dosage unit forms of the present invention is dictated by and directly dependent on the unique characteristics of the active material, the particular therapeutic effect to be achieved and the limitations inherent in the art of compounding active materials for the treatment of disease in living subjects having a diseased condition in which bodily health is impaired as herein disclosed in detail.

While the PD-L1 bicyclic peptide mimetic of the invention may be the sole active ingredient administered to the subject, the administration of other cancer therapies concurrently with said PD-L1 bicyclic peptide mimetic is within the scope of the invention. For example, the PD-L1 bicyclic peptide mimetic of Formula I, of SEQ ID NO: 1, or variant described herein may be administered concurrently with one or more cancer therapies, non-limiting examples of which include radiotherapy, surgery, chemotherapy, hormone ablation therapy, pro-apoptosis therapy and immunotherapy The PD-L1 bicyclic peptide mimetic of the invention may be therapeutically used before treatment with the cancer therapy, may be therapeutically used after the cancer therapy or may be therapeutically used together with the cancer therapy.

Suitable radiotherapies include radiation and waves that induce DNA damage, for example, γ-irradiation, X-rays, UV irradiation, microwaves, electronic emissions and radioisotopes. Typically, therapy may be achieved by irradiating the localized tumour site with the above-described forms of radiations. It is most likely that all of these factors cause a broad range of damage to DNA, on the precursors of DNA, on the replication and repair of DNA and on the assembly and maintenance of chromosomes.

The dosage range for X-rays ranges from daily doses of 50-200 roentgens for prolonged periods of time such as 3-4 weeks, to single doses of 2000-6000 roentgens. Dosage ranges for radioisotopes vary widely and depend on the half-life of the isotope, the strength and type of radiation emitted and the uptake by the neoplastic cells. Suitable radiotherapies may include, but are not limited to, conformal external beam radiotherapy (50-100 Gray given as fractions over 4-8 weeks), either single shot or fractionated high dose brachytherapy, permanent interstitial brachytherapy and systemic radioisotopes such as strontium 89. In some embodiments, the radiotherapy may be administered with a radiosensitizing agent. Suitable radiosensitizing agents may include, but are not limited to, efaproxiral, etanidazole, fluosol, misonidazole, nimorazole, temoporfin and tirapazamine.

### 3.3 Combinations

Suitable chemotherapeutic agents may include, but are not limited to, antiproliferative/antineoplastic drugs and combinations thereof including alkylating agents (for example cisplatin, carboplatin, cyclophosphamide, nitrogen mustard, melphalan, chlorambucil, busulphan and nitrosoureas), antimetabolites (for example antifolates such as fluoropyridines like 5-fluorouracil and tegafur, raltitrexed, methotrexate, cytosine arabinoside and hydroxyurea), anti-tumour antibiotics (for example anthracydines like adriamycin, bleomycin, doxorubicin, daunomycin, epirubicin, idarubicin, mitomycin-C, dactinomycin and mithramycin), antimitotic agents (for example Vinca alkaloids like vincristine, vinblastine, vindesine and vinorelbine and taxoids like paditaxel and docetaxel), and topoisomerase inhibitors (for example epipodophyllotoxins like etoposide and teniposide, amsacrine, topotecan and camptothecin); cytostatic agents such as antiestrogens (for example tamoxifen, toremifene, raloxifene, droloxifene and idoxifene), estrogen receptor down regulators (for example fulvestrant), antiandrogens (for example bicalutamide, flutamide, nilutamide and cyproterone acetate), UH antagonists or LHRH agonists (for example goserelin, leuprorelin and buserelin), progestogens (for example megestrol acetate), aromatase inhibitors (for example as anastrozole, letrozole, vorozole and exemestane) and inhibitors of 5a-reductase such as finasteride; agents which inhibit cancer cell invasion (for example metalloproteinase inhibitors like marimastat and inhibitors of urokinase plasminogen activator receptor function); inhibitors of growth factor function, for example such inhibitors include growth factor antibodies, growth factor receptor antibodies (for example the anti-erbb2 antibody trastuzumab [HERCEPTIN™] and the anti-erbbl antibody Cetuximab [C225]), farnesyl transferase inhibitors, MEK inhibitors, tyrosine kinase inhibitors and serine/threonine kinase inhibitors, for example other inhibitors of the epidermal growth factor family (for example other EGFR family tyrosine kinase inhibitors such as N-(3-chloro-4-fluorophenyl)-7-methoxy-6-(3-morpholinopropoxy)quinazolin-4-amine (Gefitinib, AZD1839), N-(3-ethynylphenyl)-6,7-bis(2-methoxyethoxy)quinazolin-4-amine (Erlotinib, OSI-774) and 6-acrylamido-N-(3-chloro-4-fluorophenyl)-7-(3-morpholinopropoxy)quinazolin-4-amine (Cl 1033)), for example inhibitors of the platelet-derived growth factor family and for example inhibitors of the hepatocyte growth factor family; anti-angiogenic agents such as those which inhibit the effects of vascular endothelial growth factor, (for example the anti-vascular endothelial cell growth factor antibody bevacizumab [AVASTIN™], compounds such as those disclosed in International Patent Publication Nos. WO 97/22596, WO 97/30035, WO 97/32856 and WO 98/13354) and compounds that work by other mechanisms (for example linomide, inhibitors of integrin anb3 function and angiostatin); cyclin-dependent kinase inhibitors such as palbocidib, abemacidib, riboddib and alvoddib; vascular damaging agents such as Combretastatin A4 and compounds disclosed in International Patent Publication Nos. WO 99/02166, WO 00/40529, WO 00/41669, WO 01/92224, WO 02/04434 and WO 02/08213; antisense therapies, for example those which are directed to the targets listed above, such as ISIS 2503, an anti-ras antisense; and gene therapy approaches, including for example approaches to replace aberrant genes such as aberrant p53 or aberrant GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy.

Suitable immunotherapy approaches may include, but are not limited to ex vivo and in vivo approaches to increase the immunogenicity of patient tumour cells such as transfection with cytokines including interleukin 2, interleukin 4 or granulocyte-colony stimulating factor; approaches to decrease T-cell anergy; approaches using transfected immune cells such as cytokine-transfected dendritic cells; approaches using cytokine-transfected tumour cell lines; and approaches using anti-idiotypic antibodies. These approaches generally rely on the use of immune effector cells and molecules to target and destroy cancer cells. The immune effector may be, for example, an antibody specific for some marker on the surface of a malignant cell. The antibody alone may serve as an effector of therapy or it may recruit other cells to actually facilitate cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a malignant cell target. Various effector cells include cytotoxic T cells and NK cells.

In some embodiments, the immune effector is a molecule targeting PD-L1, including, but not limited to, an anti-PD-L1 antibody, non-limiting examples of which include atezolizumab, avelumab, durvalumab, BMS-936559, BMS-935559, the antibodies described in International Patent Publication Nos. WO 2013/173223, WO 2013/079174, WO 2010/077634, WO 2011/066389, WO 2010/036959, WO 2007/005874, WO 2004/004771, WO 2006/133396, WO 2013/181634, WO 2012/145493 and Chinese Patent Publication No. CN101104640, clone EH12, and clone 29E.2A3; CA-170; CA-327; BMS-202 (N-[2-[[[2-methoxy-6-[(2-methyl[1,1′-biphenyl]-3-yl)methoxy]-3-pyridinyl]methyl]amino]ethyl]-acetamide); BMS-8 (1-[[3-bromo-4-[(2-methyl[1,1′-biphenyl]-3-yl)methoxy]phenyl]methyl]-2-piperidinecarboxylic acid); the peptides described in Chang et al. (2015) Angew Chem Int Ed Engl, 54(40): 11760-11764, especially (D) PPA-1; AUNP-12; and the peptides described in WO 2014/151634, the entire contents of which are incorporated by reference.

In some embodiments, the immune effector is a molecule targeting PD-1 including, but not limited to, an anti-PD-1 antibody, non-limiting examples of which include nivolumab, pembrolizumab, cemiplimab, tislelizumab (BGB-A317), the antibodies described in WO 2016/106159, WO 2009/114335, WO 2004/004771, WO 2013/173223, WO 2015/112900, WO 2008/156712, WO 2011/159877, WO 2010/036959, WO 2010/089411, WO 2006/133396, WO 2012/145493, WO 2002/078731, anti-mouse PD-1 antibody clone J43, anti-mouse antibody clone RMP1-14, ANB011 (TSR-042), AMP-514 (MEDI0680), WO 2006/121168, WO 2001/014557, WO 2011/110604, WO 2011/110621, WO 2004/072286, WO 2004/056875, WO 2010/036959, WO 2010/029434, and WO 2013/02209; AMP-224; the compounds described in WO 2011/082400; the molecules and antibodies described in U.S. Pat. No. 6,808,710; the molecules and antibodies described in WO 2013/019906; the molecules described in WO 2003/011911; and the compounds described in WO 2013/132317, the entire contents of which are incorporated by reference.

In some embodiments, the immune effector is a molecule targeting PD-L2 including, but not limited to, an anti-PD-L2 antibody, non-limiting examples of which include the antibodies described in International Patent Publication No. WO 2010/036959, the entire content of which is incorporated by reference; and rHigM12B7.

In some embodiments, the immune effector is a molecule targeting CTLA-4 including, but not limited to, an anti-CTLA-4 antibody such as ipilimumab, tremelimumab, the antibodies described in WO 00/37504, WO 01/14424, US 2003/0086930; and the compounds described in WO 2006/056464, the entire contents of which are incorporated by reference.

Examples of other cancer therapies include phytotherapy, cryotherapy, toxin therapy or pro-apoptosis therapy. A person skilled in the art would appreciate that this list is not exhaustive of the types of treatment modalities available for cancer and other hyperplastic lesions.

It is well known that chemotherapy and radiation therapy target rapidly dividing cells and/or disrupt the cell cycle or cell division. These treatments are offered as part of treating several forms of cancer, aiming either at slowing their progression or reversing the symptoms of disease by means of a curative treatment. However, these cancer treatments may lead to an immunocompromised state and ensuing pathogenic infections and, thus, the present invention also extends to combination therapies, which employ a PD-L1 bicyclic peptide mimetic of Formula I, SEQ ID NO: 1, or variant described herein, a cancer therapy and an anti-infective agent that is effective against an infection that develops or that has an increased risk of developing from an immunocompromised condition resulting from the cancer therapy. The anti-infective drug is suitably selected from antimicrobials, which may include, but are not limited to, compounds that kill or inhibit the growth of microorganisms such as viruses, bacteria, yeast, fungi, protozoa, etc. and, thus, include antibiotics, amebicides, antifungals, antiprotozoals, antimalarials, antituberculotics and antivirals. Anti-infective drugs also include within their scope anthelmintics and nematocides. Illustrative antibiotics include quinolones (e.g., amifloxacin, cinoxacin, ciprofloxacin, enoxacin, fleroxacin, flumequine, lomefloxacin, nalidixic acid, norfloxacin, ofloxacin, levofloxacin, lomefloxacin, oxolinic acid, pefloxacin, rosoxacin, temafloxacin, tosufloxacin, sparfloxacin, dinafloxacin, gatifloxacin, moxifloxacin; gemifloxacin; and garenoxacin), tetracyclines, glycylcyclines and oxazolidinones (e.g., chlortetracycline, demedocydine, doxycycline, lymecycline, methacycline, minocycline, oxytetracycline, tetracycline, tigecydine; linezolide, eperezolid), glycopeptides, aminoglycosides (e.g., amikacin, arbekadn, butirosin, dibekadn, fortimicins, gentamicin, kanamydn, menomydn, netilmicin, ribostamydn, sisomicin, spectinomydn, streptomycin, tobramycin), β-lactams (e.g., imipenem, meropenem, biapenem, cefaclor, cefadroxil, cefamandole, cefatrizine, cefazedone, cefazolin, cefixime, cefmenoxime, cefodizime, cefonidd, cefoperazone, ceforanide, cefotaxime, cefotiam, cefpimizole, cefpiramide, cefpodoxime, cefsulodin, ceftazidime, cefteram, ceftezole, ceftibuten, ceftizoxime, ceftriaxone, cefuroxime, cefuzonam, cephacetrile, cephalexin, cephaloglydn, cephaloridine, cephalothin, cephapirin, cephradine, cefinetazole, cefoxitin, cefotetan, azthreonam, carumonam, flomoxef, moxalactam, amdinocilin, amoxicillin, ampicillin, azlocillin, carbenicillin, benzylpenicillin, carfecilin, doxacillin, didoxacillin, methidllin, mezlocillin, nacillin, oxacillin, penicillin G, piperacillin, sulbenicillin, temocillin, ticardllin, cefditoren, SC004, KY-020, cefdinir, ceftibuten, FK-312, S-1090, CP-0467, BK-218, FK-037, DQ-2556, FK-518, cefozopran, ME1228, KP-736, CP-6232, Ro 09-1227, OPC-20000, LY206763), rifamycins, macrolides (e.g., azithromycin, clarithromycin, erythromycin, oleandomycin, rokitamycin, rosaramicin, roxithromycin, troleandomycin), ketolides (e.g., telithromycin, cethromycin), coumermycins, lincosamides (e.g., clindamycin, lincomycin) and chloramphenicol.

Illustrative antivirals include abacavir sulfate, acyclovir sodium, amantadine hydrochloride, amprenavir, cidofovir, delavirdine mesylate, didanosine, efavirenz, famciclovir, fomivirsen sodium, foscarnet sodium, ganciclovir, indinavir sulfate, lamivudine lamivudine/zidovudine, nelfinavir mesylate, nevirapine, oseltamivir phosphate, ribavirin, rimantadine hydrochloride, ritonavir, saquinavir, saquinavir mesylate, stavudine, valacydovir hydrochloride, zalcitabine, zanamivir and zidovudine.

Suitable amebicides or antiprotozoals include, but are not limited to, atovaquone, chloroquine hydrochloride, chloroquine phosphate, metronidazole, metronidazole hydrochloride and pentamidine isethionate. Anthelmintics can be at least one selected from mebendazole, pyrantel pamoate, albendazole, ivermectin and thiabendazole. Illustrative antifungals can be selected from amphotericin B, amphotericin B cholesteryl sulfate complex, amphotericin B lipid complex, amphotericin B liposomal, fluconazole, flucytosine, griseofulvin microsize, griseofulvin ultramicrosize, itraconazole, ketoconazole, nystatin and terbinafine hydrochloride. Suitable antimalarials include, but are not limited to, chloroquine hydrochloride, chloroquine phosphate, doxycycline, hydroxychloroquine sulfate, mefloquine hydrochloride, primaquine phosphate, pyrimethamine and pyrimethamine with sulfadoxine. Antituberculotics include but are not restricted to clofazimine, cycloserine, dapsone, ethambutol hydrochloride, isoniazid, pyrazinamide, rifabutin, rifampin, rifapentine, and streptomycin sulfate.

As previously described, the PD-L1 bicyclic peptide mimetic may be compounded for convenient and effective administration in effective amounts with a suitable pharmaceutically acceptable carrier in dosage unit form. In some embodiments, a unit dosage form may comprise the active peptide of the invention in amount in the range of from about 0.25 pg to about 2000 mg. The active peptide of the invention may be present in an amount of from about 0.25 pg to about 2000 mg/mL of carrier. In embodiments where the pharmaceutical composition comprises one or more additional active ingredients, the dosages are determined by reference to the usual dose and manner of administration of the said ingredients.

### 4. METHODS

The present inventors have determined that PD-L1 bicyclic peptide mimetics comprising an amino acid sequence corresponding to Formula I inhibit or reduce nuclear localization of PD-L1. It was previously known that acetylation of an acetylation site of PD-L1 increases its nuclear localization in a cell. However, in particular, the present inventors have found that a PD-L1 bicyclic peptide mimetic corresponding to an acetylation site in PD-L1, reduces or inhibits nuclear localization of PD-L1. The present inventors have conceived that the PD-L1 bicyclic peptide mimetics of the invention may be useful in methods for altering at least one of formation, proliferation, maintenance, EMT, MET or viability of a PD-L1 overexpressing cell and are useful for the treatment or prevention of a condition involving PD-L1 nuclear localization in a subject, such as a cancer.

Without wishing to be bound by theory, the inventors have determined that acetylation of PD-L1, increases nuclear localization of the polypeptide and, thus, it is proposed that inhibition of acetylation of the PD-L1 will also inhibit or reduce nuclear localization of PD-L1. Furthermore, it is proposed that a PD-L1 bicyclic peptide mimetic which corresponds to an acetylation site will competitively inhibit acetylation of the nuclear localizable polypeptide and, thus, decrease nuclear localization of PD-L1.

More specifically, the present inventors identified that the PD-L1 bicyclic peptide mimetics of the present invention inhibited or otherwise disrupted the interaction between a PD-L1 polypeptide and importin. Importin is a nuclear transport molecule that binds to PD-L1 in order to transport the complex into the cell nucleus. The PD-L1 bicyclic peptide mimetics of this invention are specific inhibitors of the PD-L1-Impotin complex. In some particularly preferred embodiments, the PD-L1 bicyclic peptide mimetics do not significantly inhibit or disrupt the interactions between importin and any other polypeptide. In some embodiments, the importin is an importin α (e.g., importin α1).

Accordingly, in another aspect of the invention, there is provided a method of inhibiting or reducing the nuclear localization of PD-L1, the method comprising contacting the cell with a PD-L1 bicyclic peptide mimetic comprising, consisting or consisting essentially of an amino acid sequence corresponding Formula I (e.g., the amino acid sequence set forth in SEQ ID NO: 1).

The PD-L1 bicyclic peptide mimetic includes an amino acid that corresponding to an acetylation site of the native wild-type PD-L1 amino acid sequence. Acetylation of PD-L1 is generally performed by an acetyltransferase; especially a histone acetyltransferase, including, but not limited to, GCN5, Hatl, ATF-2, Tip60, MOZ, MORF, HB01, p300, CBP, SRC-1, ACTR, TIF-2, SRC-3, TAF1, TFIIIC and/or CLOCK; especially p300.

In particular embodiments, the amino acid sequence of the PD-L1 bicyclic peptide mimetic corresponds to a lysine acetylation site (i.e., an acetylation site wherein a lysine residue is acetylated); especially a PD-L1 lysine acetylation site; most especially residues 255 to 271 of PD- L1.

The amino acid sequence of PD-L1 (Uniprot Accession No. Q9NZQ7) is presented in SEQ ID NO: 75. The amino acid sequence corresponding to residues 255 to 271 of PD-L1 comprises a potential acetylation site, wherein the E-amino group on lysine 263 is acetylated. Residues 255 to 271 are underlined in the sequence below.

[SEQ ID NO: 75]
MRIFAVFIFMTYWHLLNAFTVTVPKDLYVVEYGSNMTIECKFPVEKQLD
LAALIVYWEMEDKNIIQFVHGEEDLKVQHSSYRQRARLLKDQLSLGNAA
LQITDVKLQDAGVYRCMISYGGADYKRITVKVNAPYNKINQRILVVDPV
TSEHELTCQAEGYPKAEVIWTSSDHQVLSGKTTTTNSKREEKLFNVTST
LRINTTTNEIFYCTFRRLDPEENHTAELVIPELPLAHPPNERTHLVILG
AILLCLGVALTFIFRLRKGRMMDVKKCGIQDTNSKKQSDTHLEET.

In some embodiments, the PD-L1 bicyclic peptide mimetic is an isolated or purified PD-L1 bicyclic peptide mimetic represented by Formula I; particularly the PD-L1 bicyclic peptide mimetic of SEQ ID NO: 1, or variant PD-L1 bicyclic peptide mimetic described herein.

In another aspect of the invention, there is provided a use of the isolated or purified PD-L1 bicyclic peptide mimetic of the invention, particularly the PD-L1 bicyclic peptide mimetic of Formula I, SEQ ID NO: 1, or variant PD-L1 bicyclic peptide mimetic described herein, for therapy or in the manufacture of a medicament for therapy. The invention also provides an isolated or purified PD-L1 bicyclic peptide mimetic of the invention, particularly the PD-L1 bicyclic peptide mimetic of Formula I, SEQ ID NO: 1, or variant PD-L1 bicyclic peptide mimetic described herein, for use in therapy.

The present invention also provides a method of inhibiting or reducing nuclear localization of PD-L1 in a PD-L1-overexpressing cell, comprising contacting the cell with a PD-L1 bicyclic peptide mimetic comprising, consisting or consisting essentially of an amino acid sequence corresponding to an acetylation site. The present invention also contemplates the use of a PD-L1 bicyclic peptide mimetic comprising, consisting or consisting essentially of an amino acid sequence corresponding to an acetylation site for inhibiting or reducing nuclear localization of PD-L1 in a PD-L1-overexpressing cell; a PD-L1 bicyclic peptide mimetic comprising, consisting or consisting essentially of an amino acid sequence corresponding to an acetylation site for use in inhibiting or reducing the nuclear localization of PD-L1 in a PD-L1-overexpressing cell; and in the manufacture of a medicament for such use.

In yet another aspect of the invention, there is provided a method of altering at least one of (i) formation; (ii) proliferation; (ill) maintenance; (iv) EMT; (v) MET; or (vi) viability of a PD-L1-overexpressing cell, comprising contacting said cell with a formation-, proliferation-, maintenance-, EMT-, MET-or viability-modulating amount of a PD-L1 bicyclic peptide mimetic comprising, consisting or consisting essentially of an amino acid sequence corresponding to an acetylation site. The invention also contemplates a use of a PD-L1 bicyclic peptide mimetic comprising, consisting or consisting essentially of an amino acid sequence corresponding to an acetylation site for altering at least one of (i) formation; (ii) proliferation; (ill) maintenance; (iv) EMT; (v) MET; or (vi) viability of a PD-L1-overexpressing cell. The invention also extends to a PD-L1 bicyclic peptide mimetic comprising, consisting or consisting essentially of an amino acid sequence corresponding to an acetylation site for use in altering at least one of (i) formation; (ii) proliferation; (iii) maintenance; (iv) EMT; (v) MET; or (vi) viability of a PD-L1-overexpressing cell; and in the manufacture of a medicament for this use.

In some embodiments of any one of the above aspects, the PD-L1-overexpressing cell is a cancer stem cell or a non-cancer stem cell tumour cell, especially a cancer stem cell tumour cell.

In some embodiments, the PD-L1 bicyclic peptide mimetic results in a reduction, impairment, abrogation, inhibition or prevention of the (i) formation; (ii) proliferation; (iii) maintenance; (iv) EMT or (vi) viability of a PD-L1-overexpressing cell; and/or in the enhancement of (v) MET of a PD-L1-overexpressing cell.

Suitable embodiments of the PD-L1 bicyclic peptide mimetic are as described herein.

PD-L1 bicyclic peptide mimetics comprising, consisting or consisting essentially of an amino acid sequence corresponding to an acetylation site as described herein, especially the PD-L1 bicyclic peptide mimetics of Formula I, SEQ ID NO: 1, or variant PD-L1 bicyclic peptide mimetic, are useful for the inhibition of nuclear localization of PD-L1. Accordingly, the present inventors have conceived that the PD-L1 bicyclic peptide mimetics are useful for treating or preventing a cancer in a subject. Thus, in another aspect, there is provided a method for treating or preventing a cancer in a subject wherein the cancer comprises at least one PD-L1-overexpressing cell, comprising administering to the subject a PD-L1 bicyclic peptide mimetic comprising, consisting or consisting essentially of an amino acid sequence corresponding to an acetylation site. The present invention also extends to a use of a PD-L1 bicyclic peptide mimetic comprising, consisting or consisting essentially of an amino acid sequence corresponding to an acetylation site for treating or preventing a cancer in a subject wherein the cancer comprises at least one PD-L1-overexpressing cell; and in the manufacture of a medicament for this purpose. A PD-L1 bicyclic peptide mimetic comprising, consisting or consisting essentially of an amino acid sequence corresponding to an acetylation site for use in treating or preventing a cancer in a subject wherein the cancer comprises at least one PD-L1-overexpressing cell is also contemplated.

The cancer may be any cancer involving overexpression of PD-L1. Suitable cancers may include, but are not limited to breast, prostate, lung, bladder, pancreatic, colon, liver, ovarian, kidney or brain cancer, or melanoma or retinoblastoma; especially breast cancer, lung cancer or melanoma; most especially breast cancer or melanoma; more especially breast cancer.

In some embodiments, the PD-L1 bicyclic peptide mimetics comprising, consisting or consisting essentially of an amino acid sequence corresponding to an acetylation site as described herein are useful for treating, preventing and/or relieving the symptoms of a malignancy, particularly a metastatic cancer. In preferred embodiments, the PD-L1 bicyclic peptide mimetics are used for treating, preventing and/or relieving the symptoms of a metastatic cancer. Suitable types of metastatic cancer include, but are not limited to, metastatic breast, prostate, lung, bladder, pancreatic, colon, liver, ovarian, kidney or brain cancer, or melanoma or retinoblastoma. In some embodiments, the brain cancer is a glioma. In preferred embodiments, the metastatic cancer is metastatic breast cancer, lung cancer or melanoma; especially metastatic breast cancer or melanoma; most especially metastatic breast cancer.

The PD-L1 bicyclic peptide mimetics are useful in methods involving PD-L1-overexpressing cells. In particular embodiments, the PD-L1-overexpressing cell is selected from a breast, prostate, testicular, lung, bladder, pancreatic, colon, melanoma, leukemia, retinoblastoma, liver, ovary, kidney or brain cell; especially a breast, lung or melanoma cell; most especially a breast or melanoma cell; more especially a breast cell. In preferred embodiments, the PD-L1-overexpressing cell is a breast epithelial cell, especially a breast ductal epithelial cell.

In some embodiments, the PD-L1-overexpressing cell is a cancer stem cell or a non-cancer stem cell tumour cell; especially a cancer stem cell tumour cell; most especially a breast cancer stem cell tumour cell. In some embodiments, the cancer stem cell tumour cell expresses CD24 and CD44, particularly CD44^{high}, CD24^{low}.

In some embodiments, the methods further comprise detecting overexpression of a PDL1 gene in a tumour sample obtained from the subject, wherein the tumour sample comprises the cancer stem cell tumour cells and optionally the non-cancer stem cell tumour cells, prior to administering the PD-L1 bicyclic peptide mimetic to the subject.

The PD-L1 bicyclic peptide mimetics comprising, consisting or consisting essentially of an amino acid sequence corresponding to an acetylation site as described herein are suitable for treating an individual who has been diagnosed with a cancer, who is suspected of having a cancer, who is known to be susceptible and who is considered likely to develop a cancer, or who is considered to develop a recurrence of a previously treated cancer. The cancer may be hormone receptor negative. In some embodiments, the cancer is hormone receptor negative and is, thus, resistant to hormone or endocrine therapy. In some embodiments where the cancer is breast cancer, the breast cancer is hormone receptor negative. In some embodiments, the breast cancer is estrogen receptor negative and/or progesterone receptor negative.

There are numerous conditions involving PD-L1-overexpression in which the PD-L1 bicyclic peptide mimetics comprising, consisting or consisting essentially of an amino acid sequence corresponding to an acetylation site as described herein may be useful. Accordingly, in another aspect of the invention, there is provided a method of treating or preventing a condition in a subject in respect of which inhibition or reduction of nuclear localization of PD-L1 is associated with effective treatment, comprising administering to the subject a PD-L1 bicyclic peptide mimetic comprising, consisting or consisting essentially of an amino acid sequence corresponding to an acetylation site. The invention also provides a use of a PD-L1 bicyclic peptide mimetic comprising, consisting or consisting essentially of an amino acid sequence corresponding to an acetylation site for treating or preventing a condition in a subject in respect of which inhibition or reduction of nuclear localization of PD-L1 is associated with effective treatment; a PD-L1 bicyclic peptide mimetic comprising, consisting or consisting essentially of an amino acid sequence corresponding to an acetylation site for use in treating or preventing a condition in a subject in respect of which inhibition or reduction of nuclear localization of PD-L1 is associated with effective treatment; and a use of a PD-L1 bicyclic peptide mimetic comprising, consisting or consisting essentially of an amino acid sequence corresponding to an acetylation site in the manufacture of a medicament for this purpose.

Non-limiting examples of conditions involving PD-L1-overexpression include cancer, infection, autoimmune disorders and respiratory disorders.

In some embodiments, the infection is a pathogenic infection. The infection may be selected from, but is not limited to, a viral, bacterial, yeast, fungal, helminth or protozoan infection. Viral infections contemplated by the present invention include, but are not restricted to, infections caused by HIV, hepatitis, influenza virus, Japanese encephalitis virus, Epstein-Barr virus, herpes simplex virus, filovirus, human papillomavirus, human T-cell lymphotropic virus, human retrovirus, cytomegalovirus, varicella-zoster virus, poliovirus, measles virus, rubella virus, mumps virus, adenovirus, enterovirus, rhinovirus, ebola virus, West Nile virus and respiratory syncytial virus; especially infections caused by HIV, hepatitis, influenza virus, Japanese encephalitis virus, Epstein-Barr virus and respiratory syncytial virus. Bacterial infections include, but are not restricted to, those caused by Neisseria species, Meningococcal species, Haemophilus species, Salmonella species, Streptococcal species, Legionella species, Mycoplasma species, Bacillus species, Staphylococcus species, Chlamydia species, Actinomyces species, Anabaena species, Bacteroides species, Bdellovibrio species, Bordetella species, Borrella species, Campylobacter species, Caulobacter species, Chlrorbium species, Chromatium species, Chlostridium species, Corynebacterlum species, Cytophaga species, Deinococcus species, Escherichia species, Francisella species, Helicobacter species, Haemophilus species, Hyphomicrobium species, Leptospira species, Usteria species, Micrococcus species, Myxococcus species, Nitrobacter species, Osclllatoila species, Prochloron species, Proteus species, Pseudomonas species, Rhodospirillum species, Rickettsia species, Shigella species, Spirillum species, Spirochaeta species, Streptomyces species, Thiobacillus species, Treponema species, Vibrio species, Yersinia species, Nocardia species and Mycobacterium species; especially infections caused by Neisseria species, Meningococcal species, Haemophilus species, Salmonella species, Streptococcal species, Legionella species, and Mycobacterium species. Protozoan infections encompassed by the invention include, but are not restricted to, those caused by Plasmodium species, Leishmania species, Trypanosoma species, Toxoplasma species, Entamoeba species and Giardia species. Helminth infections may include, but are not limited to, infections caused by Schistosoma species. Fungal infections contemplated by the present invention include, but are not limited to, infections caused by Histoplasma species and Candida species.

Suitable autoimmune disorders include, but are not limited to, autoimmune rheumatologic disorders (such as, for example, rheumatoid arthritis, Sjogren's syndrome, scleroderma, lupus such as systemic lupus erythematosus (SLE) and lupus nephritis, polymyositis-dermatomyositis, cryoglobulinemia, anti-phospholipid antibody syndrome and psoriatic arthritis), autoimmune gastrointestinal and liver disorders (such as, for example, inflammatory bowel diseases e.g., ulcerative colitis and Crohn's disease, autoimmune gastritis and pernicious anemia, autoimmune hepatitis, primary biliary cirrhosis, primary sclerosing cholangitis and celiac disease), vasculitis (such as, for example, anti-neutrophil cytoplasmic antibody (ANCA)-negative vasculitis and ANCA-associated vasculitis, including Churg-Strauss vasculitis, Wegener's granulomatosis and microscopic polyangiitis), autoimmune neurological disorders (such as, for example, multiple sclerosis, opsoclonus myoclonus syndrome, myasthenia gravis, neuromyelitis optica, Parkinson's disease, Alzheimer's disease and autoimmune polyneuropathies), renal disorders (such as, for example, glomerulonephritis, Goodpasture's syndrome, and Berger's disease), autoimmune dermatologic disorders (such as, for example, psoriasis, urticaria, hives, pemphigus vulgaris, bullous pemphigoid and cutaneous lupus erythematosus), hematologic disorders (such as, for example, thrombocytopenic purpura, thrombotic thrombocytopenic purpura, post-transfusion purpura, and autoimmune hemolytic anemia), atherosclerosis, uveitis, autoimmune hearing diseases (such as, for example, inner ear disease and hearing loss), Behcet's disease, Raynaud's syndrome, organ transplant, and autoimmune endocrine disorders (such as, for example, diabetic-related autoimmune diseases such as type I diabetes mellitis, Addison's disease and autoimmune thyroid disease (e.g., Graves' disease and thyroiditis)).

Suitable respiratory disorders include, but are not limited to, chronic obstructive pulmonary disease (CORD) or asthma especially allergic asthma.

In some embodiments, the methods further comprise detecting overexpression of a PD-L1 gene in a tumour sample obtained from the subject, wherein the tumour sample comprises the cancer stem cell tumour cells and optionally the non-cancer stem cell tumour cells, prior to administering the PD-L1 bicyclic peptide mimetic of the invention to the subject.

In particular embodiments, any one of the methods described above involve the administration of one or more further active agents as described in Section 3.3 supra, such as an additional cancer therapy and/or an anti-infective agent, especially an additional cancer therapy.

The PD-L1 bicyclic peptide mimetics of the invention, especially a PD-L1 bicyclic peptide mimetic of Formula 1, SEQ ID NO: 1, or variant PD-L1 bicyclic peptide mimetic as described herein, are useful for inhibiting or reducing the acetylation of PD-L1. In some embodiments, the acetylation is catalyzed by an acetyltransferase; especially a histone acetyltransferase. In some embodiments, the histone acetyltransferase is GCNS, Hatl, ATF-2, Tip60, MOZ, MORF, HBO1, p300, CBP, SRC-1, ACTR, TIF-2, SRC-3, TAF1, TFIIIC and/or CLOCK; especially p300.

Thus, in a further aspect of the invention, there is provided a method of inhibiting the catalytic activity of an acetyltransferase in a subject, comprising administering a PD-L1 bicyclic peptide mimetic comprising, consisting or consisting essentially of an amino acid sequence corresponding to an acetylation site, embodiments of which are described herein. The invention also extends to a use of a PD-L1 bicyclic peptide mimetic described herein for inhibiting the catalytic activity of an acetyltransferase in a subject, and a PD-L1 bicyclic peptide mimetic described herein for use in inhibiting the catalytic activity of an acetyltransferase in a subject. In preferred embodiments, the acetyltransferase is a histone acetyltransferase, embodiments of which are described above.

The present invention also contemplates a method of producing a PD-L1 bicyclic peptide mimetic that inhibits or reduces nuclear localization of a PD-L1, wherein acetylation of an acetylation site of PD-L1 increases its nuclear localization in a cell, the method comprising:
a) contacting a cell with a PD-L1 bicyclic peptide mimetic comprising, consisting or consisting essentially of an amino acid sequence corresponding to an acetylation site; and b) detecting a reduction in or inhibition of the nuclear localization of the nuclear localizable polypeptide in the cell relative to a normal or reference level of nuclear localization in the absence of the PD-L1 bicyclic peptide mimetic.

The present invention also contemplates a method of producing a PD-L1 bicyclic peptide mimetic that inhibits or reduces nuclear localization of a PD-L1, wherein the binding of PD-L1 with importin (e.g., importin α) increases its nuclear localisation in a cell, the method comprising:
a) contacting a cell with a PD-L1 bicyclic peptide mimetic comprising, consisting or consisting essentially of an amino acid sequence corresponding to a PD-L1 amino acid sequence; and
b) detecting a reduction in or inhibition of binding of PD-L1 to importin (e.g., importin α) in the cell relative to a normal or reference level of binding of PD-L1 to importin in the absence of the PD-L1 bicyclic peptide mimetic.

In another aspect, the present invention provides a method of producing a PD-L1 bicyclic peptide mimetic that inhibits or reduces nuclear localization of PD-L1, wherein acetylation of an acetylation site of PD-L1 increases its nuclear localization in a cell, the method comprising:
a) contacting a cell with a PD-L1 bicyclic peptide mimetic comprising, consisting or consisting essentially of an amino acid sequence set forth in Formula I; and
b) detecting a reduction in or inhibition of the nuclear localization of the nuclear localizable polypeptide in the cell relative to a normal or reference level of nuclear localization in the absence of the PD-L1 bicyclic peptide mimetic.

The present invention also contemplates a method of producing a PD-L1 bicyclic peptide mimetic that inhibits or reduces nuclear localization of a PD-L1, wherein the binding of PD-L1 with importin (e.g., importin a) increases its nuclear localisation in a cell, the method comprising:
a) contacting a cell with a PD-L1 bicyclic peptide mimetic comprising, consisting or consisting essentially of an amino acid sequence in accordance with Formula I; and
b) detecting a reduction in or inhibition of binding of PD-L1 to importin (e.g., importin a) in the cell relative to a normal or reference level of binding of PD-L1 to importin in the absence of the PD-L1 bicyclic peptide mimetic.

In some embodiments, the PD-L1 bicyclic peptide mimetic is distinguished from a native wild-type PD-L1 sequence (e.g., the native PD-L1 NLS) at least by the addition of three cysteine residues.

A reduction in or inhibition of the nuclear localization of PD-L1 may be determined using standard techniques in the art, non-limiting examples of which include immunofluorescence, immunohistochemistry staining, chromatin immunoprecipitation (ChIP), ChIP-seq, chromatin accessibility assays such as DNase-seq, FAIRE-seq and ATAC-seq assays, such as that described in Satelli et al. (2016) Sci Rep, 6:28910; Bajetto, et al. (2000) Brain Research Protocols, 5(3) : 273-281; and Sung, et al. (2014) BMC Cancer, 14:951, the entire contents of which are incorporated by reference.

In yet another aspect, there is provided a method of producing a PD-L1 bicyclic peptide mimetic that inhibits or reduces at least one of formation, proliferation, viability or EMT of a cancer cell (e.g., a cancer stem cell), the method comprising:
a) contacting a cancer stem cell with a PD-L1 bicyclic peptide mimetic comprising, consisting or consisting essentially of an amino acid sequence set forth in Formula I; and
b) detecting a reduction in or inhibition of the formation, proliferation or EMT of the cancer stem cell relative to a normal or reference level of formation, proliferation, viability or EMT of the cell in the absence of the PD-L1 bicyclic peptide mimetic.

In some embodiments, the PD-L1 bicyclic peptide mimetic is distinguished from PD-L1 (e.g., the PD-L1 NLS) at least by the addition of three cysteine residues.

The amino acid sequence of the PD-L1 bicyclic peptide mimetic may correspond to a natural, designed or synthetic acetylation site. In some embodiments, the acetylation site is a site of PD-L1. Suitable acetylation sites are as previously described herein. In other embodiments, the amino acid sequence corresponding to an acetylation site is other than an amino acid sequence of PD-L1.

A PD-L1 bicyclic peptide mimetic with an amino acid sequence corresponding to a designed acetylation site may be identified using medicinal chemistry techniques standard in the art.

An acetylation site of a polypeptide may be identified using computational methods such as that described in Hake and Janzen (2013) Protein Acetylation: Methods and

Protocols, Methods in Molecular Biology, vol. 981; Li, et al. (2014) Srf Rep, 4:5765; Hou, et al. (2014) PLoS One, 9(2):e89575; and Wuyun, et al. (2016) PLoS One, 11(5):e0155370 (the entire contents of which are incorporated by reference); or can be determined experimentally using, for example, mutagenesis of predicted residues in combination with detection of levels of acetylation using, for example, an antibody directed to an acetylated amino acid residue such as an acetylated lysine. The involvement of surrounding and/or proximal residues may be determined using medicinal chemistry techniques standard in the art.

A skilled person would be well aware of suitable assays used to evaluate the nuclear localization of a polypeptide, such as PD-L1, and to identify PD-L1 bicyclic peptide mimetics that inhibit or reduce the nuclear localization of a polypeptide. Screening for active agents according to the invention can be achieved by any suitable method. For example, the method may include contacting a cell expressing a polynucleotide corresponding to a gene that encodes the polypeptide of interest, such as PD-L1, with an agent suspected of having the inhibitory activity and screening for the inhibition or reduction of the level of the polypeptide of interest in the nucleus of the cell.

Alternatively, the inhibition of the functional activity of the polypeptide of interest or the lowering of the level of a transcript encoded by the polynucleotide, or the inhibition of the activity or expression of a downstream cellular target of the polypeptide or of the transcript, may be screened wherein the activity is related to nuclear localization of PD-L1. Detecting such inhibition may be achieved utilizing techniques including, but not limited to, ELISA, immunofluorescence, Western blots, immunoprecipitation, immunostaining, slot or dot blot assays, scintillation proximity assays, fluorescent immunoassays using antigen-binding molecule conjugates or antigen conjugates of fluorescent substances such as fluorescein or rhodamine, RIA, Ouchterlony double diffusion analysis, immunoassays employing an avidin-biotin or a streptavidin-biotin detection system, nucleic acid detection assays including reverse transcriptase polymerase chain reaction (RT-PCR), cell proliferation assays such as a WST-1 proliferation assay and immunoblot analysis of cells treated with PD-L1 Half-Way ChIP. The acetylation of a polypeptide may be determined using an antibody directed to the acetylated polypeptide, such as an antibody directed to an acetylated lysine residue.

It will be understood that a polynucleotide from which PD-L1 is regulated or expressed may be naturally occurring in the cell which is the subject of testing or it may have been introduced into the host cell for the purposes of testing.

The inhibition of the catalytic activity of an acetylase may be determined using techniques standard in the art. For example, the inhibition of an acetyltransferase may be assessed using a fluorescence assay such as the acetyltransferase activity assay kit from Abeam (Catalogue number ab204536), the p300 fluorogenic assay kit from BPS Bioscience (Catalogue number 50092), or the p300 inhibitor screening assay kit (fluorometric) from Abeam (Catalogue number ab196996); a colorimetric assay such as the histone acetyltransferase activity assay kit from Abeam (Catalogue number ab65352); or a chemiluminescence assay such as the p300 chemiluminescent assay kit from BPS Bioscience (Catalogue number 50077).

These methods provide a mechanism for performing high throughput screening of putative modulatory agents such as proteinaceous agents comprising synthetic, combinatorial, chemical and natural libraries.

Active molecules may be further tested in the animal models to identify those molecules having the most potent in vivo effects. These molecules may serve as lead molecules for the further development of pharmaceuticals by, for example, subjecting the compounds to sequential modifications, molecular modeling and other routine procedures employed in rational drug design.

### 5. KITS

In other embodiments of the invention, therapeutic kits are provided comprising a PD-L1 bicycle peptide mimetic and an anticancer spray. In some embodiments, the therapeutic kits further comprise a package insert comprising instructional material for administering concurrently the PD-L1 bicycle peptide mimetic and anti-cancer agent to treat a T cell dysfunctional disorder, or to enhance immune function (e.g., immune effector function, T cell function etc.) in an individual having cancer, or to treat or delay cancer progression, or to treat infection in an individual. In some embodiments, the anti-cancer agent comprises a chemotherapeutic agent (e.g., an agent that targets rapidly dividing cells and/or disrupt the cell cycle or cell division, representative examples of which include cytotoxic compounds such as a taxane).

In some embodiments, the LSD inhibitor, PD-1 binding antagonist and optionally chemotherapeutic agents are in the same container or separate containers. Suitable containers include, for example, bottles, vials, bags and syringes. The container may be formed from a variety of materials such as glass, plastic (such as polyvinyl chloride or polyolefin), or metal alloy (such as stainless steel or hastelloy). In some embodiments, the container holds the formulation and the label on, or associated with, the container may indicate directions for use. The kits may further include other materials desirable from a commercial and user standpoint, including other buffers, diluents, filters, needles, syringes, and package inserts with instructional material for use. In some embodiments, the kits further include one or more of other agents (e.g., a chemotherapeutic agent, and anti-neoplastic agent). Suitable containers for the one or more agent include, for example, bottles, vials, bags and syringes.

In other embodiments of the invention, diagnostic kits are provided for determining expression of biomarkers, including the T-cell function biomarkers disclosed herein, which include reagents that allow detection and/or quantification of the biomarkers. Such reagents include, for example, compounds or materials, or sets of compounds or materials, which allow quantification of the biomarkers. In specific embodiments, the compounds, materials or sets of compounds or materials permit determining the expression level of a gene (e.g., T-cell function biomarker gene), including without limitation the extraction of RNA material, the determination of the level of a corresponding RNA, etc., primers for the synthesis of a corresponding cDNA, primers for amplification of DNA, and/or probes capable of specifically hybridizing with the RNAs (or the corresponding cDNAs) encoded by the genes, TaqMan probes, proximity assay probes, ligases, antibodies etc.

The kits may also optionally include appropriate reagents for detection of labels, positive and negative controls, washing solutions, blotting membranes, microtiter plates, dilution buffers and the like. For example, a nucleic acid-based detection kit may include (i) a T-cell function biomarker polynucleotide (which may be used as a positive control), (ii) a primer or probe that specifically hybridizes to a T-cell function biomarker polynucleotide. Also included may be enzymes suitable for amplifying nucleic acids including various polymerases (reverse transcriptase, Taq, Sequenase™, DNA ligase etc. depending on the nucleic acid amplification technique employed), deoxynucleotides and buffers to provide the necessary reaction mixture for amplification. Such kits also generally will comprise, in suitable means, distinct containers for each individual reagent and enzyme as well as for each primer or probe. Alternatively, a protein-based detection kit may include (i) a T-cell function biomarker polypeptide (which may be used as a positive control), (ii) an antibody that binds specifically to a T-cell function biomarker polypeptide. The kit can also feature various devices (e.g., one or more) and reagents (e.g., one or more) for performing one of the assays described herein; and/or printed instructional material for using the kit to quantify the expression of a T-cell function biomarker gene. The reagents described herein, which may be optionally associated with detectable labels, can be presented in the format of a microfluidics card, a chip or chamber, a microarray or a kit adapted for use with the assays described in the examples or below, e.g., RT-PCR or Q PCR techniques described herein.

Materials suitable for packing the components of the diagnostic kits may include crystal, plastic (polyethylene, polypropylene, polycarbonate and the like), bottles, vials, paper, envelopes and the like. Additionally, the kits of the invention can contain instructional material for the simultaneous, sequential or separate use of the different components contained in the kit. The instructional material can be in the form of printed material or in the form of an electronic support capable of storing instructions such that they can be read by a subject, such as electronic storage media (magnetic disks, tapes and the like), optical media (CD-ROM, DVD) and the like. Alternatively or in addition, the media can contain internet addresses that provide the instructional material.

In order that the invention may be readily understood and put into practical effect, particular preferred embodiments will now be described by way of the following non-limiting examples.

### EXAMPLES

### Example1

### Development of Bicyclic Inhibitors Targeting PD-L1 Nuclear Post-Translational Modifications

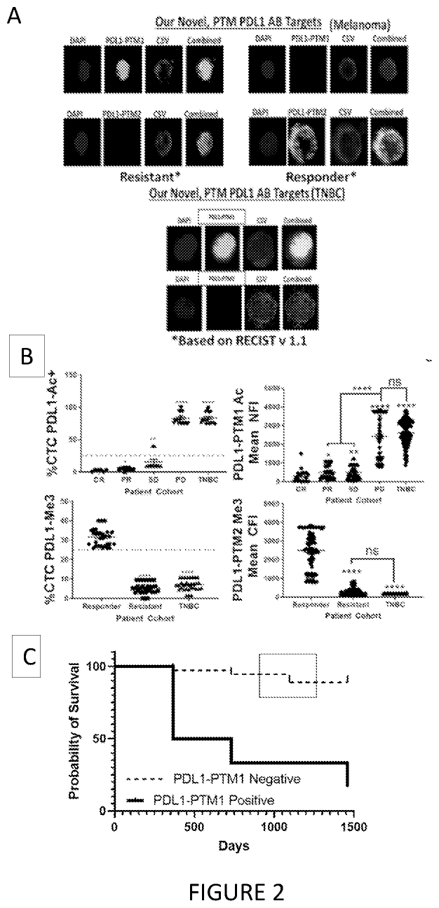

The present inventor carried out an alanine walk to identify critical residues within the acetylation/de-methylation and nuclear localization sequence of PD-L1. A series of residues were identified that when mutated to alanine reduce effective inhibition of the number of metastatic initiating cells (MICs) by more than 50% (see, Table 1). The present inventor also identified two residues that when mutated to an alanine increased effective inhibition of MIC number as compared to control. Based on optimized sequences three cyclic peptide inhibitors were generated (DL-1, DL-2, and DL-3). DL-1 and 2 were taken forward as both these constructs preserved the NLS motif and the critical K263 residue which is subject to post-translational modification (PTM), whereas DL-3 disrupted the core NLS motif and therefore was not considered viable. The DL-2 construct contains linker residues Ala-Cys at the front of the NLS motif and this allows the core critical residues to be accessible in the bicyclic peptide's 3D dimensional structure (see, FIG. 2B). DL-1 has Cys-Gly linker residues at the end of the sequence, which radically changes the 3D structure of the bicyclic peptide leaving the critical residues in the front end and the NLS less accessible. The present inventors therefore hypothesised that DL-2 should work whereas DL-1 may not be as effective as DL-2 due to the linker region at the C-terminal end of sequence disrupting the overall 3D structure and accessibility to the critical motifs/residues.

TABLE 5

| Alanine Walk | % Inhibition |
|---|---|
| TFIFRLRKGRMADVKK | 100 |
| TFIFRLRKGRMMAVKK | 100 |
| TFIFRLRKGRMMDAKK | 81.9 |
| AFIFRLRKGRMMDVKK | 80.13 |
| TAIFRLRKGRMMDVKK | 74.35 |
| TFIFRLRKGRAMDVKK | 74.35 |
| TFIFALRKGRMMDVKK | 71.67 |
| TFIFRLRKGAMMDVKK | 62.69 |
| TFIFRLRKGRMMDVKA | 58.03 |
| TFIFRLRKGRMMDVAK | 48.7 |
| TFIFRLAKGRMMDVKK | 43.98 |
| TFIFRLRKARMMDVKK | 41.38 |
| TFIFRARKGRMMDVKK | 35.6 |
| TFAFRLRKGRMMDVKK | 30.2 |
| TFIARLRKGRMMDVKK | 4.4 |
| TFIFRLRAGRMMDVKK | 0 |

### Materials & Methods

### Flow Cytometry for Alanine Walk

FACS (Fluorescence-activated cell sorting) was performed on single-cell suspensions treated with a suite of alanine walk peptide inhibitors. Cells were stained an array of antibodies to target the metastatic initiating cell (MIC) signature including CD44, CD24, SNAIL, ABCB5, CSV and Hoechst dye to monitor cell viability. The FACS gating strategy used is adopted and modified from our signature for metastatic initiating cells (MICs), which sorted cells based on the MIC, therapeutic resistant phenotype, shown to be associated with metastatic and aggressive cancer cells. A flow cytometry gating strategy was always included in the analysis to exclude dead cells (Hoechst-negative cells). Flow cytometry acquisition was performed on single cell suspensions using BLSR II. Analysis was performed using FlowJo software.

### Example 2

### PD-L1 NLS PTM Motifs are Indicative of Cancer Treatment Responsiveness

The present inventors next employed their novel biomarker discovery, a novel PD-L1 acetylation/methylation biomarker that stratifies immunotherapy resistant and responsive metastatic cancer patients. Many cell signalling proteins have recently been described as having nuclear gene regulatory roles. Many proteins implicated in cancer development and progression are exquisitely regulated by PTMs that dictate distinct transcriptional outcomes. Previous data demonstrates that PTMs also control the activity of immune checkpoint proteins to mediate resistance to immuno- and chemotherapy. To identify potential immune checkpoint regulators, the present inventors performed in silico screening of multiple immune checkpoint proteins harbouring:

(i) putative nuclear localisation sequences (NLS); and
(ii) epigenetic-based PTM signatures.

Based on PD-L1 motif analysis, the present inventors identified specific acetylation/methylation motifs at lysine 263 within a high score NLS. Cell staining studies with antibodies targeting these motifs determined the presence of PDL1-PTM1 (acetylation), which is enriched in immune-resistant cancer cell nuclei, and PDL1-PTM2 (methylation) which is enriched in immune-responsive cancer cells in the cytoplasm/at the cell surface (FIG. 2A). These novel nPD-L1-PTM variants were exclusively enriched in intrinsically resistant aggressive “cold tumours” (such as TNBCs and prostate cancers) as well as resistant “hot tumours” (such as melanomas and lung cancers) and were associated with immunotherapy responses (FIG. 2B). Recent studies also support a nuclear role for PD-L1 in controlling tumour growth and proliferation.

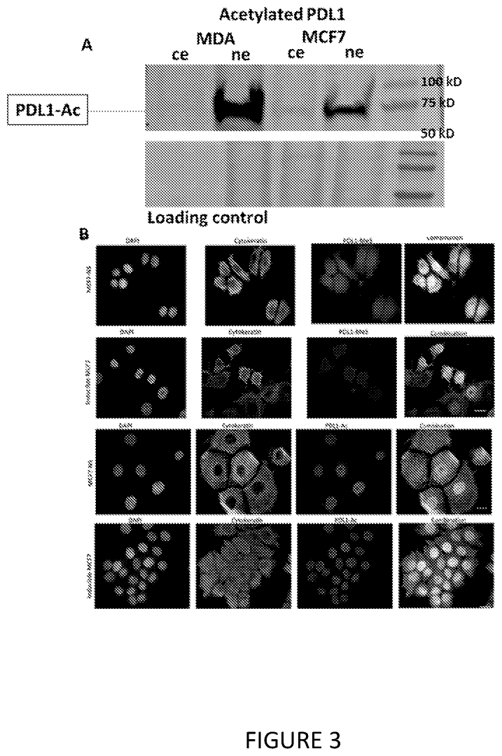

Based on these data, the present inventor developed a liquid biopsy nPD-L1 diagnostic biomarker to identify immunotherapy patients likely to respond to immunotherapy (FIG. 2). Five novel affinity-purified antibodies that target PTM1 (263-lysine-acetylation) and PTM2 (263-lysine-tri-methylation) in PD-L1 have been raised. Notably, this region is not targeted by commercially available PD-L1 diagnostic antibodies (e.g., 73-10, 28-8, SP142, CAL-10). The specificity for the target sequence was confirmed by extensive ELISA assays, which showed that the custom antibodies targeting PTM1 (263-lysine-acetylation) or PTM2 (263-lysine-tri-methylation) do not detect the unmodified motif. Additionally, in vitro peptide blocking assays with peptide mimics for specific PTMs blocked our corresponding novel PD-L1-targeting antibodies, whereas the peptide mimics for specific PTMs did not affect commercial antibody binding. We have demonstrated the specificity of our antibody in our inducible mesenchymal model (MCF7) in high throughput screening (FIG. 3A) and immunoblot analysis (FIG. 3B). These data demonstrate that the acetylated form of PD-L1 is indeed the nuclear biased variant.

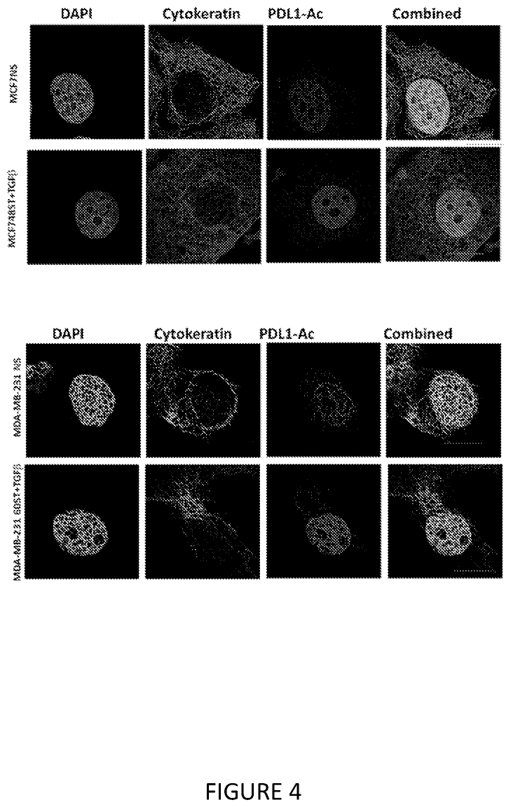

To further confirm the nuclear localisation of our PD-L1-PTM1 specific antibody the present inventors carried out super resolution imaging using the ANDOR spinning disc super resolution microscope. This imaging data (FIG. 4) clearly depicts the nuclear localisation of PD-L1-PTM1 in both the TNBC cell line MDA-MB-231, and in an inducible mesenchymal MCF7 model.

### Example 3

### PD-L1-Me3 and PD-L1-Ac Expression in TNBC Brain Metastatic Lesions

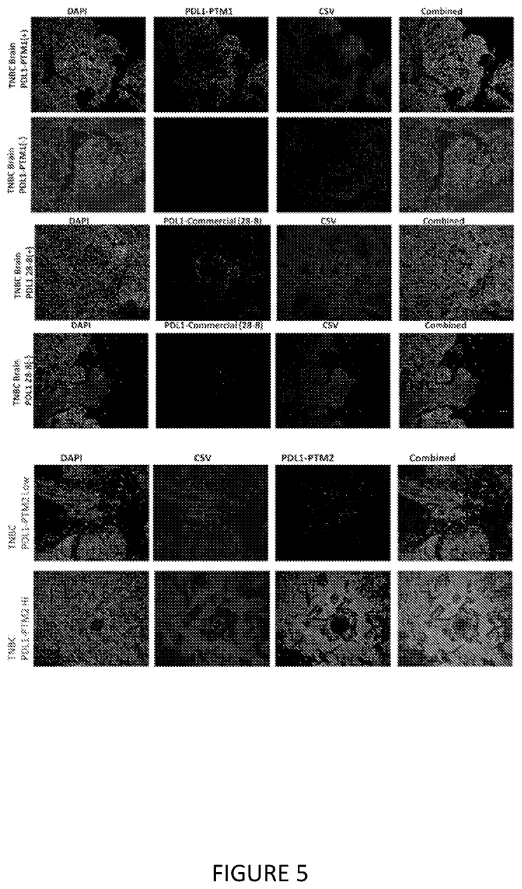

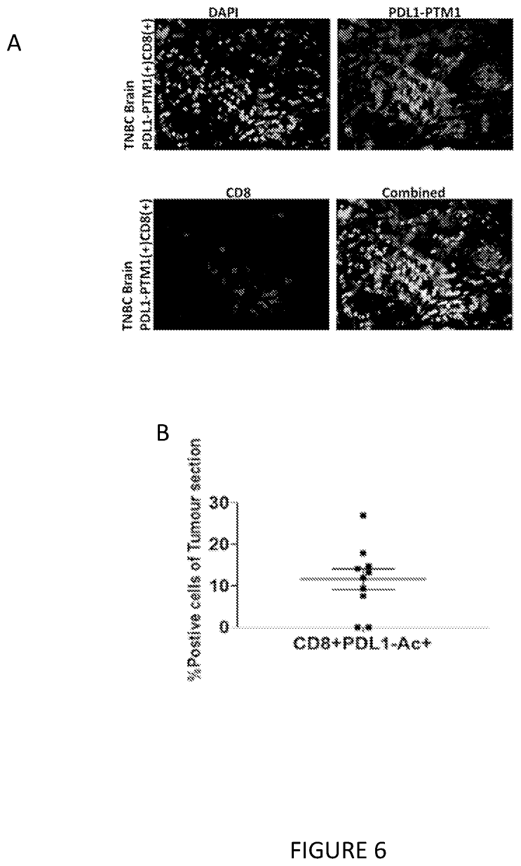

The present inventors also confirmed the presence of both nuclear PD-L1-PTM1 (PD-L1-Ac) and cytoplasmic PD-L1-PTM2 (PD-L1-Me3) in brain metastatic lesions in metastatic TNBC patients. These data indicate the potential therapeutic target of PD-L1-PTM1 or PTM2 in treating TNBC patients with brain metastatic disease, a cohort of patients difficult to treat and resistant to immunotherapy (FIG. 5). Additionally, the present inventors have also observed the infiltrating CD8+ T cells within tumour metastatic lesions in the brain of TNBC patients are positive for PD-L1-PTM1 (PD-L1-Ac) expression (see, FIG. 6). These results indicate that these data may also be indicative of dysfunctional CD8+ T cells.

### Summary

PD-L1 exists in a variety of post-translationally modified forms. We have identified two key PTMs: tri-methylation and acetylation at lysine 263.

Acetylation (Ac) occurs on the predominantly nuclear PD-L1 isoform, with little cytoplasmic expression and no cell surface expression. Characterisation in patient cohorts: Predominantly associated with patients resistant to immunotherapy and progressive disease.

Methylation (Me3) occurs predominantly on the cytoplasmic and cell surface isoforms, with little nuclear expression. Characterisation in patient cohorts: Predominantly associated with patients responsive to immunotherapy. Therefore, PD-L1 tri-methylation, offers an avenue for new opportunities for targeting PD-L1 to the cell surface for effective immunotherapy.

The present inventors have developed bicyclic inhibitors capable of competitively targeting this nuclear axis.

Moreover, the present inventors determined that the PD-L1 nuclear localization motif is conserved across multiple species, and unique to PD-L1:

>sp|Q9NZQ7|PD1L1_HUMAN Programmed cell death 1 ligand 1 OS = Homo sapiens
OX = 9606 GN = CD274 PE = 1 SV = 1
Human:
LTFIFRLRKG-RMMDVKKCGIQDTRSKK
Mouse:
LLF---LRKQVRMLDVEKCGVEDTSSKN (Identities: 50%, Positives: 79%)
Pig:
TAIFCLKRNV-RMMDVEKCGSRDMKSEK (Identities: 58%, Positives: 78%)
Marmot:
LTILFCLRKNVRMLDVENGGIQDINSRK (Identities: 62%, Positives: 75%)
Cat:
LKKRDGISFIAVVPTGHMGKRMGGCCCH (Identities: 6%, Positives: 0%)
Dog:
LAVTFCLKKHGRMMDVEKCCTRDRNSKK (Identities: 61%, Positives: 75%)
Gorilla:
LTFIFCLRKG-RMNDVKKCGIQDTNSKK (Identities: 85%, Positives: 90%)
Phesus:
LTFIFYLRKG-RMMDMKKSGIRVTNSKK (Identities: 85%, Positives: 90%)
Chimp:
LTFIFCLRKG-RMMDVKKCGIQDTNSKK (Identities: 97%, Positives: 97%)

### Example 4

### Efficacy of Bicyclic Peptide Inhibitors

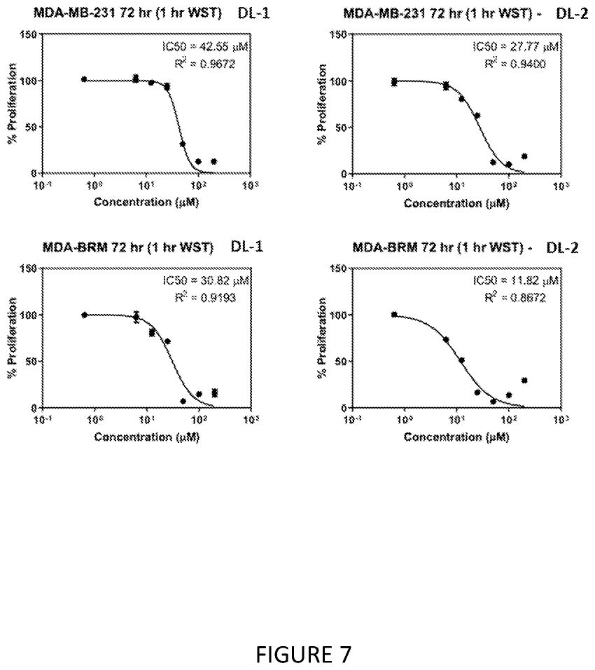

The present inventors next took advantage of their custom novel antibody tools to probe the effectiveness of candidate bicyclic inhibitors targeting the nuclear form of PD-L1-PTM1. First, the inventors examined the impact of DL-1 and DL-2 on the proliferation of the triple negative breast cancer (TNBC) cell line MDA-MB-231, or its brain cancer clone MDA-MB-231-Br. Strikingly, although both bicyclic peptide inhibitors (DL-1 and DL-2) were able to inhibit proliferation of the TNBC cancer cell lines, DL-2 was able to inhibit at 2-3-fold higher potency than DL-1 (FIG. 7).

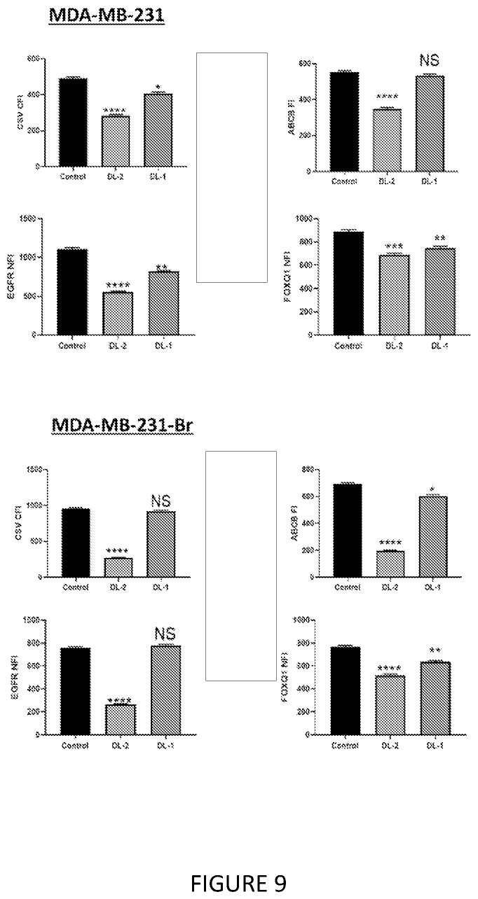

Next, mesenchymal regulators of metastatic burden and a resistant, stem-like signature were examined via high resolution digital pathology, after treatment with the bicyclic inhibitor DL-1 or DL-2 (FIG. 9).

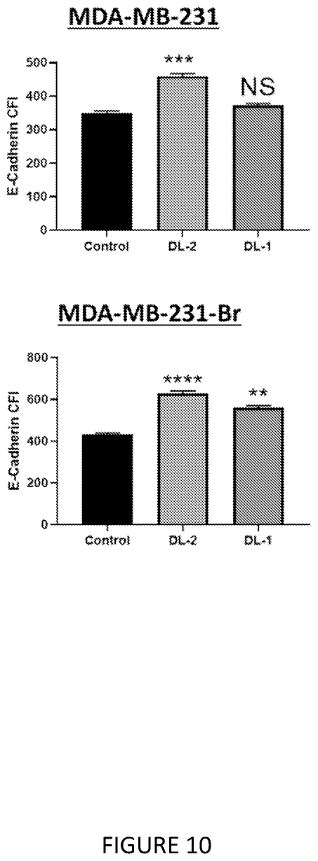

Analysis revealed that DL-2 was able to significantly inhibit all mesenchymal stem-like resistant protein markers in both MDA-MB-231 and MDA-MB-231-Br TNBC cell lines. These data demonstrate clear efficacy for targeting nuclear PD-L1-Ac (PTM1) with the lead bicyclic peptide candidate, DL-2. Interestingly, the present inventors found that treatment with DL-2 and (to a lesser extend DL-1) was also able to induce expression of the epithelial marker E-Cadherin in both MDA-MB-231 and MDA-MB-231-Br TNBC cell lines (FIG. 10).

Next, the present inventor examined the ability of DL-2 to induce cell surface or cytoplasmic expression of PD-L1-Me3. It was hypothesized that inducing cell surface/cytoplasmic expression of PD-L1-Me3 would also be linked with an immunotherapy responsiveness signature, by the increased expression of cell surface PD-L1-Me3 providing a target for anti-PD-L1 immunotherapies to hit. This would also be in agreement with the data presented above.

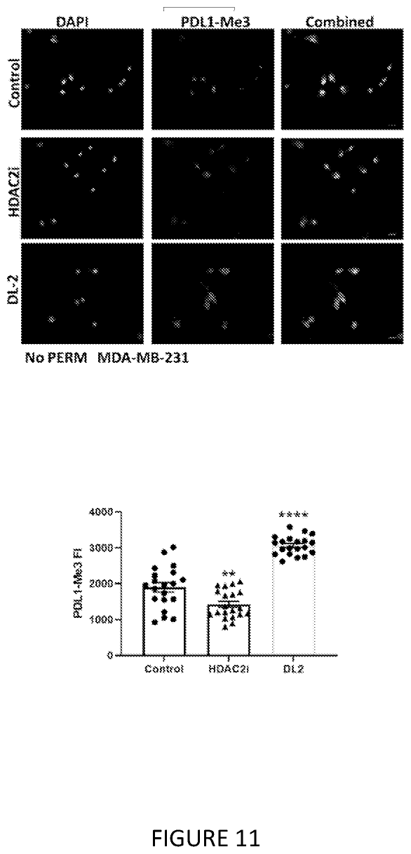

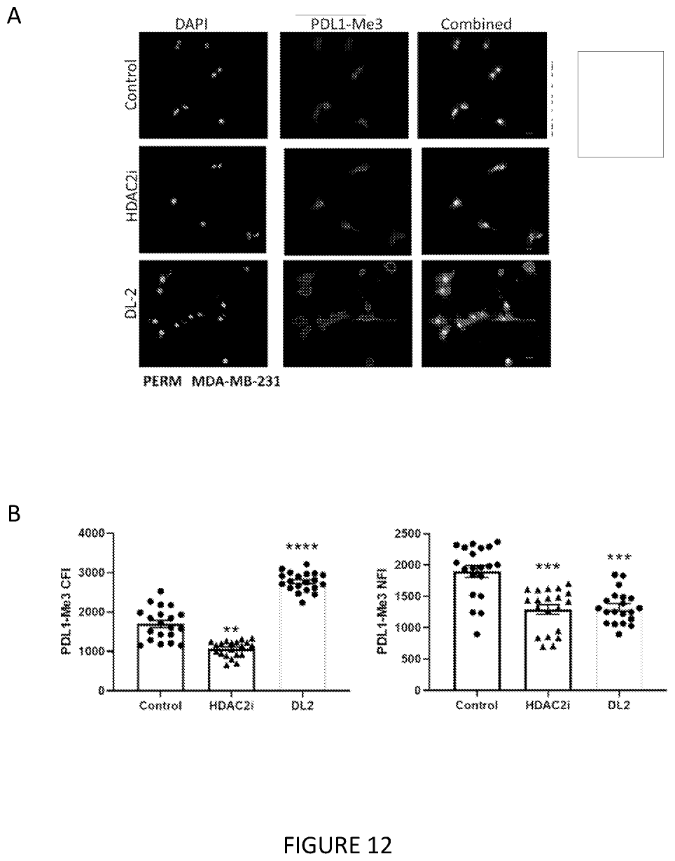

The present inventors first examined the effect of DL-2 inhibition with a HDAC2i in non-permeabilized cells, which would prevent the de-acetylation of PD-L1 and theoretically increase the acetylation of PD-L1 (and reduce the methylation of PD-L1) at the lysine 263 PTM-NLS motif. Using high resolution digital pathology, we found that treatment with DL-2 induced an significant upregulation in the expression of cell surface PD-L1-Me3 (FIG. 11). An almost identical pattern was seen in permeabilized cells, with DL-2 abrogating the expression of nuclear PD-L1-Me3 and significantly upregulating cytoplasmic/cell surface P-DL1-Me3 in the permeabilized MDA-MB-231 cells (FIG. 12).

### Summary

The present inventors have identified a novel bicyclic inhibitor, DL-2, able to target and inhibit proliferation of metastatic cancers as well inhibiting expression of mesenchymal markers including CSV (Cell surface Vimentin), EGFR, FOXQ1 and ABCBS expression. DL-2 was also able to induce expression of the epithelial marker E-cadherin. This data sets indicate that DL-2 inhibition is able to re-program a mesenchymal, resistant signature cancer cell to a epithelial responsive cancer cell by targeting the nuclear axis of PD-L1 with our novel, bicyclic inhibitors. This also indicates that as DL-2 will induce cell surface expression of PD-L1 (P-DL1-PTM2/PD-L1-Me3) that treated cells will be amenable to sequential of combination immunotherapy, with the increase cell surface PD-L1 providing a target for anti-PD-L1 immunotherapy to act on. This in combination with our TNBC brain metastatic data indicate that targeting the nuclear PDL1 axis may abrogate resistance to immunotherapy and apply to patients with heavy metastatic burden such as in the case of brain metastatic diseases.

### Example 5

Previous studies have revealed that patients resistant to immunotherapy have a form of PD-L1 that is internalised to the nucleus. To provide an alternative treatment for these patients, the present inventors have successfully and specifically targeted nuclear PD-L1 with a novel bicyclic peptide mimetic, DL-2, which prevents PD-L1 from entering the nucleus.

RNA-sequencing (RNA seq) data were obtained from human breast cancer cell line MCF-7 with and without DL-2 treatment. A total of 9 samples from 4 experimental groups were collected, as follows:
Control (non-stimulated control, Ctrl) (n=3);
Stimulated (PMA stimulated control) (n=3);
DL-2 (treated with DL-2 and stimulated with PMA) (n=3);

For the DL-2 treatment group, the MCF-7 cells were exposed to DL-2 first, then stimulated with PMA thereafter.

The aim of this script is to perform differential expression analysis and pathway analysis between:
Stimulated vs. Control to determine which genes were impacted by stimulation by PMA.
DL-2 vs. Stimulated: to determine which genes were impacted by the DL-2 PD-L2 bicyclic peptide mimetic.

The present inventors determined genes that were differentially expressed between the DL-2 group and the stimulated control group.

### Differential Expression Analysis between Stimulated and Control Groups

Differential expression analysis of Stimulated and Control groups resulted in the following numbers of differentially expressed genes (DEGs):
2219 genes with an FDR<0.01 and absolute logFC>1;
2222 genes with an FDR<0.05 and absolute logFC>1;
4072 genes with an FDR<0.05 and absolute logFC>0.584963 (FC 1.5);
6385 genes with an FDR<0.05 and absolute logFC>0.321928 (FC 1.25);
8096 genes with an FDR<0.01 (no logFC filter);
8779 genes with an FDR<0.05 (no logFC filter).

By limiting the results to the DEGs with an FDR<0.05, the present inventors further elucidated
2191 genes with logFC>0.584963 (up-regulated in Stimulated samples, FC 1.5);
1881 genes with logFC<−0.584963 (down-regulated in Stimulated samples, FC −1.5).

Differential expression analysis between DL-2 and Stimulated groups

Differential expression analysis of the DL-2 and Stimulated groups resulted in the following numbers of differentially expressed genes (DEGs):
48 genes with an FDR<0.01 and absolute logFC>1;
49 genes with an FDR<0.05 and absolute logFC>1;
779 genes with an FDR<0.05 and absolute logFC>0.584963 (FC 1.5);
3439 genes with an FDR<0.05 and absolute logFC>0.321928 (FC 1.25);
6094 genes with an FDR<0.01 (no logFC filter);
7305 genes with an FDR<0.05 (no logFC filter).

Furthermore, by limiting the results to the DEGs with an FDR<0.05, the present inventors discovered:
385 genes with logFC>0.584963 (up-regulated in DL-2 samples, FC 1.5);
394 genes with logFC<−0.584963 (down-regulated in DL-2 samples, FC −1.5).

These results are presented in Tables 6 and 7, below.

### Materials and Methods

### IFA Microscopy

To examine the signature of CD8, CSV, EGFR, ABCB5, FOXQ1, commercial PD-L1-28-8, nuclear PD-L1, cytoplasmic PDL1 in MDA-MB-231 TNBC cells/TNBC brain cancer clone cells (untreated or treated with bicyclic inhibitors targeting nuclear PD-L1-PTM1). MDA-MB-231/MDA-MB-231-Br cells were permeabilised by incubating with 0.5% Triton X-100 for 15 min, blocked with 1% BSA in PBS and were probed with either CSV (Mouse host), CD8 (mouse host), EGFR (Rabbit host), ABCB5 (Goat Host), ALDH1A (Rabbit Host), NODAL (Goat Host), nuclear PDL1 (rabbit host), cytoplasmic PD-L1 (rabbit host) visualized with a donkey anti-rabbit AF 488, anti-mouse AF 568, donkey anti-goat 647. Cover slips were mounted on glass microscope slides with ProLong Glass Antifade reagent (Life Technologies). Protein targets were localised by digital pathology laser scanning microscopy. Single 0.5 μm sections were obtained using a ASI Digital pathology microscope using 100× oil immersion lens running ASI software. The final image was obtained by averaging four sequential images of the same section. Digital images were analyzed using automated ASI software (Applied Spectral Imaging, Carlsbad, CA) to determine the distribution and intensities automatically with automatic thresholding and background correction of the average nuclear fluorescence intensity (NFI), allowing for the specific targeting of expression of proteins of interest. Digital images were also analysed using ImageJ software (ImageJ, NIH, Bethesda, MD, USA) to determine the total cell fluorescence or cell surface only fluorescence for non-permeabilised cells. Digital images were analysed using ImageJ software (ImageJ, NIH, Bethesda, MD, USA) to determine either the Total Nuclear Fluorescent Intensity (TNFI), the Total Cytoplasmic Fluorescent Intensity (TCFI). ImageJ software with automatic thresholding and manual selection of regions of interest (ROIs) specific for cell nuclei was used to calculate the Pearson's co-efficient correlation (PCC) for each pair of antibodies. PCC values range from: −1=inverse of co-localisation, 0=no co-localisation, +1=perfect co-localisation. The Mann-Whitney nonparametric test (GraphPad Prism, GraphPad Software, San Diego, CA) was used to determine significant differences between datasets.

### Andor Spinning DISC Super Resolution Microscopy

Samples were prepared as per IFA microscopy for imaging under oil immersion for super resolution with the ANDOR spinning disc microscope.

### Opal Tissue Microscopy

To examine the signature of nuclear PD-L1-PTM1, PTM2, PD-L1-28-8, CSV (cancer cell marker) the OPAL staining kit for automatic staining use the automatic bondrx platform was used following them manufactures directions. Proteins were then mounted and localised as described in IFA microscopy.

### Cell Culture methods

All breast cancer cell lines used were sourced from ATCC. MDA-MB-231 or MDB-MB-231-Br cell lines were maintained and cultured in DMEM (Invitrogen) supplemented with 10% FBS, 2 mM L-glutamine, and 1% PSN. MCF-7 cells were stimulated with 1.29 ng/mL phorbol 12-myristate 13-acetate (PMA) (Sigma-Aldrich) or 5 ng/ml recombinant TGF-β1 (R&D Systems) for 60 hours. For inhibitor studies, 2×10⁵ cells were seeded in 2 mL complete media in 6 well plates and incubated overnight at 37° C./5% CO₂. Cells were treated with PD-L1 bicyclic peptides targeting the nuclear axis PD-L1, HDAC2i or vehicle control. For qPCR analysis, at each time point, adherent cells were trypsinized, washed and cell pellets stored at −80° C. until further processing. For microscopy studies, 4×10⁴ cells were seeded on coverslips and treated with inhibitors as described above. At each timepoint, coverslips were washed, fixed with 3.7% formaldehyde (Sigma) and stored at 4° C. until processing. For qPCR, cells were lysed in 1 mL Tri-reagent (Sigma) while for microscopy studies, cells were fixed in 3.7% formaldehyde and collected by trypsinization and cell scraping.

TABLE 6
TOP UP-REGULATED DEGS (FDR < 0.05, LOGFC > 0.584963)

| | Associated | | | | | |
|---|---|---|---|---|---|---|
| Gene Name | Gene Name | logFC | logCPM | F | PValue | FDR |
| ENSG00000175899 | A2M | 1.4605786 | 4.362961 | 465.7864 | 1.730187e−12 | 6.190032e−09 |
| ENSG00000134827 | TCN1 | 2.0286269 | 2.543160 | 476.4721 | 1.472920e−12 | 6.190032e−09 |
| ENSG00000117984 | CTSD | 1.0785289 | 11.479127 | 425.8496 | 3.265460e−12 | 8.762045e−09 |
| ENSG00000159167 | STC1 | 0.7264964 | 7.130277 | 344.1711 | 1.465676e−11 | 3.146220e−08 |
| ENSG00000123131 | PRDX4 | 0.6510737 | 6.342907 | 328.9059 | 2.015181e−11 | 3.303642e−08 |
| ENSG00000185499 | MUC1 | 0.9921248 | 5.016511 | 325.7820 | 2.154616e−11 | 3.303642e−08 |
| ENSG00000166557 | TMED3 | 0.6673394 | 8.018117 | 304.5815 | 3.450792e−11 | 3.819628e−08 |
| ENSG00000196136 | SERPINA3 | 1.0084230 | 7.915897 | 305.7660 | 3.358427e−11 | 3.819628e−08 |
| ENSG00000107562 | CXCL12 | 1.1551081 | 4.224435 | 283.1920 | 5.735814e−11 | 4.735576e−08 |
| ENSG00000145050 | MANF | 0.8171654 | 6.372911 | 273.6680 | 7.278154e−11 | 5.579745e−08 |
| ENSG00000123989 | CHPF | 0.6073052 | 8.617671 | 268.7466 | 8.256905e−11 | 5.762237e−08 |
| ENSG00000168000 | BSCL2 | 0.9848617 | 6.030146 | 260.5884 | 1.022801e−10 | 6.457485e−08 |
| ENSG00000204386 | NEU1 | 0.6165229 | 7.892050 | 254.3756 | 1.209117e−10 | 6.489821e−08 |
| ENSG00000149541 | B3GAT3 | 0.8185780 | 7.051983 | 257.4847 | 1.111450e−10 | 6.489821e−08 |
| ENSG00000138185 | ENTPD1 | 0.8955543 | 4.203179 | 254.3694 | 1.209321e−10 | 6.489821e−08 |
| ENSG00000117410 | ATP6V0B | 0.6470462 | 7.666058 | 248.0773 | 1.438423e−10 | 7.351713e−08 |
| ENSG00000074842 | C19orf10 | 0.7917153 | 6.414474 | 245.5424 | 1.544381e−10 | 7.534474e−08 |
| ENSG00000141753 | IGFBP4 | 0.6163633 | 7.997707 | 242.2437 | 1.695808e−10 | 7.838453e−08 |
| ENSG00000002586 | CD99 | 0.6829216 | 6.816162 | 239.7823 | 1.819833e−10 | 7.838453e−08 |
| ENSG00000115457 | IGFBP2 | 0.7385329 | 7.984140 | 239.6691 | 1.825783e−10 | 7.838453e−08 |
| ENSG00000231925 | TAPBP | 0.5967889 | 8.163352 | 231.5709 | 2.314703e−10 | 8.281234e−08 |
| ENSG00000166794 | PPIB | 0.8357538 | 9.116189 | 235.5659 | 2.057038e−10 | 8.281234e−08 |
| ENSG00000160182 | TFF1 | 1.2527473 | 10.951120 | 232.2850 | 2.266070e−10 | 8.281234e−08 |
| ENSG00000116299 | KIAA1324 | 0.6323565 | 6.348370 | 225.9583 | 2.741132e−10 | 8.915324e−08 |
| ENSG00000102683 | SGCG | 1.1259670 | 4.701178 | 226.1477 | 2.725363e−10 | 8.915324e−08 |

TABLE 7
TOP DOWN-REGULATED DEGS (FDR < 005, LOGFC <− 0.584963)

| | Associated | | | | | |
|---|---|---|---|---|---|---|
| Gene Name | Gene Name | logFC | logCPM | F | P Value | FDR |
| ENSG00000100985 | MMP9 | −1.4304297 | 5.570659 | 695.3896 | 9.931439e−14 | 1.065941e−09 |
| ENSG00000259741 | ZMYND11 | −2.8826537 | 3.976913 | 231.7783 | 2.300458e−10 | 8.281234e−08 |
| ENSG00000198431 | TXNRD1 | −0.7667286 | 8.968334 | 171.2586 | 1.819054e−09 | 2.380965e−07 |
| ENSG00000147883 | CDKN2B | −0.5981580 | 7.146393 | 167.9811 | 2.072848e−09 | 2.586963e−07 |
| ENSG00000120690 | ELF1 | −0.7207348 | 7.329006 | 166.9642 | 2.159617e−09 | 2.633996e−07 |
| ENSG00000184254 | ALDH1A3 | −0.9247774 | 4.505860 | 150.7770 | 4.287426e−09 | 4.036574e−07 |
| ENSG00000039560 | RAI14 | −0.8090645 | 5.072774 | 147.9015 | 4.877051e−09 | 4.283972e−07 |
| ENSG00000137710 | RDX | −0.9705274 | 6.290825 | 147.5099 | 4.964276e−09 | 4.283972e−07 |
| ENSG00000119326 | CTNNAL1 | −0.5942752 | 5.411627 | 139.5355 | 7.191551e−09 | 5.259202e−07 |
| ENSG00000119922 | IFIT2 | −1.2131260 | 3.038698 | 130.7728 | 1.105784e−08 | 6.705299e−07 |
| ENSG00000131374 | TBC1D5 | −0.8969911 | 5.796234 | 126.8949 | 1.348988e−08 | 7.620363e−07 |
| ENSG00000187134 | AKR1C1 | −1.3242964 | 1.803872 | 124.6363 | 1.518442e−08 | 8.393826e−07 |
| ENSG00000109452 | INPP4B | −0.6381166 | 6.893658 | 124.0682 | 1.564794e−08 | 8.568843e−07 |
| ENSG00000168386 | FILIP1L | −0.7562886 | 4.493788 | 123.2192 | 1.637099e−08 | 8.743791e−07 |
| ENSG00000118200 | CAMSAP2 | −0.7467627 | 6.368909 | 122.2687 | 1.722588e−08 | 8.931661e−07 |
| ENSG00000111859 | NEDD9 | −0.7516218 | 5.352839 | 119.7960 | 1.969804e−08 | 9.698121e−07 |
| ENSG00000147526 | TACC1 | −0.6077168 | 6.567683 | 117.2682 | 2.265108e−08 | 1.052442e−06 |
| ENSG00000053254 | FOXN3 | −0.7499869 | 4.955113 | 115.4534 | 2.508263e−08 | 1.131142e−06 |
| ENSG00000114978 | MOB1A | −0.6723104 | 6.351804 | 114.1979 | 2.693850e−08 | 1.184963e−06 |
| ENSG00000185947 | ZNF267 | −0.6577183 | 4.281608 | 113.3444 | 2.828947e−08 | 1.195397e−06 |
| ENSG00000109790 | KLHL5 | −1.0275478 | 6.306190 | 111.0700 | 3.228156e−08 | 1.296982e−06 |
| ENSG00000117036 | ETV3 | −0.6423534 | 5.002629 | 110.3715 | 3.363344e−08 | 1.332058e−06 |
| ENSG00000096717 | SIRT1 | −0.7444282 | 4.713947 | 109.7598 | 3.487056e−08 | 1.351140e−06 |
| ENSG00000151012 | SLC7A11 | −0.8545189 | 5.268820 | 108.8133 | 3.688778e−08 | 1.413988e−06 |
| ENSG00000112531 | QKI | −0.8595209 | 6.058521 | 107.3872 | 4.018327e−08 | 1.471969e−06 |

### WST-1 Methods

MDA-MB-231 cells were seeded at 4×10³ cells/well into a 96-well flat bottom tissue culture plate in a final volume of 100 ul and left to adhere for 24 hours at 37° C. and 5% CO2. Cells were then treated with inhibitors for 72 hours at a final concentration of 200, 100, 50, 25, 12.5 or 6.25 μM. Following inhibition, media was removed and replaced with 100 μL/well of WST-1 cell proliferation reagent (Sigma-Aldrich, Cat# 11644807001) at 1:10 final dilution. Absorbance was recorded at 450 nm at 1, 2, 3, and 4 hour incubation periods using a microplate spectrophotometer (with 30 sec mix time). Data represent a single independent experiment performed in triplicate, results are graphed as mean +/− standard error (SE).

### RNA-Seq Library Preparation: TruSEQ Stranded mRNA (polyA Capture)

RNA-seq data were generated, fastq data were downloaded to the QIMR Berghofer server, and then archived to the HSM by Scott Wood. Sequence reads were trimmed for adapter sequences using Cutadapt (version 1.9; Martin (2011)) and aligned using STAR (version 2.5.2a; Dobin et al. (2013)) to the GRCh37 assembly with the gene, transcript, and exon features of Ensembl (release 70) gene model. Quality control metrics were computed using RNA-SeQC (version 1.1.8; DeLuca et al. (2012)) and expression was estimated using RSEM (version 1.2.30; Li and Dewey (2011)).

### Quality Control

The quality control of RNA-seq samples is an important step to guarantee quality and reproducible analytical results. RNA-SeQC was run for this purpose.

### Data Normalisation

The aim of normalisation is to remove differences between samples based on systematic technical effects to warrant that these technical biases have a minimal effect on the results. The library size is important to correct for as differences in the initial RNA quantity sequenced will have an impact on the number of reads sequenced. Differences in RNA sequence composition occurs when RNAs are over-represented in one sample compared to others. In these samples, other RNAs will be under-sampled which will lead to higher false-positive rates when predicting differentially expressed genes.

### Normalisation Methods

In our analysis, we corrected for library size by dividing each sample's gene count by million reads mapped. This procedure is a common approach known as counts per million (CPM). We further corrected for differences in RNA composition using a method proposed by Robinson and Oshlack (2010a) called trimmed mean of M values (TMM). We used the function calcNormFactors( ) from the edgeR package (Robinson, McCarthy, and Smyth (2010b)) to obtain TMM factors and used these to correct for differences in RNA composition.

### Principal Component Analysis

Principal Component Analysis (PCA) is a dimension reduction method that decomposes the original, often correlated, variables (here read counts) into a set of independent (uncorrelated) variables called Principal Components (PCs). The first PC accounts for as much of the variability as possible, the second PC is independent of the first PC and accounts for second most variability in the data. The same applies for the remaining PCs. We used the PCA implementation from the function prcomp( ). PCA analysis is performed to identify the main cause of variance in the data. Ideally, we would like to the see the samples clustered by the experimental grouping. The extraction of protein-coding genes leaves 17329 for subsequent analysis. After keeping only genes with >5 CPM in >2 samples, we have 10733 genes left for analysis.

### Differential Expression Analysis

Differential expression (DE) analysis was performed using the R package edgeR (Robinson, McCarthy, and Smyth (2010b)). Note that the inputs for DE analysis are the filtered but not normalised read counts, since edgeR performs normalisation (library size and RNA composition) internally. The glmQLFit( ) function was used to fit a quasi-likelihood negative binomial generalised log-linear model to the read counts for each gene. Using the glmQLFTest( ) function, we conducted gene-wise empirical Bayes quasi-likelihood F-tests for a given contrast. As per the edgeR user's guide, “the quasi-likelihood method is highly recommended for differential expression analyses of bulk RNA-seq data [versus the likelihood ratio test] as it gives stricter error rate control by accounting for the uncertainty in dispersion estimation”.

### Example 5

### PDL1 and Importin Mechanism of Action

The data presented above clearly demonstrate that the PDL1-PTM1 is predominately located in the nucleus of immunotherapy resistant cancers, whereas PDL1-PTM2 is localised in the cytoplasm of cancers responsive to immunotherapy. To further confirm the mechanism of action of PDL1-PTM1 (i.e., acetylation) and PDL1-PTM2 (i.e., methylation) the inventors carried out structural modelling to establish the potential of these PTMs on the interaction with the PD1 and importin nuclear machinery. 3A depicts the mode of action of PDL1 PTM forms in either cancer progression or response to immunotherapy. This structural analysis shows that methylation of PDL1 (PDL1-PTM2) prevents interaction between PD1 and PDL1 acting similar to an immunotherapy. Structural analysis also revealed that methylation of this NLS motif at position lysine 263 inhibits the interaction with the importin pathway whereas PDL1-PTM1 is enriched in the nucleus of progressive or immunotherapy resistant cancer.

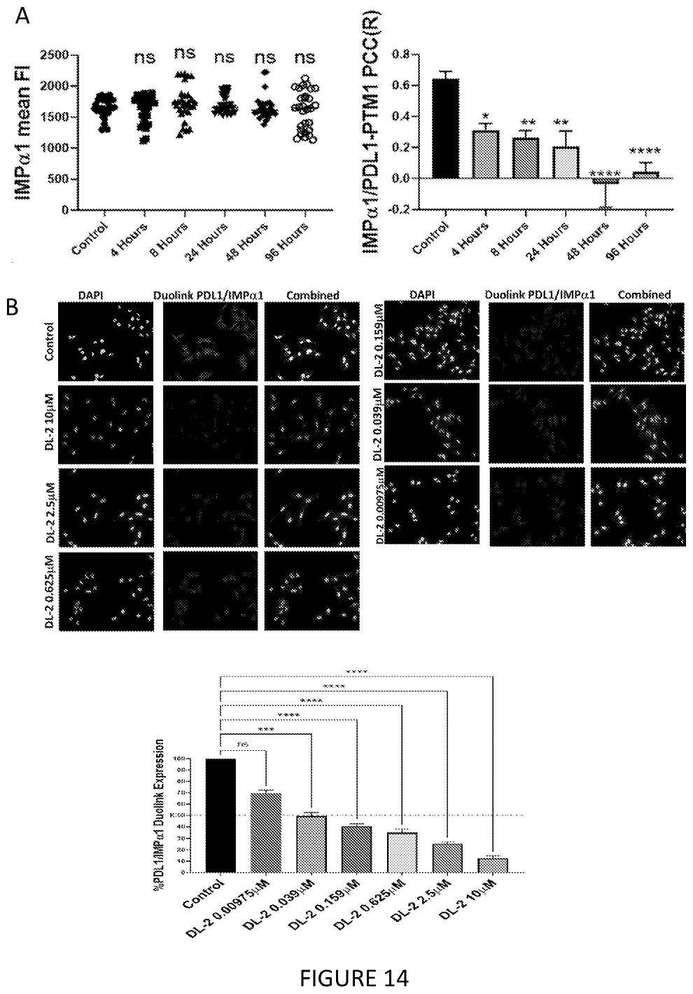

The present inventor designed DL-2 to inhibit the nuclear translocation of PDL1, via interference with the importin pathway. The ability of DL-2 to interfere with the interaction of PDL1-PTM1 and importin-α (IMPα) in a TNBC human cancer cell line by monitoring the co-localization (PCC) of PDL1-PTM1 and IMPα1. DL-2 was able to significantly impair the PCC or degree of co-localization of PDL1-PTM1 and IMPα1 up to 96 hours at the end of assay (FIG. 14A). No significant effect was observed of DL-2 on the nuclear expression of IMPα1 (see, FIG. 14A). Indicating this was specific for interaction of PDL1-PTM1 and IMPα1.

Given that these data show that DL-2 successfully inhibits the co-localization of PDL1-PTM1 and IMPα1 the inventors then confirmed the inhibition of this complex with DUOLINK analysis which detects closely interacting proteins. Strikingly, DL-2 is able to abrogate the complex of PDL1 and IMPα1 in a dose dependent manner, significantly impacting this complex and subsequently nuclear PDL1-PTM1 (FIG. 14B).

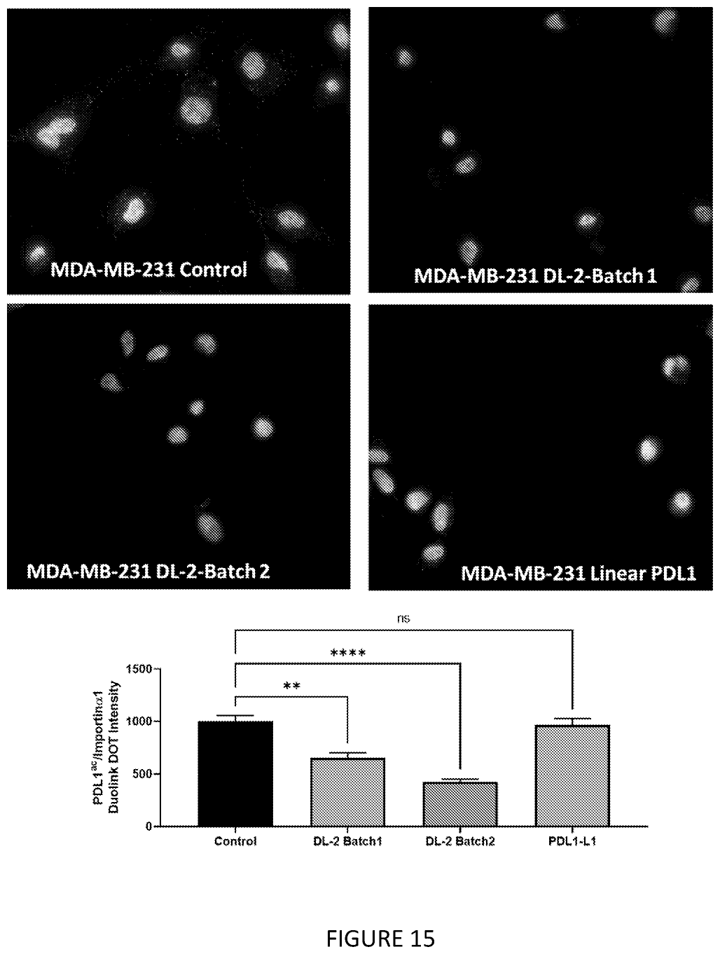

Accordingly, the inventors then set to establish the importance of the bicyclic peptide structure versus a native linear isoform. Strikingly, DL-2 was able to abrogate the complex of PDL1-PTM1 and IMPα1 in the human MDA-MB-231 cell line (metastatic, immunotherapy resistant). Incubation with DL-2 significantly impacts the complex and reducing the interaction of PDL1-PTM1 and IMPα1 (FIG. 15). Conversely, the linear peptide isoform (“PDL1-L1”, see Table 10 for sequence) was unable to significantly prevent the complex of PDL1-PTM1 and IMPα1.

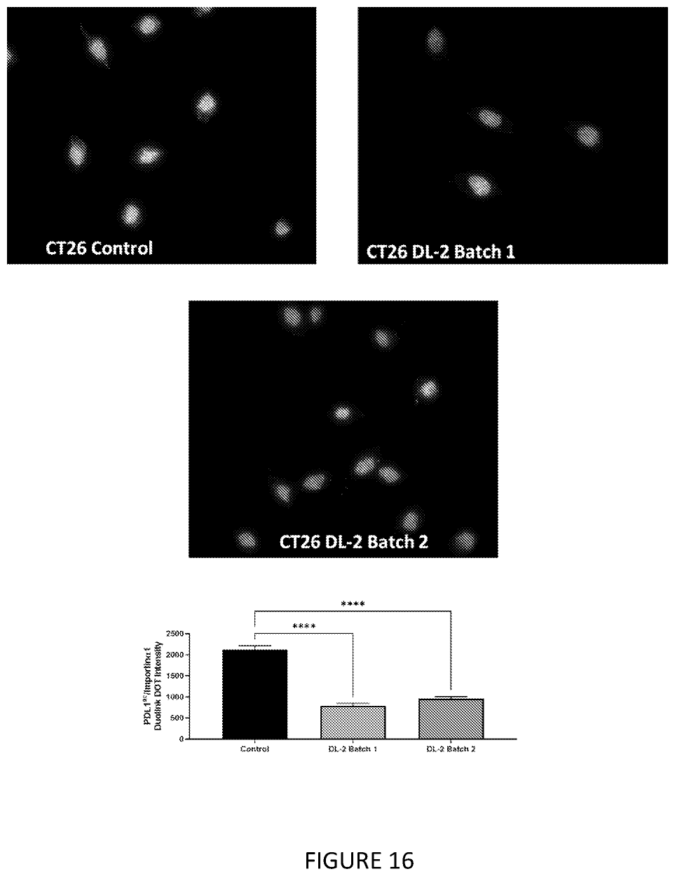

The inventor next assessed the ability of the DL-2 bicyclic peptide to interfere with the PDL1-PTM1 and IMPα1 complex in a human CT26 cell line (immunotherapy responsive cells) (FIG. 16). The DL-2 bicyclic peptide was able to successfully abrogate the complex of PDL1-PTM1 and IMPα1 in the CT26 immunotherapy responsive cell line, significantly impacting this complex and reducing the interaction of PDL1-PTM1 and IMPα1.

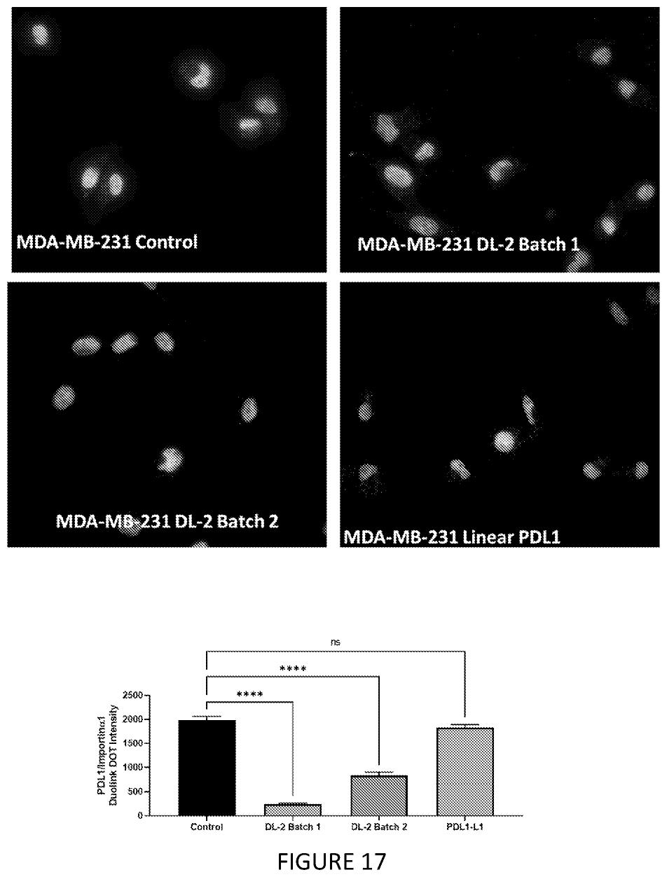

Given that these data strongly evidence that DL-2 successfully inhibits the PDL1-PTM1/IMPα1 complex the present inventor next investigated the ability of DL-2 to inhibit the interaction between the unmodified PDL1 and IMPα1. DL-2 was able to abrogate the complex of PDL1 (unmodified) and IMPα1, in MDA-MB-231 cells, significantly impacting this complex and reducing the interaction of PDL1-Ac and IMPα1 (FIG. 17). Again, the linear PDL1 peptide isoform inhibitor was unable to significantly inhibit this complex.

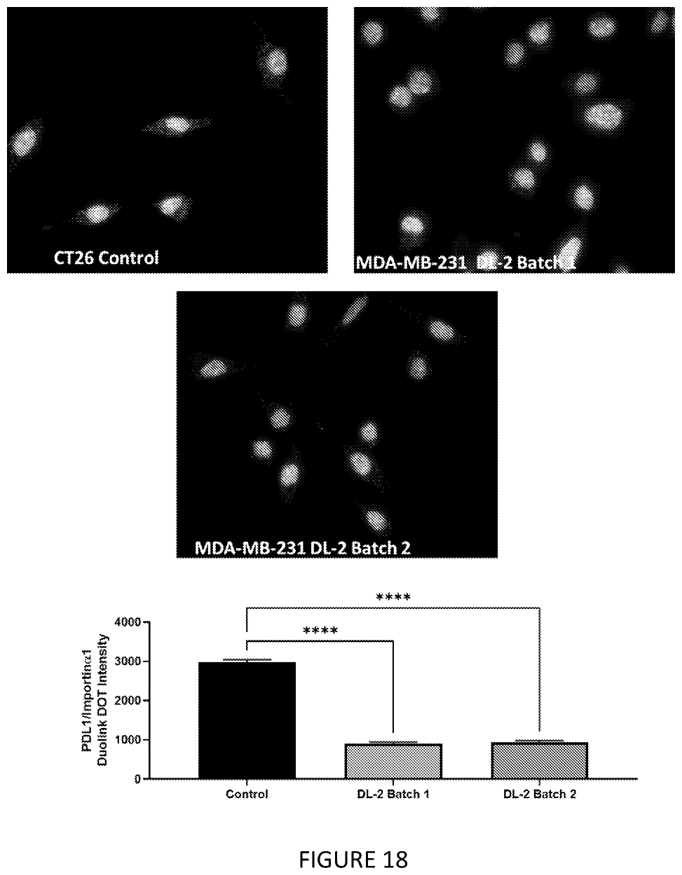

The present inventors next assessed the ability of bicyclic peptide DL-2 to interfere with the PDL1 (unmodified) and IMPa1 complex in a human CT26 cell line (immunotherapy responsive cells), similarly to per FIG. 17. DL-2 was able to abrogate the complex of PDL1 (unmodified) and IMPα1, in CT26 cells significantly impacting this complex and reducing the interaction of PDL1 and IMPα1 (FIG. 18).

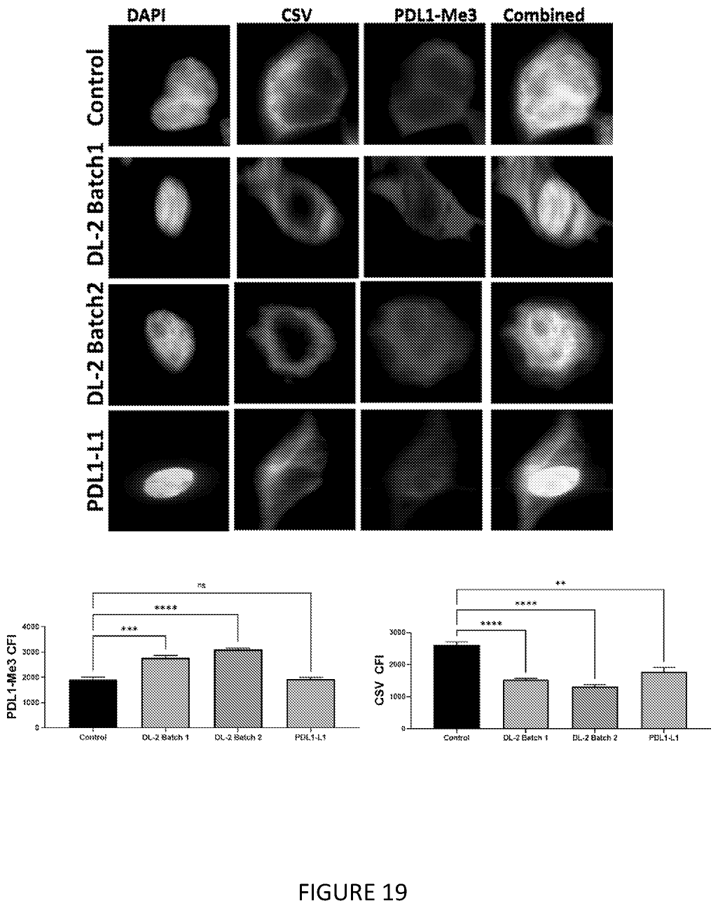

These results demonstrate that DL-2 is able to successfully inhibit the nuclear translocation of PDL1 by blocking the interaction between PDL1 and the importin pathway. Based on these data, the inventor investigated the effect of the blocking the nuclear axis of PDL1 on the cytoplasmic-biased PTM of PDL1, PDL1-PTM2. Enrichment of PDL1-PTM2 in the cytoplasmic fraction resembles cancer responsive to immunotherapy and our structural analysis above supports that this form has reduced efficiency in the importin pathway and with its ligand PD1 (FIG. 19). DL-2 was able to both inhibit the mesenchymal marker CSV, and induce increased cytoplasmic expression of PDL1-PTM2 (PDL1-Me3). The linear PDL1 peptide was able to reduce expression of CSV with far less significance and was not able to induce any increased expression of PDL1-PTM2

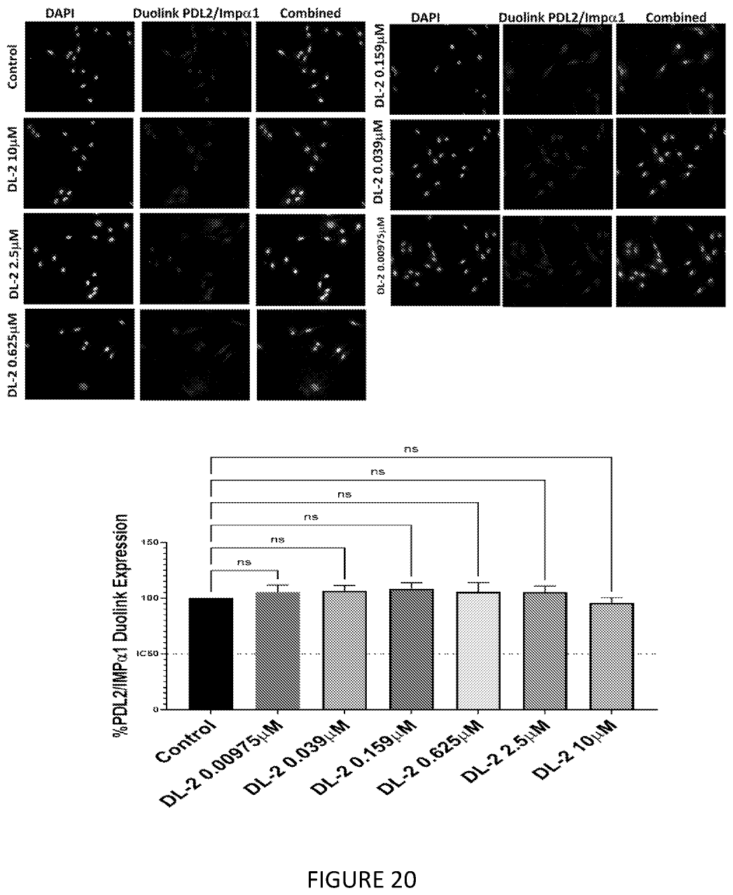

The present inventor then looked to assess the specificity of DL-2 for targeting PDL1. As such, it was investigated whether DL-2 is able to interfere with the interaction of PDL2 and the importin pathway. Strikingly, DL-2 had no significant effect on the PDL2:IMPα1 complex at any concentration, demonstrating the specificity of DL-2 for its target PDL1/PDL1-PTM1 and interaction with IMPα1 (FIG. 20).

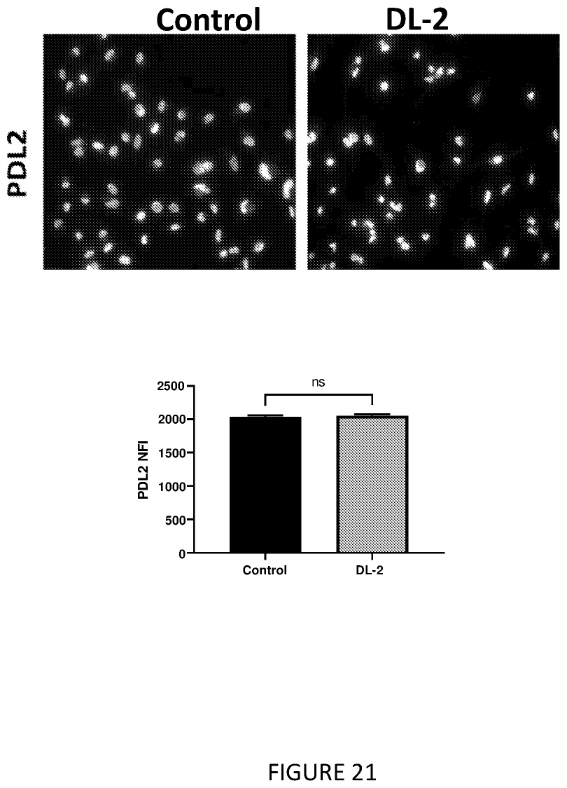

Based on the data from FIG. 20, the present inventor sought to further confirm the specificity of DL-2 by examining the effect of DL-2 on the expression of nuclear PDL2. DL-2 had no effect at all on the nuclear expression of PDL2 (FIG. 21).

To further investigate the specificity of DL-2 the present inventors investigated the effect of DL-2 on a series of nuclear associated proteins. No off-target effects were observed by DL-2 on any of the analysed nuclear proteins (FIG. 21). This provides clear evidence demonstrating that DL-2 is specific for PDL1/PDL1-PTM1 and its interaction with IMPα1 only, with no off-target effects demonstrated.

### Materials & Methods

### Determining PD-1/PD-L1 Pathway

All data reduction and integration were performed using iMosflm93 and merging and scaling in Aimless94. Molecular replacement in PhaserMR95, search model90 was used to build a structure, and the final structure was generated using iterative cycles of manual building in Coot96 and refinement in Phenix97,98. The Camilla bipartite Matrix was used for density. The proteins used in this modelling are listed below.

TABLE 8

| PDB Acc. No. | Protein Structure |
|---|---|
| 3BIK | PD-1/PD-L1 complex. |
| 5IUS | Human PD-L1 in complex with high |
| | affinity PD-1 mutant. |
| 4Z18 | Human PD-L1. |
| 3FN3 | Dimeric structure of PD-L1. |
| 5J89 | Human PD-L1 with low molecular mass inhibitor. |

### Importin-α Pathway Screening Assay

MDA-MB-231 cells were treated with DL-2 for 4 to 96 hours or control peptide scramble and were permeabilized probed a mouse anti-IMPα1, rabbit anti-PDL1 and visualized with a donkey anti-rabbit secondary antibody conjugated to Alexa Fluor 647 or a donkey anti-mouse secondary antibody conjugated to Alexa Fluor 568. Cover slips were mounted on glass microscope slides with Prolong nucleus glass Antifade reagent (Life Technologies). PDL1 and IMPα1 digital images were analyzed using ImageJ software (ImageJ, NIH, Bethesda, MD, USA) to determine the mean fluorescent intensity (mean FI). Graphs represent the mean FI of IMPα1. Graphs also depict the PCC of PDL1/IMPα1 using ImageJ software with automatic thresholding and manual selection of regions of interest (ROIs) specific for cell nuclei to calculate the Pearson's co-efficient correlation (PCC) for each pair of antibodies. PCC values range from: −1=inverse of co-localization.

### DUOLINK Proximity Assay

MDA-MB-231 cells were treated with DL-2; with concentrations ranging from 10 μM to 0.00975 μM. MDA-MB-231 cells were treated with DL-2 or Control and were permeabilized and were probed with the DUOLINK ligation assay. Cover slips were mounted on glass microscope slides with Prolong nucleus glass Antifade reagent (Life Technologies). PDL1-Ac and IMPα1 DUOLINK Digital images were analyzed using ImageJ software (ImageJ, NIH, Bethesda, MD, USA) and graphs represent the mean DOT Fluorescent Intensity in the nucleus or the cytoplasm compartments with significant differences calculated as per Kruskal-Wallis one-way ANOVA. The IC50 is marked with a red dotted line and corresponds to 0.039 μM of DL-2.

To characterize unmodified PDL1 and IMPα1, MDA-MB-231 cells or CT26 cells were treated with vehicle control, DL-2-batch 1, DL-2-batch 2 or linear PDL1. Cells were permeabilised and were probed with the DUOLINK ligation assay. Cover slips were mounted on glass microscope slides with ProLong nucblue glass Antifade reagent (Life Technologies). PDL1 (unmodified) and IMPα1 DUOLINK Digital images were analysed using ImageJ software (ImageJ, NIH, Bethesda, MD, USA) and graphs represent the mean DOT Fluorescent Intensity in the nucleus or the cytoplasm compartments with significant differences calculated as per One-Way ANOVA comparison.

### PDL1-PTM1—IMPα1 Binding Assay

MDA-MB-231 of were treated with vehicle control, DL-2-Batch 1, DL-2-Batch 2, or linear PDL1 peptide. Cells were permeabilised and were probed with the DUOLINK ligation assay. Cover slips were mounted on glass microscope slides with Prolong nucleus glass Antifade reagent (Life Technologies). PDL1Ac and IMPα1 DUOLINK Digital images were analysed using ImageJ software (ImageJ, NIH, Bethesda, MD, USA) and graphs represent the mean DOT Fluorescent Intensity in the nucleus or the cytoplasm compartments with significant differences calculated as per One-Way ANOVA comparison.

### High Resolution Digital Pathology—CSV Expression

MDA-MB-231 were permeabilized and treated with either vehicle control or, DL-2 (current stock), or linear PDL1 (PDL1-L1). The change in expression of CSV or PDL1-Me3 was characterized with high resolution digital pathology. Significant differences are calculated as one-way ANOVA Kruskal-Wallis test comparison to vehicle control. CFI=cytoplasmic fluorescent intensity.

### Binding Specificity Assay

MDA-MB-231 cells were treated with DL-2; with concentrations ranging from 10 μM to 0.00975 μM. Cover slips were mounted on glass microscope slides with Prolong nucleus glass Antifade reagent (Life Technologies). PDL2 and IMPα1 DUOLINK Digital images were analyzed using ImageJ software (ImageJ, NIH, Bethesda, MD, USA) and graphs represent the mean DOT Fluorescent Intensity in the nucleus or the cytoplasm compartments with significant differences calculated as per Kruskal-Wallis one-way ANOVA.

### Immunofluorescence Microscopy

Demonstrating via fluorescent intensity in MDA-MB-231 brain cancer cell line of no effect at all on expression of PDL2. The cells were fixed and immunofluorescence microscopy was performed. Digital images were analysed using ImageJ software (ImageJ, NIH, Bethesda, MD, USA) to determine the mean nuclear fluorescent intensity (TNFI) in cells measured using ImageJ to select the nucleus minus background (n=>500 cells/sample).

### Determination of Nuclear-Associated Proteins

MDA-MB-231 cells were treated with DL-2 with concentration of 10 μM. MDA-MB-231 cells were treated with DL-2 or control and were permeabilised before being probed with the antibodies specific for PKCθ, LSD1, p65, C-Rel, SET, pSTAT3. Cover slips were mounted on glass microscope slides with ProLong nucblue glass Antifade reagent (Life Technologies). Digital images were analysed using ImageJ software (ImageJ, NIH, Bethesda, MD, USA) and graphs represent the mean fluorescence intensity in the nucleus or the cytoplasm compartments with significant differences calculated as per Mann-Whitney pairwise comparison.

### Example 6

### DL-2 Maintains Potency Using Alternative Bicyclic Scaffolds

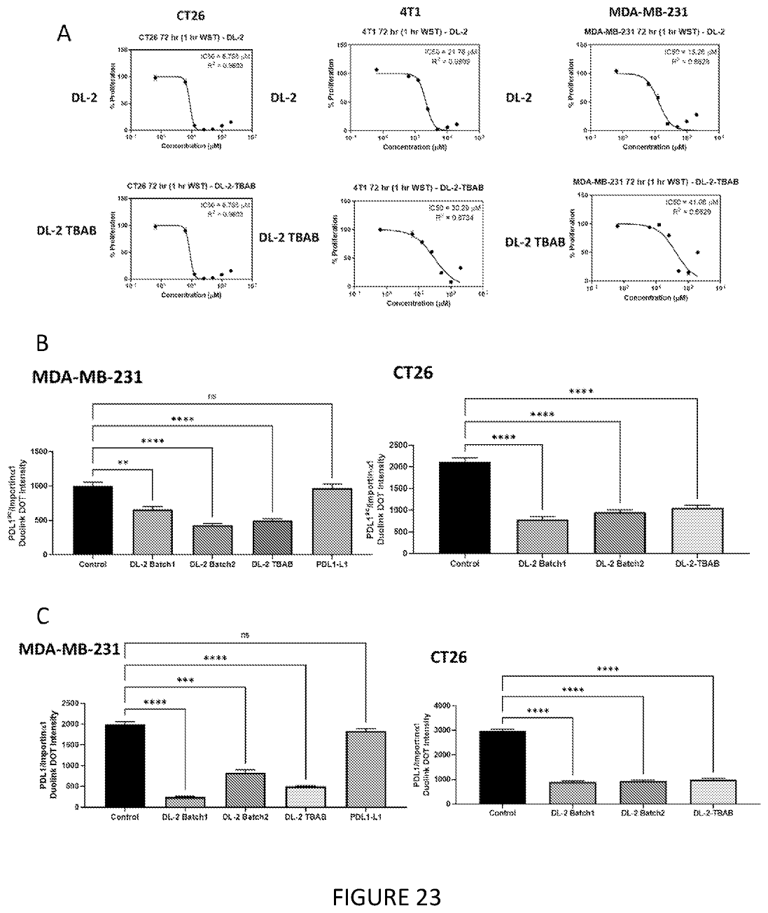

Given the importance of the bicyclic structure for the DL-2 peptide to effect its target, the present inventor sought to investigate any effect that an alternative molecular scaffold would have on its activity. Accordingly, an equivalent polypeptide sequence to DL-2 was generated on a TBAB molecular scaffold and its activity assessed. Strikingly, as observed in FIG. 23, both DL-2-TBMB and DL-2-TBAB were able to effectively inhibit the proliferation of human metastatic cancer cell lines responsive or resistant to immunotherapy (FIG. 23A). Furthermore, both molecular scaffolds were able to inhibit the complex of PDL1-PTM1 and IMPα1 (FIG. 23B) as well as the complex of PDL1-unmodified and IMPα1 (FIG. 23C), in both MDA-MB-231 and CT26 cell lines. This shows a significant impacting on this complex, providing further evidence of a substantial reduction of the interaction of PDL1-unmodified and IMPα1. This is in stark contrast with a linear PDL1 (PDL1-L1) peptide inhibitor, which was unable to significantly impact the complex of PDL1-unmodified and IMPα1 (FIGS. 23A, and B).

### Materials & Methods

(A) CT26, 4T1 or MDA-MB-231 Cells were treated with inhibitors for 72 hours. Following inhibition, media was removed and replaced with WST-1 cell proliferation reagent. Absorbance was recorded at 450 nm after 1 hour. Data represent a single independent experiment performed in triplicate. Results are graphed as mean +/− standard error (SE). B) MDA-MB-231 or CT26 cancer cells were treated with vehicle control, DL-2-Batch 1 (original stock), DL-2-Batch 2 (current stock), DL-2-TBAB or linear PDL1. Cells were permeabilised and were probed with the DUOLINK ligation assay. Cover slips were mounted on glass microscope slides with ProLong nucblue glass Antifade reagent (Life Technologies). PDL1Ac and IMPα1 DUOLINK Digital images were analysed using ImageJ software (ImageJ, NIH, Bethesda, MD, USA) and graphs represent the mean DOT Fluorescent Intensity in the nucleus or the cytoplasm compartments with significant differences calculated as per one-way ANOVA comparison. (C) MDA-MB-231 or CT26 cancer cells were treated with vehicle control, DL-2-Batch 1 (Original stock), DL-2-Batch 2 (current stock), DL-2-TBAB or linear PDL1. Cells were permeabilised and were probed with the DUOLINK ligation assay. Cover slips were mounted on glass microscope slides with ProLong nucblue glass Antifade reagent (Life Technologies). PDL1-unmodified and IMPα1 DUOLINK digital images were analysed using ImageJ software (ImageJ, NIH, Bethesda, MD, USA) and graphs represent the mean DOT fluorescent intensity in the nucleus or the cytoplasm compartments with significant differences calculated as per one-way ANOVA comparison.

### Example 7

### Functional Characterisation of D-Amino Acid Isoforms of DL-2

DL-2-D represents the D-isomer version of the DL-2 peptide inhibitor, wherein all the amino acids of the bicyclic peptide DL-2 and composed of the D-isomer form (except glycine which has no D-isomer form) (as shown in Table 9). In brief, reflecting the L-amino acids by a mirror are their enantiomers, D-amino acids, which share identical chemical and physical properties of L-amino acids, except for their ability to rotate plane-polarized light in opposite directions. While L-amino acids serve as the elements of the natural proteins translated in the ribosome, D-amino acids are found in some post-translational modification and peptidoglycan cell walls of bacteria. Unlike L-amino acids, D-amino acids rarely act as the substrates of endogenous proteases, resulting in the resistance of D-peptides towards proteolysis.

In order to determine whether the stability of DL-2 could be increased (whilst retaining binding affinity and efficacy) a D-isomer version of DL-2 was designed, termed “DL-2-D” (Table 9).

TABLE 9

| Position (1) for | Linear | Bicycle Peptide | Bicycle |
|---|---|---|---|
| Linear Peptide | Sequence | Sequence | DL-2-D |
| Z₁ | | A | a |
| | | Cysteine | c |
| X₁ | L | L | l |
| X₂ | T | T | t |
| X₃ | F | F | f |
| X₄ | I | I | i |
| X₅ | F | F | f |
| | | Cysteine | c |
| X₆ | R | R | r |
| X₇ | L | L | l |
| X₈ | R | R | r |
| X₉ | K | K | k |
| X₁₀ | G | G | G |
| X₁₁ | R | R | r |
| | | Cysteine | c |
| X₁₂ | M | M | m |
| X₁₃ | M | M | m |
| X₁₄ | D | D | d |
| X₁₅ | V | V | v |
| X₁₆ | K | K | k |
| Z₂ | K | K | k |

Bolded residues indicate amino acids that are different to the native linear PDL1 NLS sequence. Lower case letters represent D-amino acid isoforms.

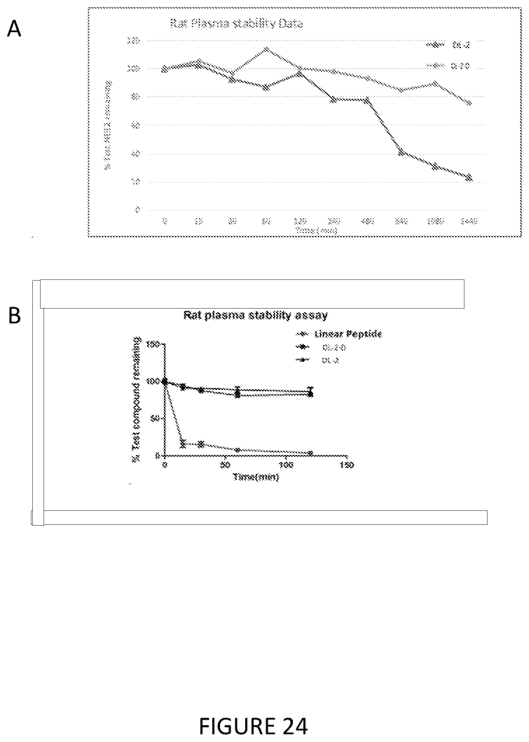

To investigate the stability of DL-2-D as compared to DL-2, the plasma stability both the bicyclic peptides was determined in rat plasma (FIG. 24). These stability data clearly demonstrate that the DL-2-D bicyclic peptide is significantly more stable than the DL-2 bicyclic peptide (FIG. 24A). As expected, both bicyclic peptides were more stable than the native linear NLS sequence of PDL1 (FIG. 24B).

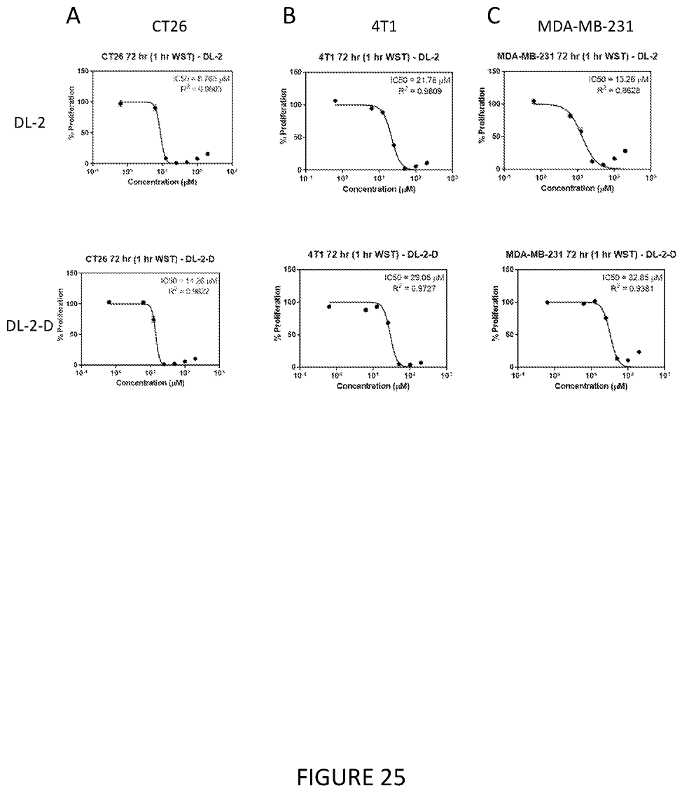

Based on this confirmed stability data, showing an enhanced stability of D-amino acid formulations, the ability of DL-2-D to inhibit cancer cell proliferation was then analysed (FIG. 25). Both DL-2 and DL-2-D bicyclic peptides significantly inhibit the proliferation of the three different cancer cell lines.

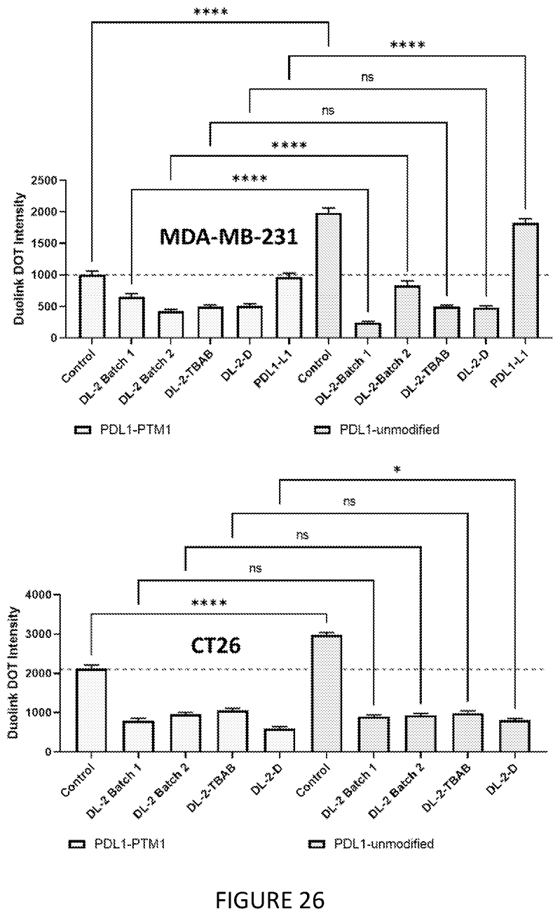

The present inventor next looked to determine whether DL-2-D retained the ability to target the complex of PDL1-PTM1 and IMPα1, and the complex of PDL1 (unmodified) and IMPα1 using the DUOLINK assay which detects closely interacting proteins as already established for DL-2 (FIG. 26). Both complexes are significantly inhibited the complex formation by treatment with both forms (i.e., D-isomer and L-isomer) of bicyclic peptides, whereas there is no change with the linear prototype PDL1-L1.

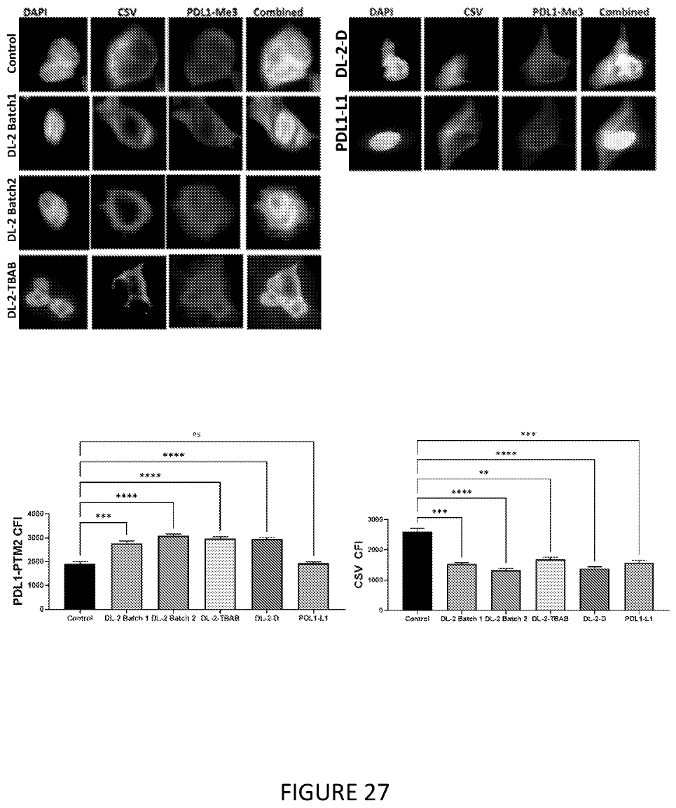

Next, to confirm that DL-2-D and the DL-2 scaffolds enrich PDL1-PTM2 (as demonstrated above for DL-2) the effect of the blocking the nuclear axis of PDL1 on the cytoplasmic-biased PTM of PDL1, PDL1-PTM2, was investigated. Enrichment of PDL1-PTM2 in the cytoplasmic fraction is indicative of a cancer that responsive to immunotherapy and structural rationale above supports that this form has reduced efficiency in the importin pathway, and with its ligand PD1. Both of the bicyclic peptide inhibitors were able to inhibit the mesenchymal marker CSV, and induce increase expression of cytoplasmic expression of PDL1-PTM2 (FIG. 27). In stark contrast, the linear PDL1 peptide was able to reduce expression of CSV, but did not induce any increased expression of PDL1-PTM2.

### Summary

A brief summary of the efficacy of each of the candidate peptides described above is provided in Tables 10-12, below.

TABLE 10
DL-2 Linear Peptide Derivatives

| | | DL-2 Linear (Peptide | | Potency |
|---|---|---|---|---|
| | | Derivative) | Modification | (CSC inhibition) |
| PDL1-L1 | Myristyl- | LTFIFRLRKGRMMDVKK | Prototype | Weak |
| DL2-Linear | Myristyl- | ACLTFIFCRLRKGRCMMDVKK | DL2 Linear | No change in |
| | | | | potency relative |
| | | | | to prototype |
| DL2-D- | Myristyl- | acltfifcrlrkGrcmmdvkk | DL2-D Linear | No change in |
| Linear | | | | potency relative |
| | | | | to prototype |
| DL2-D Ret | Myristyl- | kkvdmmcrGkrlrcfiftlca | Whole sequence | No change in |
| Linear | | | replaced with D- | potency relative |
| | | | isomers in | to prototype |
| | | | retroinverso | |
| | | | format | |
| DL2-D-Sar | Myristyl- | acltfifcrlrk(138)rcmmdvkk | Whole sequence | No change in |
| Linear | | | replaced with D- | potency relative |
| | | | isomers with | to prototype |
| | | | Sarcosine | |
| | | | (methylated | |
| | | | Glycine (138) | |

TABLE 11
DL-2 Linear Cyclic Peptide Derivatives

| | | | Linker | |
|---|---|---|---|---|
| | | | amino | |
| | | Sequence | acids | Comments |
| PDL1-L1 | Myristyl- | LTFIFRLRKGRMMDVKKAG | 2 | Synthesis failed. |
| C1 | | | | |
| PDL1-L1 | Myristyl- | LTFIFRLRKGRMMDVKKAGGA | 4 | Failed to inhibit |
| C2 | | | | mesenchymal cancer |
| | | | | markers and nuclear |
| | | | | PDL1-PTM1 in human |
| | | | | cancer lines. |
| PDL1-L1- | Myristyl- | LTFIFRLRKGRMMDVKKAGGAAG | 6 | Synthesis failed. |
| C3 | | | | |
| PDL1-L1- | Myristyl- | GAAGAGLTFIFRLRKGRMMDVKKAG | 8 | Synthesis failed. |
| C4 | | | | |

Underlined/bolded residues represent linker residues.

TABLE 12
DL-2 Bicyclic Peptide Derivatives

| | | Sequence | Backbone | Comments |
|---|---|---|---|---|
| DL-1 | Myristyl- | LTFICFRLRKGRMCMDVCGKK | TBMB | DL-1 was 3-fold less |
| | | | | potent than DL-2 at |
| | | | | inhibiting cancer cell |
| | | | | proliferation. |
| DL-2 | Myristyl- | ACLTFIFCRLRKGRCMMDVKK | TBMB | Potent inhibition of |
| | | | | cancer cell proliferation |
| | | | | (~100-fold greater than |
| | | | | linear prototype). |
| DL-3 | Myristyl- | ACLTFIFRLRCKGRMCMDVGKK | TBMB | DL-3 displayed no |
| | | | | inhibition of cancer cell |
| | | | | proliferation (due to |
| | | | | disruption of critical |
| | | | | NLS region residues). |
| DL-2-D | Myristyl- | acltfifcrirckGrcmmdvkk | TBMB | Potent inhibition of |
| | | | | cancer cell proliferation. |

Underlined/bolded residues represent linker residues.

### Materials & Methods

### Rat Plasma Stability Assay

The stability assay was performed as per CRO's protocol (Creative Peptides). In brief, peptide stability assays were carried out using a rat plasma model with the following steps:
3.6 mL of human plasma and rat plasma were thawed at room temperature and centrifuged at 3000 rpm for 15 min. Remove the precipitate.
Prepare 20 mL termination solution (20 mL acetonitrile+20 mL formic acid) and precool at 4° C.
Prepare the following solution:

| Components | Human | Rat |
|---|---|---|
| Plasma (from step 1) | Human plasma | Rat plasma |
| | 3440 mL | 3440 mL |
| 3.5 mM (10 mg/ml) Bicyclic | 60 μL | 60 μL |
| Peptide | (Final conc. | (Final conc.: |
| | 60 μM) | 60 μM) |
| Total volume | 3.5 mL | 5.5 mL |

Incubate the tube at 37° C. Take out 200 μL plasma sample at desired time points up to 24 hours respectively (e.g., at 0, 15, 30, 60, 120 mins, etc). Add 400 μL pre-cooled stop solution, centrifuge at 3000 rpm, 4° C. for 15 min, and carefully aspirate the supernatant.
The supernatant samples were filtered with a 0.22 μm filter membrane, and then analysed by LC-MS in order to determine the percentage of the test compound remaining.

### Cell Proliferation Assay

4T1, CT26 or MDA-MB-231 cancer cell lines were treated with DL-2 or DL-2-D bicyclic inhibitors for 72 hours. Following inhibition, media was removed and replaced with WST-1 cell proliferation reagent. Absorbance was recorded at 450 nm after 1 hour. Data represent a single independent experiment performed in triplicate; results are graphed as mean +/− standard error (SE).

### PDL1-Importin Binding Assay

MDA-MB-231 and CT26 cells were treated with vehicle control, DL-2-Batch 1, DL-2-Batch 2, DL-2-TBAB (TBAB scaffold), DL-2-D (D-Isomer) or linear PDL1. This figure depicts the comparison via high resolution imaging using 100× objective with the ASI digital pathology system of DUOLINK cells stained with PDL1 (unmodified) and IMPα1, and PDL1-PTM1 and IMPα1. Cells were permeabilised and were probed with the DUOLINK ligation assay. Cover slips were mounted on glass microscope slides with ProLong nucblue glass Antifade reagent (Life Technologies). PDL1 (unmodified) and IMPα1 and PDL1-PTM1 and IMPα1 DUOLINK digital images were analysed using ImageJ software (ImageJ, NIH, Bethesda, MD, USA) and graphs represent the mean DOT Fluorescent Intensity in the nucleus or the cytoplasm compartments with significant differences calculated as per One-Way ANOVA comparison. The red cut-off represents the highest level of expression for the PDL1-PTM1 and IMPα1 complex.

### Detection of Cytoplasmic PDL1-PTM2 and CSV

The MDA-MB-231 breast cancer cell line were treated with either vehicle control or with DL-2-Batch 1 (original stock), DL-2-Batch 2 (current stock), DL-2-TBAB (TBAB scaffold), DL-2-D (D-Isomer), or linear PDL1. Two batches were analyses on order to ensure the reproducibility of results. Samples were permeabilized and labelled with antibodies for PDL1-PTM2 and CSV. Samples were visualized using the ASI Digital Pathology system. The change in expression of CSV or PLD1-Me3 was characterized with high resolution digital pathology. Significant differences are calculated as one-way ANOVA Kruskal-Wallis test comparison to vehicle control. CFI=cytoplasmic fluorescent intensity.

### Example 8

### “Head-to-Tail” Cyclic Peptide Inhibitors Do Not Inhibit Mesenchymal Cancer Markers

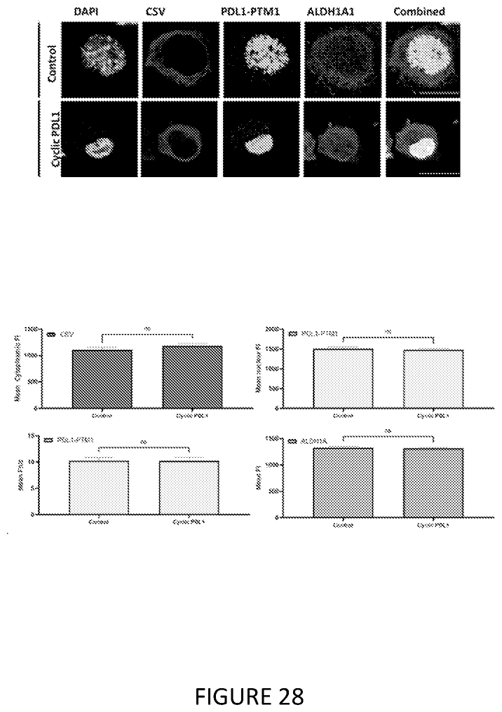

In order to establish the importance of the bicyclic structure of DL-2, as opposed to any alternative methodologies, a traditional “head-to-tail” cyclized PDL1-P1 peptide was generated. The cyclic PDL1 peptide was unable to impact on the level of nuclear expression of PDL1-PTM1, or impact on the expression of CSV or ALDH1A (FIG. 28).

These data confirm the importance of having a bicyclic structure for targeting the nuclear pathway of PDL1.

### Materials & Methods

“Head-to-tail” cyclisation of the peptide, which joins the C-terminal amino acid to the N-terminal amino acid (i.e., which is the classic approach for cyclisation) was considered and carried out by the manufacturer's protocol. Synthesis of this cyclized peptide was highly problematic and produced a low yield. The maximum yield achieved was only 1-2 mg.

Breast cancer cell line, 4T1, was treated with vehicle control or cyclic PDL1 peptide (cyclic PDL1) at a concentration of 50 μM. Cells were permeabilised and were probed with primary antibodies against PDL1-PTM1, CSV and ALDH1A1. Cover slips were mounted on glass microscope slides with ProLong nucblue glass Antifade reagent (Life Technologies). PDL1-PTM1, CSV and ALDH1A1 digital images were analysed using ImageJ software (ImageJ, NIH, Bethesda, MD, USA). Graphs represent the mean fluorescent intensity in the nucleus or the cytoplasm compartments with significant differences calculated as per Mann-Whitney analysis or the ratio of nuclear to cytoplasmic staining for PDL1-PTM1 which was calculated using the formula F(n−b)/(c−b) wherein n=nuclear staining, b=background, c=cytoplasmic. A ratio of 1 or high indicates nuclear bias, under 1 indicates cytoplasmic bias.

### Example 10

### In Vivo Studies of DL-2

DL-2 monotherapy (20 and 30 mg/kg) reduces primary tumour volume in the 4T1 model of metastatic breast cancer over an eight day treatment period. Tumour weight is also significantly reduced at 20 and 30 mg/kg after 8 days. No change in body weight is observed with DL-2 treatment. monotherapy does not alter lung, liver or spleen weights.

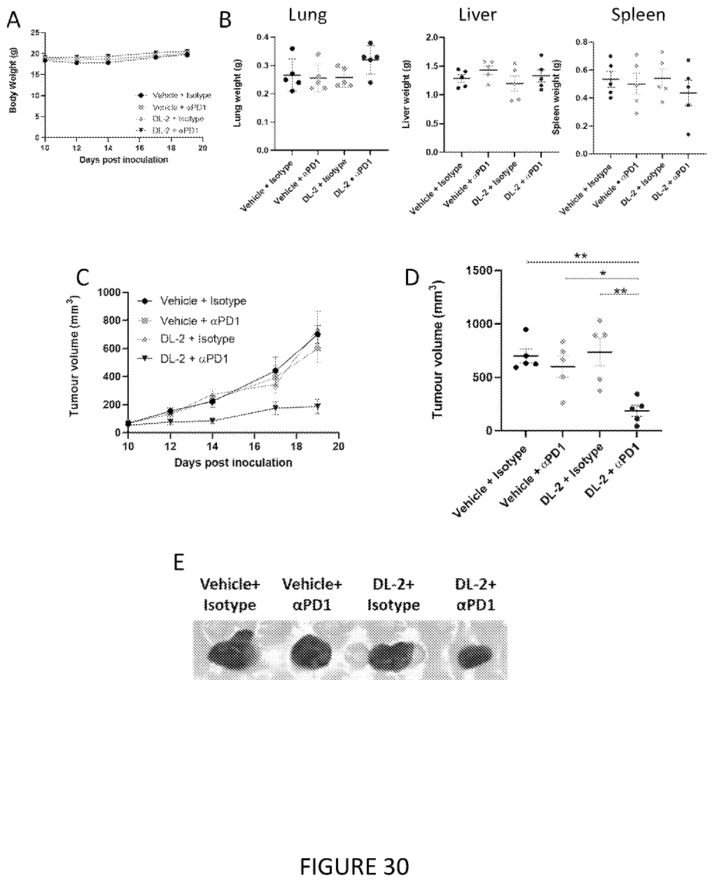

DL-2 treatment in combination with an anti-PD1 antibody significantly reduces primary tumour volume in the 4T1 model over a 10 day treatment period. No change in body weight is observed with DL-2 and anti-PD1 combination therapy. DL-2 in combination with anti-PD1 does not alter lung, liver or spleen weights (FIG. 30).

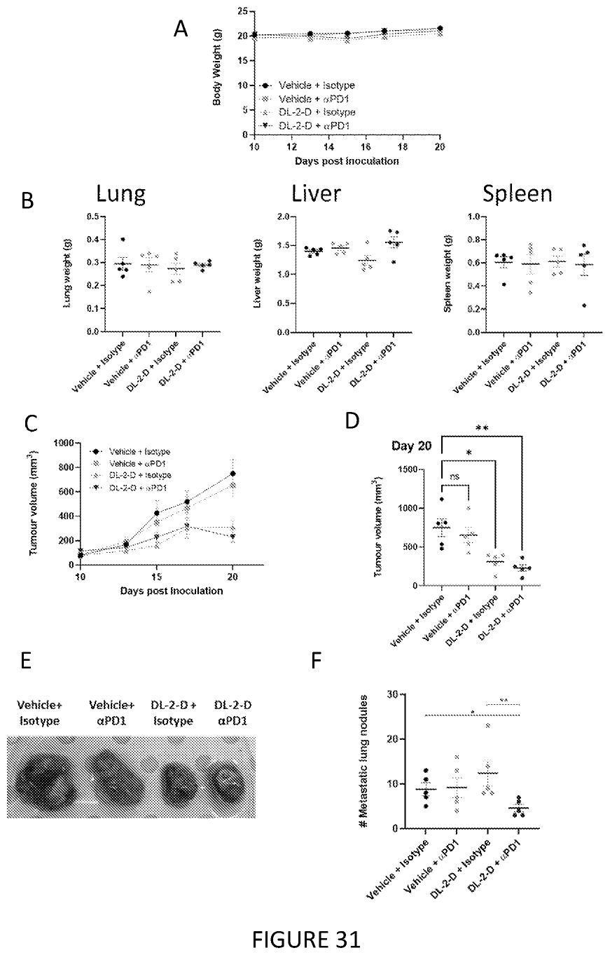

Based on this data, the inventor analysed whether DL-2-D treatment as a monotherapy or in combination with anti-PD1 significantly reduces primary tumour volume in the 4T1 model over a 10 day treatment period as well as lung metastasis. No change in body weight is observed with DL-2-D and anti-PD1 combination therapy or DL-2-D monotherapy. DL-2-D alone or in combination with anti-PD1 does not alter lung, liver or spleen weights (FIG. 31).

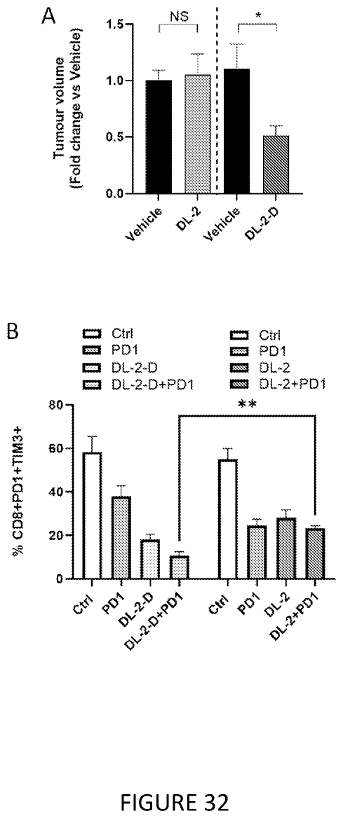

To assess the side by side efficacy of DL-2 vs DL-2-D the inventor compared DL-2 and DL-2-D at an equivalent dosing regimen as described in above. These data, presented in FIG. 32 clearly shows that DL-2-D significantly inhibits tumour volume as a monotherapy agent. This finding provides evidence that DL-2-D has improved stability and elimination half-life, which could therefore lead to even more effective treatments. The effect of DL-2-D or DL-2 on immunotherapy-resistant CD8+ TIM3+ T cells from a 4T1 TNBC tumour mouse model was examined via FACS. Surprisingly, DL-2-D was more potent than DL-2 at abrogating the TIM3+ immunotherapy-resistant signature in CD8+ T cells, re-programming them for a functional, effector signature.

### Materials & Methods

### In Vivo Mouse Models

4T1 Gold standard immunotherapy resistant mouse model mice obtained from the ARC, Perth and mice were inoculated with 4T1 TNBC metastatic cancer cells. DL-2 is supplied lyophilised in glass vials and was reconstituted using sodium chloride injection BP (0.9% saline) to make a stock solution of 10 mg/mL using a needle/syringe, DL-2 was mixed by inversion. Animals were then injected by intraperitoneal injection at 10, 20 or 30 mg/kg daily and rested over the weekend. Mice were monitored over time for body weight and tumour volume.

### PD1 Immunotherapy Combination Treatments

4T1 Gold standard immunotherapy resistant mouse model mice obtained from the ARC, Perth and mice were inoculated with 4T1 TNBC metastatic cancer cells. DL-2 or DL-2-D is supplied lyophilised in glass vials was reconstituted using sodium chloride injection BP (0.9% saline) to make a stock solution of 10 mg/mL using a needle/syringe, DL-2 was mixed by inversion. Animals were then injected by intraperitoneal injection and mice were monitored over time for body weight and tumour volume for DL-2 at 20 mg/kg every second day with resting over the weekend in conjunction with αPD1 or isotype control (10 mg/kg).

DL-2-D was mixed by inversion. Animals were then injected by intraperitoneal injection and mice were monitored over time for body weight and tumour volume for DL-2-D at a concentration of 20 mg/kg every two days (identical regime to αPD1) in conjunction with αPD1 or isotype control (10 mg/kg).

### Example 12

### Effect of DL-2 on Mesenchymal Signature of MICs

Based on the efficacy of DL-2 inhibiting a mesenchymal signature, the present inventor sought to probe the effectiveness of DL-2 in inhibiting the immunotherapy resistant mesenchymal signature in metastatic initiating cells (MICs) isolated from stage IV metastatic cancer patients. MICs are found in liquid biopsies from patients with aggressive cancers and these cancer cells (MICs) can express a mesenchymal phenotype including the expression of resistance and metastatic markers such as ABCB5 and ALDHA1A1 as well as the novel PDL1-PTM1. MICs are also key mediators of metastatic events and resistant to immunotherapy.

The present inventor demonstrated in the above Examples that DL-2 inhibits an immunotherapy-resistant metastatic signature. These findings led the inventor to hypothesise that DL-2 would potentially inhibit these signatures in MICs from patient liquid biopsies. Upon investigation, it was found that DL-2 inhibited expression of immunotherapy-resistant, mesenchymal markers ABCB5 and ADLHA1A1, as well as PDL1-PTM1, in cancer cells from NSCLC, TNBC and melanoma patients. Accordingly, DL-2 will also inhibit seeding of metastasis by targeting and inhibiting these MICs.

### Materials & Methods

Mesenchymal MICs were isolated from TNBC, lung cancer and melanoma patient liquid biopsies (scored for RECIST v1.1 for immunotherapy/therapeutic resistance or responsiveness (Complete Response, Partial Response or Progressive Disease) and pre-clinically screened with either vehicle control or the nuclear PDL1 inhibitor, DL-2. Samples were fixed and immunofluorescence microscopy performed on these cells with primary antibodies targeting CSV, PDL1-PTM1 (acetylated nuclear PDL1), ALDH1A1 and ABCB5. Graph represents the CFI values for CSV, NFI for PDL1- PTM1, ALDH1A1 and FI for ABCB5 measured using the ASI Digital pathology automated system to select the nucleus minus background (n≥40 cells/sample/5 patients a group).

### Example 11

### Nanoparticle Encapsulation Methods

To optimize and enhance the stability and efficacy of DL-2 bicyclic peptide the present inventors utilized the Nanoprecision Systems nanoparticle delivery system. For this approach we have packaged DL-2 peptide inhibitor into a lipid-based nanoparticle. Briefly, a lipid mix was created, the peptide was dissolved in PBS pH 7.4 at a 10 mg/ml concentration then run them through various peptide to lipid ratios and flow rates in a nanoprecipitation method using the Precision Nanosystems NanoAssemblr Ignite. Following this we characterized their size distribution by intensity on a Malvern Panalytical Zetasizer Ultra DLS system.

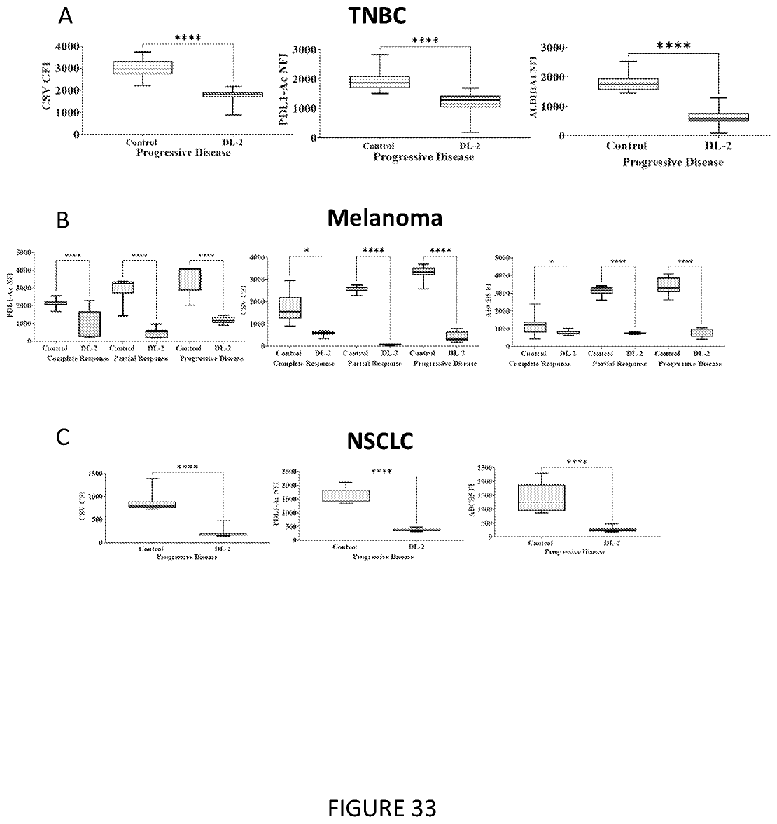

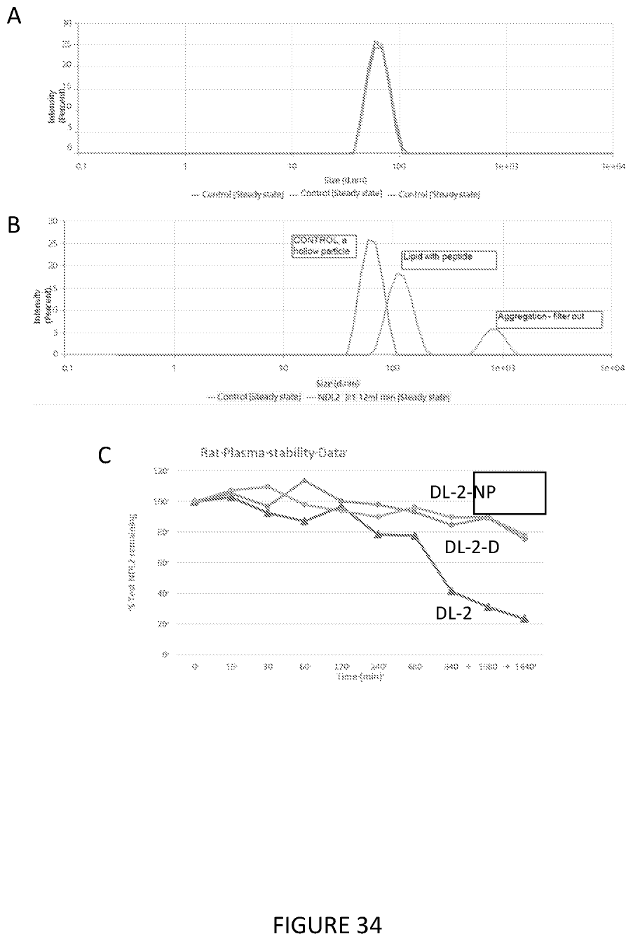

During the purification of the nanoparticle and DL-2 bicyclic peptide, it was clear that the vast majority of DL-2 had been successfully encapsulated (FIG. 33A,B). With reference to FIG. 34A-B demonstrates that in the plotted data that the peak at around 100 nm is the lipid nanoparticle with the contribution of the peptide whereas the control (hollow particle) is smaller in size.

Following purification, stability studies were performed in order to understand whether the encapsulated bicyclic peptide formulations would have higher stability in vivo than the un-encapsulated DL-2 bicyclic peptide. DL-2 nanoparticle (DL-2-NP) and DL-2-D are both significantly more stable than the naked DL-2 bicyclic peptide (FIG. 34C).

### Materials & Methods

### Preparation of Lipid Nanoparticles

Nanoparticles were created using standard methodologies in the art. To briefly summarise the process, a lipid mix was created using POPC (1-palmitoyl-2-oleoyl-sn-glycero-3-phosphocholine), cholesterol and DSPE-PEG (1,2-distearoyl-sn-glycero-3-phosphoethanolamine-N-[amino(polyethylene glycol)-2000]). DL-2 bicyclic inhibitor is solubilised in phosphate buffered saline (PBS) pH 7.4, at a 10 mg/ml concentration. The peptide was then run through at various flow rate ratios and flow rates against the lipids in a nanoprecipitation method using the Precision Nanosystems NanoAssemblr Ignite. Following this, the size was assessed on a Malvern Panalytical Zetasizer Ultra DLS system.

### Rat Stability Assay

The stability assay was performed using the same protocol as described above in Example 7.

### Example 12

### DL-2-NP Inhibits Cancer Cell Proliferation

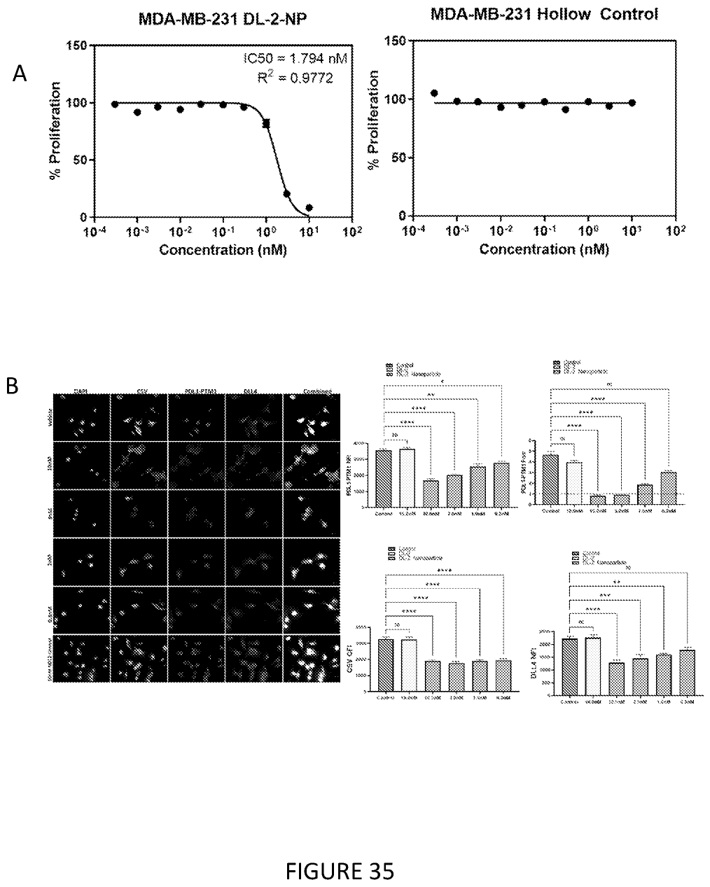

DL-2-NP was over 800-fold more potent the DL-2 when the IC50s of each compound were compared at inhibiting the proliferation of MDA-MB-231 (FIG. 35A).

DL-2-NP is able to abrogate the proliferation of the TNBC cancer cell line MDA-MB-231 in the nanomolar range, which is significantly lower concentration than DL-2 without lipid encapsulation. DL-2-NP is able to abrogate nuclear PDL1-PTM and DLL4 (implicated in maintenance and proliferation of cancerstem cells (CSC) and linked with poor patient survival) in a dose dependent manner (FIG. 35B). DL-2-NP also significantly abrogated CSV (cell surface vimentin, a mesenchymal marker of invasive cancer) at all concentrations tested (FIG. 35B). Treatment with DL-2-NP was also able to bias expression of PDL1-PTM1 to the cytoplasm in a dose dependent manner (red dotted line represents Fn/c of 1).

The inventor also found that DL-2-NP was more potent than the naked DL-2 at abrogating the PDL1 and IMPα1 complex, with a IC50 of only 1 nM for DL-2-NP (as compared to 39 nM for DL-2, indicating a ˜39-fold).

### Materials & Methods

### Cell Proliferation Assay

Cells weretreated with inhibitors for 48 hours. Following inhibition, media was removed and replaced with WST-1 cell proliferation reagent. Absorbance was recorded at 450 nm after 1 hour.

### PDL1-IMPα1 Binding Assay

MDA-MB-231 cells were treated with vehicle control (hollow nanoparticle), DL-2-NP at a concentration of 10 nM to 0.3 nM. Cells were permeabilised and were probed with the DUOLINK ligation assay. Cover slips were mounted on glass microscope slideswith ProLong nucblue glass Antifade reagent (Life Technologies). PDL1 (unmodified) & IMPα1 DUOLINK digital images were analysed using ImageJ software (ImageJ, NIH, Bethesda, MD, USA) and graphs represent the mean DOT Fluorescent Intensity in the nucleus or the cytoplasm compartments with significant differences calculated as per One-WayANOVA comparison.

The disclosure of every patent, patent application, and publication cited herein is hereby incorporated herein by reference in its entirety.

The citation of any reference herein should not be construed as an admission that such reference is available as “Prior Art” to the instant application.

Throughout the specification the aim has been to describe the preferred embodiments of the invention without limiting the invention to any one embodiment or specific collection of features. Those of skill in the art will therefore appreciate that, in light of the instant disclosure, various modifications and changes can be made in the particular embodiments exemplified without departing from the scope of the present invention. All such modifications and changes are intended to be included within the scope of the appended claims.

What is claimed is:

1. A PD-L1 bicyclic peptide mimetic comprising a polypeptide that comprises at least three cysteine residues, separated by at least two loop sequences, and a molecular scaffold which forms covalent bonds with the cysteine residues of the polypeptide such that at least two polypeptide loops are formed on the molecular scaffold, wherein the PD-L1 bicyclic peptide mimetic comprises an amino acid sequence of:
X₁C₁LX₂X₃IFC₂X₄LRKGX₅C₃X₆X₇X₈X₉KX₁₀
or a modified derivative, or pharmaceutically acceptable salt, thereof;
wherein:
C₁, C₂, and C₃ represent first, second and third cysteine residues, respectively;
X₁ is absent or alanine;
X₂ is selected from any small amino acid (optionally, threonine, glycine, serine, or alanine)
X₃ is selected from any amino acid;
X₄ is selected from any amino acid;
X₅ is selected from any amino acid;
X₆ is selected from any nonpolar/neutral amino acid (e.g., methionine, alanine, leucine, proline, glycine, isoleucine, phenylalanine, tryptophan, valine, and norleucine);
X₇ is selected from any nonpolar/neutral amino acid (e.g., methionine, alanine, proline, leucine, glycine, isoleucine, phenylalanine, tryptophan, valine, and norleucine);
X₈ is selected from any amino acid;
X₉ is selected from any nonpolar/neutral amino acid (e.g., valine, alanine, glycine, methionine, leucine, proline, isoleucine, phenylalanine, tryptophan, and norleucine); and
X₁₀ is selected from any amino acid.

2. The PD-L1 bicyclic peptide mimetic of claim 1, wherein X₂ is selected from threonine and alanine.

3. The PD-L1 bicyclic peptide mimetic of claim 1 or claim 2, wherein X₂ is threonine.

4. The PD-L1 bicyclic peptide mimetic of any one of claims 1-3, wherein X₃ is selected from phenylalanine and alanine.

5. The PD-L1 bicyclic peptide mimetic of any one of claims 1-4, wherein X₃ is phenylalanine.

6. The PD-L1 bicyclic peptide mimetic of any one of claims 1-5, wherein X₄ is selected from arginine and alanine.

7. The PD-L1 bicyclic peptide mimetic of any one of claims 1-6, wherein X₄ is arginine.

8. The PD-L1 bicyclic peptide mimetic of any one of claims 1-7, wherein X₅ is selected from arginine and alanine.

9. The PD-L1 bicyclic peptide mimetic of any one of claims 1-8, wherein X₅ is arginine.

10. The PD-L1 bicyclic peptide mimetic of any one of claims 1-9, wherein X₆ is selected from methionine, alanine, leucine and proline.

11. The PD-L1 bicyclic peptide mimetic of any one of claims 1-10, wherein X₆ is methionine.

12. The PD-L1 bicyclic peptide mimetic of any one of claims 1-11, wherein X₇ is selected from methionine, alanine, and proline.

13. The PD-L1 bicyclic peptide mimetic of any one of claims 1-12, wherein X₇ is methionine.

14. The PD-L1 bicyclic peptide mimetic of any one of claims 1-13, wherein X₇ is alanine.

15. The PD-L1 bicyclic peptide mimetic of any one of claims 1-14, wherein X₈ is selected from aspartic acid, alanine, glycine and valine.

16. The PD-L1 bicyclic peptide mimetic of any one of claims 1-15, wherein X₈ is aspartic acid.

17. The PD-L1 bicyclic peptide mimetic of any one of claims 1-16, wherein X₉ is selected from valine, alanine, glycine, methionine.

18. The PD-L1 bicyclic peptide mimetic of any one of claims 1-17, wherein X₉ is valine.

19. The PD-L1 bicyclic peptide mimetic of any one of claims 1-18, wherein X₁₀ is selected from lysine, alanine, asparagine, and methionine.

20. The PD-L1 bicyclic peptide mimetic of any one of claims 1-19, wherein X₁₀ is lysine.

21. The PD-L1 bicyclic peptide mimetic of any one of claims 1-20, wherein the peptide binds to importin.

22. The PD-L1 bicyclic peptide mimetic of any one of claims 1-21, wherein the peptide mimetic prevents or disrupts the complex between PD-L1 and importin.

23. The PD-L1 bicyclic peptide mimetic of any one of claims 1-22, comprising the amino acid sequence: ACLTFIFCRLRKGRCMMDVKK [SEQ ID NO: 1].

24. The PD-L1 bicyclic peptide mimetic of any one of claims 1-23, wherein the molecular scaffold is 1,3,5-(tribroMoMethyl)benzene) or TBAB.

25. The PD-L1 bicyclic peptide mimetic of any one of claims 1-24, further comprising an N-terminal cell-penetrating peptide.

26. The PD-L1 bicyclic peptide mimetic of claim 25, wherein the cell-penetrating peptide is Myr.

27. The PD-L1 bicyclic peptide mimetic of any one of claims 1-26, wherein the modified derivative includes one or more modifications selected from: N-terminal and/or C-terminal modifications; replacement of one or more amino acid residues with one or more non-natural amino acid residues (such a replacement of one or more polar amino acids with one or more isosteric or isoelectronic amino acids; replacement or one or more hydrophobic amino acid residues with other non-natural isosteric or isoelectronic amino acids); addition of a spacer group; replacement of one or more oxidation resistant amino acid residues; replacement of one or more amino acid residues with an alanine, replacement of one or more L amino acid residues with one or more D-amino acid residues; N-alkylation of one or more aminide bonds within the bicyclic peptide ligand; replacement of one or more peptide bonds with a surrogate bond; peptide backbone length modification; substitution of the hydrogen on the α-carbon of one or more amino acid residues with another chemical group, and post-synthetic biorthogonal modification of amino acids such as cysteine, lysine, glutamate and tyrosine with suitable amine, thiol, carboxylic acid and phenol-reactive reagents.

28. The PD-L1 bicyclic peptide mimetic of any one of claims 1-27, wherein the modified derivative comprises an N-terminal modification, such as an N-terminal acetyl group.

29. The PD-L1 bicyclic peptide mimetic of any one of claims 1-28, wherein the modified derivative comprises a C-terminal modification, such as a C-terminal amide group.

30. The PD-L1 bicyclic peptide mimetic of any one of claims 1-29, wherein the modified derivative comprises replacement of one or more amino acid residues with one or more non-natural amino acid residues.

31. The PD-L1 bicyclic peptide mimetic of any one of claims 1-30, wherein the modified derivative comprises one or more D-amino acid.

32. The PD-L1 bicyclic peptide mimetic of any one of claims 1-31, wherein substantially of the amino acids are D-amino acids.

33. The PD-L1 bicyclic peptide mimetic of any one of claims 1 to 32, wherein the peptide is formulated in a lipid nanoparticle.

34. The PD-L1 bicyclic peptide mimetic of any one of claims 1-28, wherein the pharmaceutically acceptable salt is selected from a hydrochloride or acetate salt.

35. A pharmaceutical composition comprising the PD-L1 bicyclic peptide mimetic of any one of claims 1-29, in combination with one or more excipients.

36. A method of reducing nuclear localization of PD-L1 in a PD-L1 overexpressing cell, the method comprising contacting the cell with a PD-L1 bicyclic peptide mimetic with the amino acid sequence of:
X₁C₁LX₂X₃IFC₂X₄LRKGX₅C₃X₆X₇X₈X₉KX₁₀
or a modified derivative, or pharmaceutically acceptable salt, thereof;
wherein:
C₁, C₂, and C₃ represent first, second and third cysteine residues, respectively;
X₁ is absent or alanine;
X₂ is selected from any small amino acid (optionally, threonine, glycine, serine, or alanine)
X₃ is selected from any amino acid;
X₄ is selected from any amino acid;
X₅ is selected from any amino acid;
X₆ is selected from any nonpolar/neutral amino acid (e.g., methionine, alanine, leucine, proline, glycine, isoleucine, phenylalanine, tryptophan, valine, and norleucine);
X₂ is selected from any nonpolar/neutral amino acid (e.g., methionine, alanine, proline, leucine, glycine, isoleucine, phenylalanine, tryptophan, valine, and norleucine);
X₈ is selected from any amino acid;
X₉ is selected from any nonpolar/neutral amino acid (e.g., valine, alanine, glycine, methionine, leucine, proline, isoleucine, phenylalanine, tryptophan, and norleucine); and
X₁₀ is selected from any amino acid.

37. The method of claim 36, wherein X₂ is selected from threonine and alanine.

38. The method of claim 36 or claim 37, wherein X₂ is threonine.

39. The method of any one of claims 36-38, wherein X₃ is selected from phenylalanine and alanine.

40. The method of any one of claims 36-39, wherein X₃ is phenylalanine.

41. The method of any one of claims 36-40, wherein X₄ is selected from arginine and alanine.

42. The method of any one of claims 36-41, wherein X₄ is arginine.

43. The method of any one of claims 36-42, wherein X₅ is selected from arginine and alanine.

44. The method of any one of claims 36-43, wherein X₅ is arginine.

45. The method of any one of claims 36-44, wherein X₆ is selected from methionine, alanine, leucine and proline.

46. The method of any one of claims 36-45, wherein X₆ is methionine.

47. The method of any one of claims 36-46, wherein X₇ is selected from methionine, alanine, and proline.

48. The method of any one of claims 36-47, wherein X₇ is methionine.

49. The method of any one of claims 36-48, wherein X₇ is alanine.

50. The method of any one of claims 36-49, wherein X₈ is selected from aspartic acid, alanine, glycine and valine.

51. The method of any one of claims 36-50, wherein X₈ is aspartic acid.

52. The method of any one of claims 36-51, wherein X₉ is selected from valine, alanine, glycine, methionine.

53. The method of any one of claims 36-52, wherein X₉ is valine.

54. The method of any one of claims 36-53, wherein X₁₀ is selected from lysine, alanine, asparagine, and methionine.

55. The method of any one of claims 36-54, wherein X₁₀ is lysine.

56. The method of any one of claims 36-55, comprising the amino acid sequence: ACLTFIFCRLRKGRCMMDVKK [SEQ ID NO: 1].

57. The method of any one of claims 36-56, wherein the molecular scaffold is 1,3,5-(tribroMoMethyl)benzene) or TBAB.

58. The method of any one of claims 36-57, wherein the modified derivative comprises one or more D-amino acid.

59. The method of any one of claims 36-58, wherein substantially of the amino acids are D-amino acids.

60. The method of any one of claims 36 to 59, wherein the peptide is formulated in a lipid nanoparticle.

61. The method of any one of claims 36-60, further comprising an N-terminal cell-penetrating peptide.

62. The method of claim 61, wherein the cell-penetrating peptide is myristic acid.

63. A method of treating or preventing cancer in a subject, wherein the cancer comprises at least one PD-L1 overexpressing cell, comprising administering to the subject a PD-L1 bicyclic peptide mimetic of any one of claims 1-35.

64. The method of any one of claims 36-63, wherein the PD-L1 overexpressing cell is a cancer cell, cancer stem cell, or a non-cancer stem cell tumour cell.

65. The method of claim 64, wherein the PD-L1 overexpressing cell is a cancer stem cell tumour cell.

66. The method of any one of claims 63-65, wherein the cancer is selected from breast, prostate, lung, bladder, pancreatic, colon, liver, or brain cancer, or melanoma, or retinoblastoma.

67. The method of any one of claims 36-66, further comprising administering at least one further cancer therapies.

68. The method of claim 67, wherein the further cancer therapy is a chemotherapeutic agent.

69. A composition comprising a PD-L1 bicyclic peptide mimetic with the amino acid sequence of:
X₁C₁LX₂X₃IFC₂X₄LRKGX₅C₃X₆X₇X₈X₉KX₁₀
or a modified derivative, or pharmaceutically acceptable salt, thereof;
wherein:
C₁, C₂, and C₃ represent first, second and third cysteine residues, respectively;
X₁ is absent or alanine;
X₂ is selected from any small amino acid (optionally, threonine, glycine, serine, or alanine)
X₃ is selected from any amino acid;
X₄ is selected from any amino acid;
X₅ is selected from any amino acid;
X₆ is selected from any nonpolar/neutral amino acid (e.g., methionine, alanine, leucine, proline, glycine, isoleucine, phenylalanine, tryptophan, valine, and norleucine);
X₇ is selected from any nonpolar/neutral amino acid (e.g., methionine, alanine, proline, leucine, glycine, isoleucine, phenylalanine, tryptophan, valine, and norleucine);
X₈ is selected from any amino acid;
X₉ is selected from any nonpolar/neutral amino acid (e.g., valine, alanine, glycine, methionine, leucine, proline, isoleucine, phenylalanine, tryptophan, and norleucine); and
X₁₀ is selected from any amino acid;
for use in therapy.

70. A composition comprising a PD-L1 bicyclic peptide mimetic with the amino acid sequence of:
X₁C₁LX₂X₃IFC₂X₄LRKGX₅C₃X₆X₇X₈X₉KX₁₀
or a modified derivative, or pharmaceutically acceptable salt, thereof;
wherein:
C₁, C₂, and C₃ represent first, second and third cysteine residues, respectively;
X₁ is absent or alanine;
X₂ is selected from any small amino acid (optionally, threonine, glycine, serine, or alanine)
X₃ is selected from any amino acid;
X₄ is selected from any amino acid;
X₅ is selected from any amino acid;
X₆ is selected from any nonpolar/neutral amino acid (e.g., methionine, alanine, leucine, proline, glycine, isoleucine, phenylalanine, tryptophan, valine, and norleucine);
X₇ is selected from any nonpolar/neutral amino acid (e.g., methionine, alanine, proline, leucine, glycine, isoleucine, phenylalanine, tryptophan, valine, and norleucine);
X₈ is selected from any amino acid;
X₉ is selected from any nonpolar/neutral amino acid (e.g., valine, alanine, glycine, methionine, leucine, proline, isoleucine, phenylalanine, tryptophan, and norleucine); and
X₁₀ is selected from any amino acid;
for use in the treatment of cancer.

71. Use of a composition comprising a PD-L1 bicyclic peptide mimetic with the amino acid sequence:
X₁C₁LX₂X₃IFC₂X₄LRKGX₅C₃X₆X₇X₈X₉KX₁₀
or a modified derivative, or pharmaceutically acceptable salt, thereof;
wherein:
C₁, C₂, and C₃ represent first, second and third cysteine residues, respectively;
X₁ is absent or alanine;
X₂ is selected from any small amino acid (optionally, threonine, glycine, serine, or alanine)
X₃ is selected from any amino acid;
X₄ is selected from any amino acid;
X₅ is selected from any amino acid;
X₆ is selected from any nonpolar/neutral amino acid (e.g., methionine, alanine, leucine, proline, glycine, isoleucine, phenylalanine, tryptophan, valine, and norleucine);
X₇ is selected from any nonpolar/neutral amino acid (e.g., methionine, alanine, proline, leucine, glycine, isoleucine, phenylalanine, tryptophan, valine, and norleucine);
X₈ is selected from any amino acid;
X₉ is selected from any nonpolar/neutral amino acid (e.g., valine, alanine, glycine, methionine, leucine, proline, isoleucine, phenylalanine, tryptophan, and norleucine); and
X₁₀ is selected from any amino acid;
in the manufacture of a medicament for the treatment of cancer.


SEQUENCE LISTING (111 sequences)

SEQ ID NO: 1 - protein, 21 aa, Homo sapien
ACLTFIFCRLRKGRCMMDVKK

SEQ ID NO: 2-21 - skipped (20 entries)

SEQ ID NO: 22 - protein, 11 aa, Homo Sapien
YGRKKRPQRRR

SEQ ID NO: 23 - protein, 16 aa, Homo Sapien
RRWRRWWRRWWRRWRR

SEQ ID NO: 24 - protein, 21 aa, Homo Sapien
CWKKKKKKKKKKKKKKKKKKK

SEQ ID NO: 25 - protein, 40 aa, Homo sapien
KKKKKKKKKKKKKKKKKKWCCWKKKKKKKKKKKKKKKKKK

SEQ ID NO: 26 - protein, 26 aa, Homo sapien
WTLNSAGYLLGKINLKALAALAKKIL

SEQ ID NO: 27 - protein, 13 aa, Homo sapien
GLFEALEELWEAK

SEQ ID NO: 28 - protein, 46 aa, Homo sapien
KKKKKKKKKKKKKKKKGGCRGDMFGCAKKKKKKKKKKKKKKKKRGD

SEQ ID NO: 29 - protein, 25 aa, Homo sapien
KKKKKKKKKKKKKKKKGGCMFGCGG

SEQ ID NO: 30 - protein, 31 aa, Homo sapien
KKKKKKKKKKKKKKKKICRRARGDNPDDRCT

SEQ ID NO: 31 - protein, 22 aa, Homo sapien
KKWKMRRNQFWVKVQRBAKBBA

SEQ ID NO: 32 - protein, 30 aa, Homo sapien
VAYISRGGVSTYYSDTVKGRFTRQKYNKRA

SEQ ID NO: 33 - protein, 32 aa, Homo sapien
IGRIDPANGKTKYAPKFQDKATRSNYYGNSPS

SEQ ID NO: 34 - protein, 21 aa, Homo sapien
KETWWETWWTEWSQPKKKRKV

SEQ ID NO: 35 - protein, 12 aa, Homo sapien
PLAEIDGIELTY

SEQ ID NO: 36 - protein, 30 aa, Homo sapien
KKKKKKKKKKKKKKKKGGPLAEIDGIELGA

SEQ ID NO: 37 - protein, 30 aa, Homo sapien
KKKKKKKKKKKKKKKKGGPLAEIDGIELCA

SEQ ID NO: 38 - protein, 28 aa, Homo sapien
GALFLGFLGGAAGSTMGAWSQPKSKRKV

SEQ ID NO: 39 - protein, 20 aa, Homo sapien
WEAKLAKALAKHLAKALKAC

SEQ ID NO: 40 - protein, 30 aa, Homo sapien
LARLLARLLARLLARLLARLLARLNHCHHH

SEQ ID NO: 41 - protein, 16 aa, Homo sapien
KLLKLLLKLWLLKLLL

SEQ ID NO: 42 - protein, 11 aa, Homo sapien
KKKKKKKKGGC

SEQ ID NO: 43 - protein, 11 aa, Homo sapien
KWKKKWKKGCC

SEQ ID NO: 44 - protein, 8 aa, Homo sapien
RWRRZGGC

SEQ ID NO: 45 - protein, 7 aa, Homo sapien
PKKKRKV

SEQ ID NO: 46 - protein, 12 aa, Homo sapien
PEVKKKRKPEYP

SEQ ID NO: 47 - protein, 12 aa, Homo sapien
TPPKKKRKVEDP

SEQ ID NO: 48 - protein, 13 aa, Homo sapien
GGGGPKKKRKVGG

SEQ ID NO: 49 - protein, 13 aa, Homo sapien
GGGFSTSLRARKA

SEQ ID NO: 50 - protein, 17 aa, Homo sapien
CKKKKKKSEDEYPYVPN

SEQ ID NO: 51 - protein, 28 aa, Homo sapien
CKKKKKKKSEDEYPYVPNFSTSLRARKA

SEQ ID NO: 52 - protein, 25 aa, Homo sapien
LVRKKRKTEEESPLKDKDAKKSKQE

SEQ ID NO: 53 - protein, 12 aa, Homo sapien
KGKZKGKGGGKG

SEQ ID NO: 54 - protein, 13 aa, Homo sapien
GRKKRRQRRRPPQ

SEQ ID NO: 55 - protein, 16 aa, Drosophila
RQIKIWFQNRRMKWKK

SEQ ID NO: 56 - protein, 21 aa, Homo sapien
TRSSRAGLQFPVGRVHRLLRK

SEQ ID NO: 57 - protein, 13 aa, Homo sapien
KRVSRNKSEKKRR

SEQ ID NO: 58 - protein, 18 aa, Homo sapien
KIALKIALKALKAALKIA

SEQ ID NO: 59 - protein, 16 aa, Homo sapien
AAVALLPAVLIALIAP

SEQ ID NO: 60 - protein, 6 aa, Homo sapien
VPMLKE

SEQ ID NO: 61 - protein, 5 aa, Homo sapien
VPMLK

SEQ ID NO: 62 - protein, 5 aa, Homo sapien
PMLKE

SEQ ID NO: 63 - protein, 4 aa, Homo sapien
PMLK

SEQ ID NO: 64 - protein, 28 aa, Mus musculus
MANLGYWLIALFVTMWTDVGLCKKRPKP

SEQ ID NO: 65 - protein, 18 aa, Homo sapien
LLIILRRRIRKQAHAHSK

SEQ ID NO: 66 - protein, 21 aa, Homo sapien
KETWWETWWTEWSQPKKKRKV

SEQ ID NO: 67 - protein, 18 aa, Homo sapien
RGGRLSYSRRRFSTSTGR

SEQ ID NO: 68 - protein, 27 aa, Homo sapien
GWTLNSAGYLLGKINLKAIAAIAKKIL

SEQ ID NO: 69 - protein, 21 aa, Homo sapien
AGYLLGKINLKALAALAKKIL

SEQ ID NO: 70 - protein, 20 aa, Homo sapien
GLWRALWRLLRSLWRLLWRA

SEQ ID NO: 71 - protein, 15 aa, Homo sapien
SDLWEMMMVSIACQY

SEQ ID NO: 72 - protein, 12 aa, Homo sapien
TSPLNIHNGQKL

SEQ ID NO: 73 - protein, 26 aa, Homo sapien
DPKGDPKGVTVTVTVTVTGKGDPKPD

SEQ ID NO: 74 - protein, 16 aa, Homo sapien
RVIRVWFQNKRCKDKK

SEQ ID NO: 75 - protein, 290 aa, Homo sapien
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 76-99 - skipped (24 entries)

SEQ ID NO: 100 - protein, 19 aa, Homo sapien
TFFYGGCRGKRNNFKTEEY

SEQ ID NO: 101 - protein, 19 aa, Homo sapien
TFFYGGSRGKRNNFKTEEY

SEQ ID NO: 102-103 - skipped (2 entries)

SEQ ID NO: 104 - protein, 19 aa, Homo sapien
TFFYGGSRGKRNNFKTEEY

SEQ ID NO: 105 - protein, 19 aa, Homo sapien
RFFYGGSRGKRNNFKTEEY

SEQ ID NO: 106 - protein, 19 aa, Homo sapien
RFFYGGSRGKRNNFKTEEY

SEQ ID NO: 107 - protein, 19 aa, Homo sapien
RFFYGGSRGKRNNFRTEEY

SEQ ID NO: 108 - protein, 19 aa, Homo sapien
TFFYGGSRGKRNNFRTEEY

SEQ ID NO: 109 - protein, 19 aa, Homo sapien
TFFYGGSRGRRNNFRTEEY

SEQ ID NO: 110 - protein, 20 aa, Homo sapien
CTFFYGGSRGKRNNFKTEEY

SEQ ID NO: 111 - protein, 20 aa, Homo sapien
TFFYGGSRGKRNNFKTEEYC